(19) United States
(12) Patent Application Publication
(10) Pub. No.: US 2025/0271435 A1
(43) Pub. Date: Aug. 28, 2025

(54) GLYCOPROTEIN ASSESSMENT

(71) Applicant: PrognomiQ, Inc., San Mateo, CA (US)

(72) Inventors: Bruce WILCOX, Keezletown, VA (US); Philip MA, San Jose, CA (US); Kavya SWAMINATHAN, Castro Valley, CA (US); Chi-Hung LIN, Redwood City, CA (US)

(21) Appl. No.: 18/815,648

(22) Filed: Aug. 26, 2024

Related U.S. Application Data
(63) Continuation of application No. PCT/US2023/063358, filed on Feb. 27, 2023.
(60) Provisional application No. 63/314,955, filed on Feb. 28, 2022.

(51) Int. Cl. G01N 33/574; G16H 10/40

(52) U.S. Cl. CPC G01N 33/57423; G01N 33/57488; G16H 10/40; G01N 2440/38; G01N 2458/15

(57) ABSTRACT

Described herein are methods for screening or testing for a disease state using a biological sample. The method may include using glycoprotein, glycopeptide or glycan measurements in evaluating a biological state. The measurements may be obtained through the use of nanoparticles that adsorb glycoproteins, glycopeptides, or glycans.

### CROSS-REFERENCE

This application is a continuation of PCT/US2023/063358 filed Feb. 27, 2023, which claims the benefit of U.S. Provisional Application Ser. No. 63/314,955 filed on Feb. 28, 2022, the entirety of which is hereby incorporated by reference herein.

### BACKGROUND

There is a need for methods of accurately detecting a disease or condition at an early stage of the disease or condition. Accurately detecting a disease or condition at an early stage can lead to effective treatments and improved prognosis for a subject having the disease or condition.

### SUMMARY

Disclosed herein, in some aspects, are methods that include the use of glycoprotein, glycopeptide, or glycan information. Disclosed herein, in some aspects, are methods, comprising: obtaining a data set comprising glycoprotein information from biomolecule coronas that correspond to physiochemically distinct particles incubated with a biofluid sample from a subject suspected of having a disease state; and applying a classifier to the data set to identify the biofluid sample as indicative of a healthy state or the disease state. Disclosed herein, in some aspects, are methods, comprising: (a) obtaining a data set comprising amounts of at least 10 glycoproteins or glycopeptides from biomolecule coronas that correspond to particles incubated with a biofluid sample from a subject; and (b) applying a classifier to the data set to identify the biofluid sample as indicative of cancer or as not indicative of cancer. Some aspects include identifying the subject as having the cancer. Some aspects include identifying administering a cancer treatment to the subject. In some embodiments, the cancer comprises lung cancer. In some embodiments, the lung cancer comprises non-small cell lung cancer (NSCLC). In some embodiments, the NSCLC comprises stage 1, stage 2, or stage 3 NSCLC. In some embodiments, the NSCLC comprises stage 4 NSCLC. In some embodiments, the data set comprises first measurements of a readout indicative of the presence, absence or amount of the at least 10 distinct glycoproteins or glycopeptides of the biomolecule coronas. In some embodiments, or glycopeptides further comprises generating second measurements having a sensitivity or specificity of about 80% or greater of being indicative of the subject having or not having the cancer. In some embodiments, obtaining the data set comprises detecting the at least 10 glycoproteins or glycopeptides by mass spectrometry, chromatography, liquid chromatography, high-performance liquid chromatography, solid-phase chromatography, a lateral flow assay, an immunoassay, an enzyme-linked immunosorbent assay, a western blot, a dot blot, immunostaining, sequencing or a combination thereof. In some embodiments, obtaining the data set comprises detecting the at least 10 glycoproteins or glycopeptides by the mass spectrometry. In some embodiments, the classifier comprises features to distinguish between early-stage NSCLC and late-stage NSCLC. In some embodiments, the classifier comprises a supervised data analysis, an unsupervised data analysis, a machine learning, a deep learning, a dimension reduction analysis, a clustering analysis, or a combination thereof. In some embodiments, the clustering analysis comprises a hierarchical cluster analysis, a principal component analysis, a partial least squares discriminant analysis, a random forest classification analysis, a support vector machine analysis, a k-nearest neighbors analysis, a naive bayes analysis, a K-means clustering analysis, a hidden Markov analysis, or a combination thereof. In some embodiments, the glycoproteins or glycopeptides comprise multiple glycosylated versions of a same protein or a same peptide, respectively. In some embodiments, the glycoproteins or glycopeptides comprise different proteins or different peptides, respectively.

Disclosed herein in some aspects, are methods, comprising: (a) contacting a sample (e.g. biofluid sample) of a subject with particles to form biomolecule coronas comprising at least 10 distinct glycoproteins or glycopeptides adsorbed to the particles; and (b) obtaining first measurements of the at least 10 distinct glycoproteins or glycopeptides. In some embodiments, obtaining the first measurements comprises combining the glycoproteins or glycopeptides with labeled or unlabeled glycoproteins or glycopeptides, or with labeled or unlabeled non-glycosylated forms of the glycoproteins or glycopeptides. In some embodiments, the method further comprises identifying second measurements as indicative of the subject having or not having have cancer. Some aspects include identifying the subject as having the cancer. Some aspects include identifying administering a cancer treatment to the subject. In some embodiments, the cancer comprises lung cancer. In some embodiments, the lung cancer comprises non-small cell lung cancer (NSCLC). In some embodiments, the NSCLC comprises stage 1, stage 2, or stage 3 NSCLC. In some embodiments, the NSCLC comprises stage 4 NSCLC. In some embodiments, the second measurements have a sensitivity or specificity of about 80% or greater of being indicative of the subject having or not having the cancer. In some embodiments, (b) comprises obtaining the first measurements of the at least 10 distinct glycoproteins or glycopeptides by mass spectrometry, chromatography, liquid chromatography, high-performance liquid chromatography, solid-phase chromatography, a lateral flow assay, an immunoassay, an enzyme-linked immunosorbent assay, a western blot, a dot blot, immunostaining, or sequencing, or a combination thereof. In some embodiments, (b) comprises obtaining the first measurements of the at least 10 distinct glycoproteins or glycopeptides by the mass spectrometry. In some embodiments, obtaining measurements of the at least 10 distinct glycoproteins comprise measuring a readout indicative of the presence, absence or amount of the at least 10 distinct glycoproteins of the biomolecule coronas.

Disclosed herein, in some aspects, are methods, comprising: (a) contacting a sample (e.g. biofluid sample) from a subject with particles to form a biomolecule corona comprising proteins or peptides and glycoproteins or glycopeptides adsorbed to the particles; and (b) enriching the glycoproteins or glycopeptides, or separating the glycoproteins or glycopeptides from the proteins or peptides. In some embodiments, separating the glycoproteins or glycopeptides from the proteins or peptides comprises using liquid chromatography to separate the glycoproteins or glycopeptides from the proteins or peptides. In some embodiments, the liquid chromatography comprises hydrophilic interaction liquid chromatography (HILIC), electrostatic repulsion liquid chromatography (ERLIC) enrichments, high performance liquid chromatography (HPLC), or a combination thereof. In some embodiments, the liquid chromatography comprises multidimensional liquid chromatography. In some embodiments, the multidimensional liquid chromatography comprises two-dimensional electrophoresis.

Disclosed herein, in some aspects, are methods, comprising: (a) contacting a sample (e.g. biofluid sample) from a subject with particles to form a biomolecule corona comprising glycoproteins adsorbed to the particles; and (b) combining the glycoproteins or glycopeptides with labeled glycoproteins or glycopeptides.

In some embodiments, (a) is performed prior to (b). In some embodiments, (a) is performed subsequent to (b). In some embodiments, (a) is performed during (b). In some embodiments, at least one of the glycoproteins or glycopeptides and at least one of the labeled glycoproteins or glycopeptides are the same. In some embodiments, at least one of the glycoproteins or glycopeptides and at least one of the labeled glycoproteins or glycopeptides are different. In some embodiments, the labeled glycoproteins or glycopeptides comprise an isotopic label, a mass tag, a barcode, a fluorescent label, a post-translation modification, a biomolecule from a same species of the subject, or a biomolecule from a species different than a species of the subject. In some embodiments, at least one of the labeled glycoproteins or glycopeptides have a predetermined amount. In some embodiments, each of the labeled glycoproteins or glycopeptides each have one predetermined amount. In some embodiments, the method further comprises measuring a readout indicative of the presence, absence or amount of: (1) the glycoproteins or glycopeptides, (2) the labeled glycoproteins or glycopeptides, (3) a combination thereof. In some embodiments, the method further comprises generating the readout indicative of the presence, absence or amount of the glycoproteins or glycopeptides by comparing thereof with the readout indicative of the presence, absence or amount of the labeled glycoproteins or glycopeptides. In some embodiments, the method further comprises normalizing the readout indicative of the presence, absence or amount of the glycoproteins or glycopeptides with the readout indicative of the presence, absence or amount of the labeled glycoproteins or glycopeptides. In some embodiments, the method further comprises generating a combined readout indicative of the presence, absence or amount of the glycoproteins or glycopeptides using the readouts indicative of the presence, absence or amount of the glycoproteins or glycopeptides and the labeled glycoproteins or glycopeptides. Some aspects include calculating a ratio of glycosylated glycoprotein or glycopeptide over a total amount of glycosylated and nonglycosylated glycoprotein or glycopeptide.

Disclosed herein, in some aspects, are methods that include contacting a sample from a subject with particles to form a biomolecule corona comprising glycoproteins or glycopeptides adsorbed to the particles; and releasing at least one glycan moiety from the glycoproteins or glycopeptides adsorbed to the particles. Some aspects include separating the at least one glycan moiety from the glycoproteins or glycopeptides. Some aspects include combining the at least one glycan moiety with a labeled glycan moiety. In some aspects, the at least one glycan moiety and the labeled glycan moiety are a same glycan moiety. In some aspects, the at least one glycan moiety and the labeled glycan moiety are different glycan moieties. Some aspects include measuring an amount of the at least one glycan moiety or the labeled glycan moiety. Some aspects include measuring an amount of the at least one glycan moiety or the labeled glycan moiety by mass spectroscopy. In some aspects, a step is conducted in the presence of heavy water comprising an isotope. In some aspects, the heavy water comprises deuterium or ¹⁸O. Some aspects include introducing the isotope to a glycosylation site of the glycoproteins or glycopeptides that is de-glycosylated subsequent to a release of the at least one glycan moiety from the glycoproteins or glycopeptides. Some aspects include measuring an amount of at least one de-glycosylated glycoprotein or glycopeptide labeled by the isotope and an amount of glycoproteins or glycopeptides that are not labeled. Some aspects include calculating a ratio of the amount of at least one de-glycosylated glycoprotein or glycopeptide labeled by the isotope and the amount of glycoproteins or glycopeptides that are not labeled. In some aspects, the ratio may comprise the amount of at least one de-glycosylated glycoprotein or glycopeptide labeled by the isotope divided by a total amount comprising the amount of at least one de-glycosylated glycoprotein or glycopeptide labeled by the isotope and the amount of glycoproteins or glycopeptides that are not labeled.

Disclosed herein, in some aspects, are methods that include particles. In some embodiments, the particles comprise at least 2 different particles. In some embodiments, the particles comprise at least 3, 4, 5 or more different particles. In some embodiments, the particles comprise physiochemically distinct particles. In some embodiments, the physiochemically distinct particles comprise lipid particles, metal particles, silica particles, or polymer particles. In some embodiments, the physiochemically distinct particles comprise carboxylate particles, poly acrylic acid particles, dextran particles, polystyrene particles, dimethylamine particles, amino particles, silica particles, or N-(3-trimethoxysilylpropyl)diethylenetriamine particles.

Disclosed herein, in some aspects, are methods that include a cancer. In some aspects, the method further comprises identifying the subject as having a disease state such as cancer, and administering a treatment such as a cancer treatment to the subject.

Disclosed herein, in some embodiments, are methods that include a sample. In some embodiments, the sample comprises a biofluid sample. In some embodiments, the biofluid comprises a blood sample that does not have red blood cells. In some embodiments, the biofluid comprises plasma or serum. In some embodiments, the biofluid comprises a blood sample that is essentially cell-free. In some embodiments, the biofluid is essentially free of red blood cells.

### BRIEF DESCRIPTION OF THE DRAWINGS

FIG. 1 shows exemplary methods useful for data generation and analysis.

FIG. 2 shows a non-limiting example of a computing device; in this case, a device with one or more processors, memory, storage, and a network interface.

FIG. 3A shows lung feasibility study data of 212 subjects with multiple glycoprotein searches to locate glycosylated proteins and the glycan in the samples. 139 glycosylated proteins were identified in both lung feasibility and lung nodule proteomics data sets.

FIG. 3B shows graphs comparing of proteomic data of glycoproteins enriched by contacting with nanoparticles (e.g., NP1, NP2, NP3, NP4, and NP5).

FIG. 4 shows a Proteograph™ plot comparing the significance and difference for enriched peptides of glycoproteins. Individual nanoparticles yielded both complementary and common glycoprotein identifications.

FIG. 5 shows graphs of the number of protein groups and peptides detected across all samples. 5,099 proteins groups and 33,941 peptides across all 5 nanoparticles for the 212 subject samples were detected with a median of 4 peptides per protein for proteins present in >25% of the samples.

FIG. 6 shows a box plot of the number of unique protein groups and the total panel or individual nanoparticle. A median of 1,592 protein groups were detected across all 5 nanoparticles for all 212 subjects in this study. NP5 provided the largest number and most diverse protein groups detected in any of the nanoparticles. Samples were grouped with connecting lines and colored by collections site.

FIG. 7 shows a diagram of PSMs, peptides, and proteins. Peptide-spectral matches (PSMs) corresponding to glycopeptides and glycoproteins were determined from MSFragger searches across the five NP panel from ˜1200 datafiles. A total of 66,511 glyco-PSMs from 877 unique peptides derived from 165 proteins were identified at 1% FDR, with NP1 and NP2 datasets accounting for ˜80% of observed unique N-linked glycopeptides.

FIG. 8 shows a Venn diagram of detected proteins from which glycopeptides were detected in PEAKS or MSFragger glyco searches across nanoparticles NP1 and NP2 (480 files). ˜75% (66/88) of proteins from which glycopeptides were identified via MSFragger and ˜46% (28/61) of proteins with glycopeptides identified via PEAKS were also measured in the Max Quant label free quant (LFQ) search. Little overlap between two algorithms at a glycopeptide level was observed.

FIG. 9 shows a graph of data for detected proteins with top Genecards cancer scores. The detected 5,099 protein groups were mapped to the HPPP database, which illustrated the wide range of reported protein concentrations (8 orders of magnitude) measured with the Proteograph™ nanoparticle technology and timsTOF Pro instrumentation. Additionally, a Genecards6 analysis was performed to determine the cancer associated proteins detected. FIG. 9 also highlighted the top 50 proteins, of which ˜40% had a known plasma concentrations of <10 ng/mL. Glycopeptides in PEAKS searches from 40 of the cancer associated proteins were detected in the study. 42 cancer associated proteins detected also had glycopeptides detected by MSFragger with agreement between the two algorithms on 16 overlapping glycoproteins. Of these 66 unique proteins for which glycopeptides were detected across both algorithms, 53 had multiple previously reported or predicted glycosites from UniProt.

FIGS. 10A-10B show a mass spectrometry diagram of glycosylated or unmodified peptide. MS/MS spectrum from a (FIG. 10A) glycopeptide— FN(HexNAc-4Hex-5NeuAc-1)SSYLQGTNQITGR and (FIG. 10B) its unmodified counterpart, derived from Apolipoprotein-B (APOB), also shortlisted in the Genecard “cancer” search. The spectrum visualized from Byonic (Protein Metrics) following an N-glycan search of a single NP1 file. The APOB glycosite captured by this peptide. Glyco-peptide ions along with N-acetylhexosamine (HexNAc) and N-acetylneuraminic acid (NeuAc) ions were detected in the MS/MS spectrum, consistent with the expected glycopeptide fragments expected to result from collision induced dissociation (CID).

The novel features of the disclosure are set forth with particularity in the appended claims. A better understanding of the features and advantages of the present disclosure will be obtained by reference to the following detailed description that sets forth illustrative embodiments.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

a. Introduction

This disclosure provides non-invasive methods for diagnosing or ruling out the presence of a disease in a subject. Identifying an early-stage disease in a subject can prevent further progression of the disease by leading to earlier initiation of treatment. This can increase patient survival and quality of life. Non-invasive tests can also be used to rule out the presence of a disease, thereby saving subjects from having to undergo invasive testing such as a biopsy, which can be painful and stressful, or may risk damaging the subject. Non-invasive testing can be useful for screening populations and facilitate early diagnosis of the diseases, providing benefits described herein to a wide population.

The method described here may comprise (1) contacting a sample with particles to form a biomolecule corona comprising at least a protein or peptide from the sample. The method may comprise (2) separating at least a protein or peptide from another protein(s) or peptide(s) of the biomolecule corona. The method may comprise (3) combining a protein or peptide with a standard protein or peptide. The method may comprise (4) obtaining a first measurement of the protein or peptide of the biomolecule corona. The method may comprise the step of (1); (2); (3) and (4). The method may comprise the step of (3), (1), (2), and (4); wherein (3) may comprise combining a standard protein or peptide with the sample; wherein the biomolecule corona of (1) may comprise at least a protein/peptide from the sample and/or at least one standard protein/peptide. The method may comprise the step of (1), (3), (2); and (4), wherein (3) comprises combining the sample and/or the biomolecule corona with the standard protein or peptide; wherein (2) may comprise separating at least a protein or peptide and at least a standard protein or peptide from another protein(s) or peptide(s) of the biomolecule corona. In some cases, the method may comprise repeating any of the steps of the methods described herein. In some cases, any step of the methods may be repeated at least 1, 2, 3, 4, 5 or more times. In some cases, (4) may be performed prior to or subsequent to any of the other steps; wherein (4) obtaining a first measurement of the protein or peptide of the sample or corona.

The method may comprise (5) obtaining a data set comprising at least a protein or peptide from a biomolecule corona. The data set may be generated subsequent to performing (1), (2), (3), (4), or any combination thereof. In some case, the data may be generated without performing any of (1)-(4). The method described here may further comprise (6) determining whether a sample is associated with a disease or risk of disease or the subject in which the sample originates from or is obtained from has the disease or the risk of the disease. In some cases, (6) may be carried out subsequent to (4) or (5). In some cases, (6) may comprise applying an algorithm to the data set to identify whether the sample is associated with the disease or risk of disease or the subject in which the sample originates from or is obtained from has the disease or the risk of the disease. In some cases, (6) may comprise determine whether a level of a biomolecule is different from a threshold level, wherein the difference indicates that the disease or the risk thereof. The threshold level may comprise any threshold levels described herein.

In some cases, (2) may comprise additional enrichment of the protein/peptides as described herein. The proteins or peptides or internal proteins or peptides may comprise a protein or peptide that has a post-translational modification. In some cases, the post-translational modification may comprise glycosylation (e.g., protein or peptide or internal thereof may be glycosylated or is a glycoprotein or glycopeptide).

In some cases, priori to (5), the method may further comprise (7) releasing a glycan moiety from a protein or peptide. In this case, (5) of the method may comprise obtaining a data set comprising at least a glycan moiety.

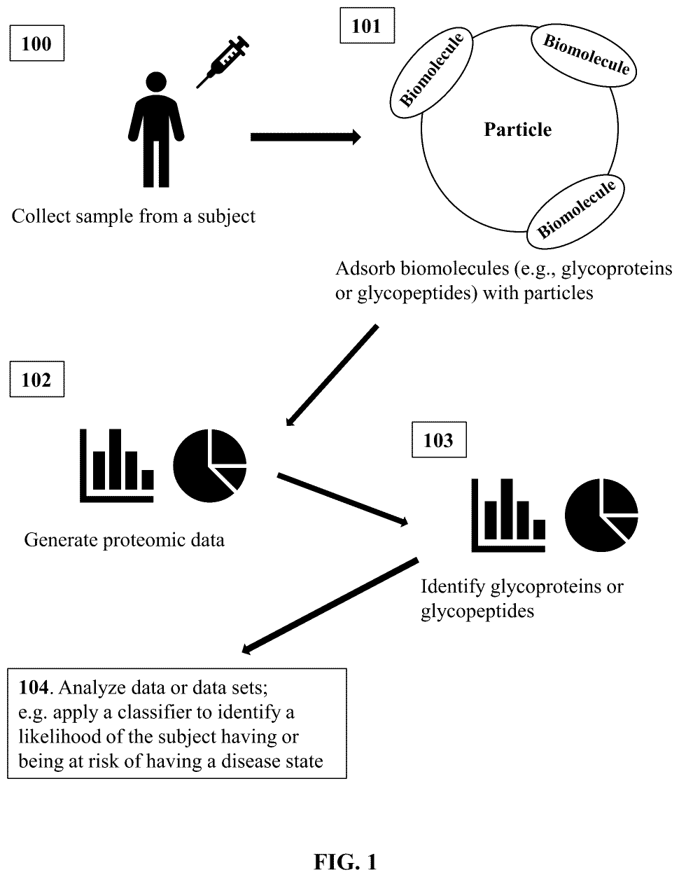

FIG. 1 illustrates a non-limiting example of methods for predicting whether a subject has or is at risk of developing a disease based on assaying and analyzing a biofluid sample obtained from the subject (100). The biofluid sample can be any one of or any combination of the biofluids described herein. The sample can be contacted with particle described herein to obtain adsorbed biomolecules (101). The adsorbed biomolecules may be analyzed to generate data (102) protein or peptide data. The data may be analyzed to identify glycoproteins or glycopeptides (103). The data may be analyzed to identify a likelihood of the subject having or at risk of having the disease (104).

The methods described herein include generating or obtaining proteomic data and using the proteomic data to make a prediction about whether a subject has or does not have a disease. Various ways of combining or analyzing proteomic data are described. Uses of the proteomic data and disease assessment are further elaborated.

The methods described herein may be used to predict or identify a disease state. A disease state may include a disease or disorder such as cancer. Examples of cancer include lung cancer, colon cancer, pancreatic cancer, liver cancer, ovarian cancer, breast cancer, prostate cancer, melanoma, bladder cancer, lymphoma, leukemia, renal cancer, or uterine cancer. An example of lung cancer is non-small cell lung cancer (NSCLC). The cancer may be at an early stage or a late stage. A disease may include a disorder. A disease state may include having a comorbidity related to a disease or disorder. A reference to whether a subject has a disease state or not may include the subject being healthy. A healthy state may exclude a disease state. For example, a healthy state may exclude having cancer. A disease state may exclude being healthy.

b. Samples and Sample Preparation

Data such as protein data may be generated from a sample of a subject. The sample may be a biofluid sample. A sample may include a tissue or cell homogenate. A sample may include a biofluid. A biofluid may comprise a body fluid. A body fluid may comprise an extracellular body fluid. A biofluid may be cell-free or substantially cell-free. To obtain a cell-free or substantially cell-free biofluid sample, a biofluid may undergo a sample preparation method such as centrifugation and pellet removal.

A body fluid may comprise amniotic fluid, aqueous humor, ascites, bile, bone marrow, breast milk, broncheoalveolar lavage fluid, bronchopulmonary aspirates or other lavage fluids, cerebrospinal fluid, cerumen, chyle, chyme, Cowper's fluid or pre-ejaculatory fluid, cyst fluid, fecal matter, ejaculate, interstitial fluid, intravascular fluid, lavage fluids from sinus cavities, lymph, lymphatic fluid, menses, milk, mucosal secretion, mucus, pancreatic juice, pericardial fluid, pleural and peritoneal fluid, pus, saliva, sebum, semen, semen (including prostatic fluid), sputum, stool water, sweat, synovial fluid, tears, transcellular fluid, urine, vaginal fluid, vaginal secretions, vomit, or a combination thereof. A biofluid can comprise blood, serum, or plasma. A biofluid can comprise blood. A biofluid can comprise plasma. A biofluid can comprise serum.

A biofluid sample may be obtained from a subject. For example, a blood, serum, or plasma sample may be obtained from a subject by a blood draw. Other ways of obtaining biofluid samples include aspiration or swabbing.

A non-biofluid sample may be obtained from a subject. For example, a sample may include a tissue sample. Some examples of organs or tissues that may be sampled include lung, colon, pancreatic, liver, or ovarian tissue. The sample may include a mass taken from the organ or tissue of the subject. The mass may be suspected of being cancerous. The mass may include a nodule. The mass may include a cyst. The nodule or cyst may be identified by a physician as at a high risk or low risk of being cancerous. The sample may include a cell sample. The sample may include a homogenate of a cell or tissue. The sample may include a supernatant of a centrifuged homogenate of a cell or tissue.

The sample can be obtained invasively or non-invasively. Invasive sampling may comprise extracting a sample from a subject that requires an introduction of an instrument into the body of the subject or results in the exposure of an internal body fluid or cavity. Invasive sampling may result in a wound, injury, or flesh opening in the subject (that the sample is extracted from). Non-invasive sampling may comprise extracting a sample from a subject without an introduction of an instrument into the body of the subject or resulting in the exposure of an internal body fluid or cavity. Non-invasive sampling may not result in sampling may result in a wound, injury, or flesh opening in the subject (that the sample is extracted from). Because non-invasive sampling does not cause an injury to a subject being sampled, non-invasive sampling may facilitate multiple samplings. In some cases, non-invasive sampling may reduce or minimize adverse effects in the subject being sampled. Such an adverse effect may comprise infection or injury.

The sample can be obtained from the subject during any phase of a screening procedure, such as before, during, or after a stage. The sample can be obtained before or during a stage where the subject is a candidate for a biopsy, for early detection of a disease. The sample can be obtained before or during a non-invasive work-up, an invasive work-up, treatment, a monitoring stage.

Data may be generated from a single sample, or from multiple samples. Data from multiple samples may be obtained from the same subject. In some cases, different data types are obtained from samples collected differently or in separate containers. A sample may be collected in a container that includes one or more reagents such as a preservation reagent or a biomolecule isolation reagent. Some examples of reagents include heparin, ethylenediaminetetraacetic acid (EDTA), citrate, an anti-lysis agent, or a combination of reagents. Samples from a subject may be collected in multiple containers that include different reagents, such as for preserving or isolating separate types of biomolecules. A sample may be collected in a container that does not include any reagent in the container. The samples may be collected at the same time (e.g., same hour or day), or at different times. A sample may be frozen, refrigerated, heated, or kept at room temperature.

Provided herein are methods to process a sample described herein. In some cases, the method comprises additionally enriching (or additional enrichment of) at least a biomolecule (or a set of) from the sample. The additionally enrichment or additional enrichment is different from using the particle to enrich a biomolecule as described herein. In some cases, the additional enrichment of a biomolecule may be carried out prior to the enrichment using the particle. In some cases, the additionally enrichment of a biomolecule may be carried out during the enrichment using the particle. In some cases, the additionally enrichment of a biomolecule may be carried out subsequent to the enrichment using the particle. In some cases, the additionally enrichment of a biomolecule may be carried out subsequent to releasing the biomolecule from the biomolecule corona. The methods for releasing the biomolecule from the biomolecule corona (or particle) is described elsewhere in this disclosure. In some cases, the additionally enrichment of a biomolecule may be carried out during the biomolecule is released from the biomolecule corona. In some cases, the additionally enrichment of a biomolecule may be prior out subsequent to releasing the biomolecule from the biomolecule corona. In some cases, the additional enrichment of a biomolecule may be carried out in a sample; or a solution comprising the sample, particle, biomolecule corona, biomolecules, or a combination thereof.

In some instances, the additional enrichment may comprise enriching a subset of biomolecules from other biomolecules. The subset of biomolecules may comprise similar physiochemical properties. In some cases, the additional enrichment may comprise enriching a subset of proteins/peptides from other biomolecules. In some cases, the additional enrichment may comprise enriching a subset of proteins/peptides from other proteins/peptides. In some cases, the additional enrichment may comprise enriching glycoproteins/glycopeptides from other biomolecules or proteins/peptides. In some cases, the additional enrichment may comprise enriching a subset of glycoproteins/glycopeptides from other glycoproteins/glycopeptides.

The additional enrichment has an beneficial advantage in which the enrichment of glycoproteins or glycopeptides for measurements, determination, or analysis using the methods described herein. When processing a sample comprising proteins and peptides using enzymatic digestion (such as trypsin) for measurements (such as by mass spectroscopy), glycopeptides are often less abundant than other tryptic peptides, making identification of the glycopeptides challenging. The additional enrichment may increase the numbers of glycoproteins or glycopeptides identified, using and/or combining the methods or method steps described herein.

Some aspects include separating glycans from glycoproteins or glycopeptides. Some aspects include separating a glycan from a glycoprotein. Some aspects include separating a glycan from a glycopeptide.

In some cases, the additional enrichment may comprise using chromatography. Chromatography may comprise liquid chromatography, gas chromatography, column chromatography, ion-exchange chromatography, gel-permeation (molecular sieve) chromatography, affinity chromatography, paper chromatography, thin-layer chromatography, dye-ligand chromatography, hydrophobic interaction chromatography, pseudoaffinity chromatography, or a combination thereof. Chromatography may comprise liquid chromatography. Chromatography may comprise gas chromatography. Chromatography may comprise column chromatography. Chromatography may comprise ion-exchange chromatography. Chromatography may comprise gel-permeation (molecular sieve) chromatography. Chromatography may comprise affinity chromatography. Chromatography may comprise paper chromatography. Chromatography may comprise thin-layer chromatography. Chromatography may comprise dye-ligand chromatography. Chromatography may comprise hydrophobic interaction chromatography. Chromatography may comprise pseudoaffinity chromatography. Chromatography may comprise liquid chromatography and gas chromatography. Chromatography may comprise liquid chromatography and column chromatography. Chromatography may comprise liquid chromatography and ion-exchange chromatography. Chromatography may comprise liquid chromatography and gel-permeation (molecular sieve) chromatography. Chromatography may comprise liquid chromatography and affinity chromatography. Chromatography may comprise liquid chromatography and paper chromatography. Chromatography may comprise liquid chromatography and thin-layer chromatography. Chromatography may comprise liquid chromatography and dye-ligand chromatography. Chromatography may comprise liquid chromatography and hydrophobic interaction chromatography. Chromatography may comprise liquid chromatography and pseudoaffinity chromatography.

In the methods described herein, column chromatography may comprise using a column to enrich the target biomolecules (proteins/peptides or glycosylated thereof) based on the size, shape, and/or net charge of the target biomolecules being enriched. Ion chromatography may comprise using electrostatic interactions between charged biomolecules and solid support material (matrix). Gel-permeation chromatography may comprise using dextran containing materials to separate target biomolecules based on their differences in molecular sizes. Affinity chromatography may comprise a ligand that can bind the target biomolecules. Paper chromatography may comprise using a layer of cellulose saturated with water and enrich the target biomolecules based on their mobility in the cellulose. Thing-layer chromatography may comprise using solid adsorbent substance to enrich the target biomolecules. Gas-chromatography may comprise vaporizing the biomolecules and enriching the target biomolecules based on the dispersion between the gaseous mobile phase and a liquid stationary phase adsorbed onto the surface of an inert solid material. Liquid chromatography may comprise injecting a liquid/solution comprising biomolecules into a stream of solvent (mobile phase) flowing through a column packed with a separation medium (stationary phase) to enrich the target biomolecules.

Liquid chromatography may comprise high performance liquid chromatography (HPLC). HPLC may comprise mobile phase at an atmospheric pressure at a high flow rate. The atmospheric pressure of the HPLC may be measure by Pascal (Pa). In some cases, the atmospheric pressure of the HPLC may be at least about 1 Pa, 2 Pa, 5 Pa, 10 Pa, 20 Pa, 50 Pa, 100 Pa, 200 Pa, 500 Pa, 1000 Pa, 2000 Pa, 5000 Pa, 10000 Pa or more. In some cases, the atmospheric pressure of the HPLC may be at least about 1 Pa, 2 Pa, 5 Pa, 10 Pa, 20 Pa, 50 Pa, 100 Pa, 200 Pa, 500 Pa, 1000 Pa, 2000 Pa, 5000 Pa, or 10000 Pa. In some cases, the flow rate of the mobile phase in the HPLC may be at least about 1 micrometer (μm) per second, 2 μm per second, 5 μm per second, 10 μm per second, 20 μm per second, 50 μm per second, 50 μm per second, 100 μm per second, 200 μm per second, 500 μm per second, 1 millimeter (mm) per second, 2 mm per second, 5 mm per second, 10 mm per second, 20 mm per second, 50 mm per second, 50 mm per second, 100 mm per second, 200 mm per second, 500 mm per second, 500 mm per second, 1000 mm per second or more. In some cases, the flow rate of the mobile phase in the HPLC may be at most about 1 micrometer (μm) per second, 2 μm per second, 5 μm per second, 10 μm per second, 20 μm per second, 50 μm per second, 50 μm per second, 100 μm per second, 200 μm per second, 500 μm per second, 1 millimeter (mm) per second, 2 mm per second, 5 mm per second, 10 mm per second, 20 mm per second, 50 mm per second, 50 mm per second, 100 mm per second, 200 mm per second, 500 mm per second, 500 mm per second, or 1000 mm per second.

Some aspects comprise enriching glycoproteins or glycopeptides. Some aspects comprise separating glycoproteins or glycopeptides from other proteins or peptides. Some aspects comprise, following nanoparticle capture of glycoproteins or glycopeptides, enriching the glycoproteins or glycopeptides, or separating the glycoproteins or glycopeptides from other proteins or peptides. Some aspects include enriching glycoproteins or glycopeptides after capturing the glycoproteins or glycopeptides. Some aspects include separating glycoproteins or glycopeptides from other proteins or peptides, after capturing the glycoproteins or glycopeptides and proteins or peptides (e.g. using particles). The glycoproteins or glycopeptides may comprise glycoproteins. The glycoproteins or glycopeptides may comprise glycopeptides.

An additional enrichment may comprise liquid chromatography. The liquid chromatography may comprise hydrophilic interaction liquid chromatography (HILIC), electrostatic repulsion liquid chromatography (ERLIC), high performance liquid chromatography (HPLC), supercritical fluid chromatography (SFC), Reverse phase liquid chromatography (RP-LC), or a combination thereof. The additional enrichment may comprise HILIC. The additional enrichment may comprise ERLIC. The additional enrichment may comprise SFC. The additional enrichment may comprise HPLC. The additional enrichment may comprise RP-LC. The additional enrichment may comprise at least two of the HILIC, the ERLIC, the HPLC, the SFC, and the RP-LC. The additional enrichment may comprise HILIC and ERLIC. The additional enrichment may comprise the HILIC and the HPLC. The additional enrichment may comprise the HILIC and the SFC. The additional enrichment may comprise the HILIC and the RP-LC. The additional enrichment may comprise the ERLIC and the HPLC. The additional enrichment may comprise the ERLIC and the SFC. The additional enrichment may comprise the ERLIC and the RP-LC. The additional enrichment may comprise the HPLC and the SFC. The additional enrichment may comprise the HPLC and the RP-LC. The additional enrichment may comprise the SFC and the RP-LC. HILIC may comprise using the polar nature of the sugar moiety (such as glycan moiety) and the solid phase (such as those comprising cellulose materials) to enrich the polar glycoproteins or glycopeptides from the other proteins or peptides (such as tryptic peptides). Additionally, HILIC may also use hydrogen bonds between polar glycoproteins or glycopeptides and the solid phase to enrich the glycoproteins or glycopeptides from other hydrophobic biomolecules. ERLIC may comprise using electrostatic interactions between the positively charged groups (such as polyethylene mine) bound (for example, covalently) to a stationary phase (such as using modified silica bead). The positively charged group repels biomolecules with positive charges, such as positively charged proteins or peptide; and enriches negatively charged biomolecules, such as glycoproteins or glycopeptides (for example, sugar moieties are negatively charged). Additionally, ERLIC may also use hydrogen bonds between polar glycoproteins or glycopeptides and the solid phase to enrich the glycoproteins or glycopeptides from other hydrophobic biomolecules. Parameters of HILIC and/or ERLIC may be those disclosed in Zacharias et al., J Proteome Res. 2016 Oct. 7; 15(10): 3624-3634, which is herein incorporated by reference in its entirety. RP-LC may comprise a hydrophobic stationary phase. For example, hydrophobic molecules covalently bonded to the stationary phase may be used as the hydrophobic stationary phase during the chromatography. In this case, the more hydrophobic a molecule is, the more likely it will bind to the stationary phase. Hydrophobic molecules in the mobile phases may be adsorbed or bind to the hydrophobic stationary phase, leaving the less hydrophobic molecules (such as protein/polypeptides or glycosylated versions thereof) in the polar hydrophilic mobile phase to pass through the stationary phase. SFC may comprise using a supercritical fluid (e.g., a substance at temperature and pressure above a critical point). The mobile phase may have liquid properties to dissolve molecules and gaseous bonding properties (e.g., chromatographic properties) and kinetics. SFC may facilitate separation of low to moderate molecular weight molecules. In some cases, the supercritical fluid may comprise carbon dioxide.

In some cases, provided herein are devices performing any chromatography described herein. For examples, the device may perform HPLC. The device may perform HILIC. The device may perform ERLIC. The device may perform SFC. The device may perform RP-LC.

The methods descried herein may use an enzyme to digest biomolecules (within a sample or subsequent to the biomolecules contacted with a particle or released from biomolecule coronas). The additional enrichment may be carried subsequent to the enzymatic digestion. The additional enrichment may be carried during the enzymatic digestion. The additional enrichment may be carried prior to the enzymatic digestion. The enzyme may be trypsin. In some cases, the digestion may be non-enzymatic (for example, using a chemical that is not an enzyme to digest the biomolecules).

The additional enrichment may also comprise using an affinity reagent such as an antibody to enrich (e.g., immunoprecipitate) the target biomolecules. The enrichment may be performed before spiking the sample with the internal standards described herein, and may include adhering biomolecules to the affinity reagent, centrifuging or concentrating the affinity reagents adhered to the biomolecules, removing or separating excess sample or other biomolecules not to be measured from the affinity reagents adhered to the biomolecules, and eluting the biomolecules from the affinity reagents. Use of affinity reagents in this way may be used to enrich for specific types of biomolecules or pathways. For example, proteins with a particular post-translational modification (PTM), or proteins of a particular molecular pathway may be enriched through the use of one or more affinity reagents specific for that post-translational modification or molecular pathway.

The methods described herein can comprise separating biomolecules. Separation of biomolecules and enrichments/additional enrichments of biomolecules may use the same techniques/steps described herein, since separating two molecules may be used to enrich either one of the two molecules. For examples, biomolecules within the sample or a solution comprising the samples or the biomolecules (with or without the internal standards described herein) may be separated. The separating or separation may comprise gel electrophoresis, liquid chromatography described herein, or solid phase extraction (SPE). The liquid chromatography may comprise HPLC, hydrophilic interaction liquid chromatography (HILIC), electrostatic repulsion liquid chromatography (ERLIC), supercritical fluid chromatography (SFC), Reverse phase liquid chromatography (RP-LC), or a combination thereof. The liquid chromatography may comprise HILIC. The liquid chromatography may comprise ERLIC. The liquid chromatography may comprise HPLC. The liquid chromatography may comprise SFC. The liquid chromatography may comprise RP-LC. The liquid chromatography may comprise at least two of the HILIC, the ERLIC, the HPLC, the SFC, and the RP-LC. The liquid chromatography may comprise HILIC and ERLIC. The liquid chromatography may comprise the HILIC and the HPLC. The liquid chromatography may comprise the HILIC and the SFC. The liquid chromatography may comprise the HILIC and the RP-LC. The liquid chromatography may comprise the ERLIC and the HPLC. The liquid chromatography may comprise the ERLIC and the SFC. The liquid chromatography may comprise the ERLIC and the RP-LC. The liquid chromatography may comprise the HPLC and the SFC. The liquid chromatography may comprise the HPLC and the RP-LC. The liquid chromatography may comprise the SFC and the RP-LC. The gel electrophoresis may comprise one-dimensional gel electrophoresis. The gel electrophoresis may comprise multi-dimensional (or high dimensional) gel electrophoresis. The multi-dimensional gel electrophoresis may comprise at least 2, 3, 4 or more dimensions. The gel electrophoresis may comprise 2-dimensional gel electrophoresis. The gel electrophoresis may comprise 3-dimensional gel electrophoresis. The gel electrophoresis may comprise 4-dimensional gel electrophoresis. The gel electrophoresis may comprise more than 4 dimensions. When referring to gel-electrophoresis, a dimension refers to a factor of the biomolecules being used in the gel-electrophoresis to separate the biomolecules. For example, factors of the biomolecules being used in the gel-electrophoresis may comprise the molecular weights, isoelectric points, charges, ionic strength, degrees of hydrophobicity/hydrophilicity, or a combination thereof of the biomolecules. A multi-dimensional gel electrophoresis may use molecular weights and isoelectric points of the biomolecules as separation factors. A multi-dimensional gel electrophoresis may use molecular weights and charges as separation factors. A multi-dimensional gel electrophoresis may use molecular weights and ionic strength as separation factors. A multi-dimensional gel electrophoresis may use molecular weights and degrees of hydrophobicity/hydrophilicity as separation factors. A multi-dimensional gel electrophoresis may use isoelectric points and charges as separation factors. A multi-dimensional gel electrophoresis may use isoelectric points and ionic strength as separation factors. A multi-dimensional gel electrophoresis may use isoelectric points and degrees of hydrophobicity/hydrophilicity as separation factors. A multi-dimensional gel electrophoresis may use charges and ionic strength as separation factors. A multi-dimensional gel electrophoresis may use charges and degrees of hydrophobicity/hydrophilicity as separation factors. A multi-dimensional gel electrophoresis may use ionic strength and degrees of hydrophobicity/hydrophilicity as separation factors. A multi-dimensional gel electrophoresis may use any 3 or 4 of the weights, isoelectric points, charges, ionic strength, or degrees of hydrophobicity/hydrophilicity as separation factors. A multi-dimensional gel electrophoresis may use the weights, isoelectric points, charges, ionic strength, and degrees of hydrophobicity/hydrophilicity as separation factors. When running a multi-dimensional gel electrophoresis, separation of each factor of the biomolecules may be carried out simultaneously or sequentially. SPE may comprise solid-liquid extraction of biomolecules that are dissolved or suspended in a liquid mobile phase from other molecules using the physical (e.g., size, shape, or mobility of the biomolecules) and chemical properties (e.g., bonding properties) of the biomolecules. SPE may comprise chromatography described herein. In other cases, SPE may not comprise a continuous mobile phase, as opposed to liquid chromatography.

In some instances, the method comprising using an enzyme to release a glycan moiety from a biomolecule (such as glycoprotein/glycopeptide) The biomolecule may be adsorbed onto the surface of the particle or part of a biomolecule corona. In other cases, the enzyme may be used to release the glycan from a biomolecule not adsorbed on the particle or part of the biomolecule corona. In some cases, the enzyme may be added subsequent to the biomolecule is released from the particle or biomolecule corona. For example, the enzyme may comprise an enzyme that can break a glycosidic bond. A glycosidic bond may comprise an alpha-glycosidic bond or a beta glycosidic bond. An alpha-glycosidic bond may comprise the bond in which both carbons of the bond have the same stereochemistry. An alpha-glycosidic bond may comprise alpha-1,4 glycosidic bond or alpha-1,6 glycosidic bond. A beta-glycosidic bond may comprise the bond in which the two carbons of the bond have different stereochemistry. A beta-glycosidic bond may comprise beta-1,4 glycosidic bond. A glycosidic bond may comprise C-, N-, O-, or S-glycosidic bonds (see glycosylation described herein). In some cases, the enzyme for breaking a glycosidic bond may comprise amylase, glycoside hydrolase, amylase, maltase, isomaltase, cellulase, amidase (N-glycosidase F (PNGase F) or PNGase A), endoglycosidase (endoglycosidase D or endoglycosidase H or endoglycosidase F), O-glycanase, or any combination thereof. In some cases, the enzyme for breaking a glycosidic bond may comprise amylase. In some cases, the enzyme for breaking a glycosidic bond may comprise glycoside hydrolase. In some cases, the enzyme for breaking a glycosidic bond may comprise amylase. In some cases, the enzyme for breaking a glycosidic bond may comprise maltase. In some cases, the enzyme for breaking a glycosidic bond may comprise isomaltase. In some cases, the enzyme for breaking a glycosidic bond may comprise cellulase. In some cases, the enzyme for breaking a glycosidic bond may comprise PNGase F. In some cases, the enzyme for breaking a glycosidic bond may comprise endoglycosidase. In some cases, the enzyme for breaking a glycosidic bond may comprise endoglycosidase D. In some cases, the enzyme for breaking a glycosidic bond may comprise endoglycosidase H. In some cases, the enzyme for breaking a glycosidic bond may comprise endoglycosidase F. In some cases, the enzyme for breaking a glycosidic bond may comprise O-glycanase. In some cases, a glycosidic bond may be broken by chemical reductive β-elimination.

A glycan moiety may comprise an N-glycan or O-glycan. A glycan moiety may comprise at least a saccharide moiety. A saccharide moiety of the N-glycan may comprise mannose, glucose, N-acetylglucosamines, galactose, sialic acid, or a combination thereof. During the synthesis of the N-glycan for N-glycosylation (described elsewhere in this disclosure), in the cytoplasmic side of the ER, two UDP-GlcNAC (glucosamines) are attached to a dolichol molecule. Five-GDP-mannoses are attached to the GlcNAC. Subsequently, the glycan may then be translocated into the lumen side of the ER. Subsequent saccharides (such as 4 mannose and 3 glucose) are added to the glycan. Subsequently, the glycan is transferred to the protein/peptide, forming glycoprotein/glycopeptide (N-linked glycosylated). Subsequently, the glycan is processed at least via ER and Golgi for trimming, addition, and/or branching using various saccharides described herein, generating multitude of glycan types. Methods described herein can determine the identity and quantity of the multitude of glycan types. For example, an N-glycan may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 1000 or more saccharide moieties. An N-glycan may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, or 1000 saccharide moieties. In some cases, N-glycan may be branched or linear. In some cases, N-glycan may comprise high-moose types (two N-acetylglucosamines with various mannoses), complex oligosaccharide type (any number of saccharides including more than two N-acetylglucosamines), or hybrid oligosaccharide (with a mannose on one side of the branch and a N-acetylglucosamine on another side of the branch).

A saccharide moiety of the O-glycan may comprise N-acetyl-galactosamine (GalNAc), galactose (GAL), N-acetyl-glucosamine, sialic acid, N-acetylneuraminic acid, fucose, or a combination thereof. During the synthesis of the O-glycan for O-glycosylation (described elsewhere in this disclosure), N-acetylgalactosamine is first attached to a serine or threonine of a protein/peptide via N-acetylgalactosamine transferase. Additional saccharides can be added to the N-acetyl-galactosamine attached to the proteins. For example, an O-glycan may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 1000 or more saccharide moieties. An O-glycan may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, or 1000 saccharide moieties. O-glycan may comprise various Cores. Core 1 may be generated by the addition of galactose. Core 2 may be generated by the addition of N-acetyl-glucosamine to the N-acetyl-galactosamine of Core 1. Core 3 may be generated by the addition of a single N-acetyl-glucosamine to the original N-acetyl-galactosamine. Core 4 may be generated by the addition of a second N-acetyl-glucosamine to Core. Cores 3, 4, and 6 are β-GlcNAcylated on C3-hydroxyl (C3-OH) and/or C6-OH of the initiating GalNAc. Cores 5, 7, and 8 contain α-linked extensions (α1-3GalNAc, α1-6GalNAc, and α1-3Gal, respectively). O-glycan may also comprise other cores not comprising Cores 1-8. In some cases, O-glycan may be branched or linear.

When releasing a glycan moiety from the glycoprotein/glycopeptide using the enzyme described herein, the enzymatic reaction may be conducted in heavy water. In such case, the glycosylated site (de-glycosylated site) in which the glycan moiety is released by the enzyme can be labeled with an isotope of the heavy water. The method may then generate a labeled de-glycosylated glycoprotein/glycopeptide. The heavy water may comprise deuterium. The deuterium may be used as the isotope to label the de-glycosylated site.

In other cases, when using the enzyme described herein (such as PNGase F or Endo F/Endo H), the de-N-glycosylated peptides can be mass-tagged by conversion of asparagine to aspartic acid (a +1 Dalton change in the peptide mass) within the consensus sequon (for example, PNGase F can cleave the glycosidic bond between the core GlcNAc and the asparagine residue in the NXT/S sequence (X≠proline, N=asparagine, T=threonine, S=serine) or by retention of a GlcNAc(±Fuc) at the asparagine. Additionally, the sites with the asparagine to aspartic acid in peptides can also be identified as the glycosylation sites.

For releasing a glycan moiety from a glycoprotein/glycopeptide, the enzyme may contact the glycoprotein/glycopeptide for at least about 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours or more. The enzyme may contact the glycoprotein/glycopeptide for at most about 1 second, 2 seconds, 3 seconds, 4 seconds, 5 seconds, 6 seconds, 7 seconds, 8 seconds, 9 seconds, or 10 seconds The enzyme may contact the glycoprotein/glycopeptide at least about −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 50° C., 60° C. or more. The enzyme may contact the glycoprotein/glycopeptide at most about −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 50° C., or 60° C.

Subsequent to the release of a glycan moiety from a protein/peptide (or glycoprotein/glycopeptide), the glycan moiety may be used as the biomolecule for generating the data or determining if a subject has a disease condition or a risk thereof (or a sample or a solution comprising the sample, or the protein/peptide (or glycoprotein/glycopeptide) is associated with the disease condition or risk thereof. In some cases, the method comprising using the enzyme to break down a glycosidic bond may release the glycan moiety from the glycoprotein/glycopeptide and analyze the glycan moiety using the mass spectroscopy to generate the data described herein. The glycan moiety analysis data may be referred to as glycomics.

c. Particles and Uses Thereof

The methods described herein may comprises using a particle to generate a biomolecule corona. Data may be obtained using a particle. The particle may be a non-naturally occurring particle, a naturally occurring particle or a combination thereof. The particle may be a non-naturally occurring particle. The particle may be a naturally occurring particle.

Biological samples may be contacted with particles, for example, prior to generating data. The data described herein may be generated using methods that use the particles. For example, a method may include contacting a sample with particles such that the particles adsorb biomolecules. The particles may attract different sets of biomolecules than would normally be difficult to measure accurately by performing omic measurements directly on the sample. For example, a dominant biomolecule may make up a large percentage of certain type of biomolecules (e.g., proteins, transcripts, genetic material, lipids, or metabolites) in a sample. By adsorbing biomolecules to particles prior to analyzing them, a subset of biomolecules may be obtained that does not include the dominant biomolecule. Removing dominant biomolecules (e.g., biomolecules that make up a majority of a biological sample) in this way may increase the accuracy of biomolecule measurements and sensitivity of an analysis using those measurements.

Adsorption may comprise a physical phenomenon in which a molecule adheres to a surface of another molecule. When a biomolecule described herein is adsorbed to the particle described herein, the biomolecule may be adhered onto the surface of the particle without penetrating through the surface of the particle. Adsorption may be mediated by surface tension. In a solution, all the bonding requirements (including ionic, covalent, or metallic bonds) of the biomolecules may be occupied by the other biomolecules. When a solution (such as a sample described herein) comprises the particle described herein, the surface of the particle may provide additional bonding properties to the biomolecules, thereby allowing at least a subset of the biomolecules in the sample to bond with the particles in which the bonding between the particle/subset of biomolecules are stronger or higher than those between the subset of biomolecules and other biomolecules in the solution (e.g., these biomolecules are adsorbed onto the surface of the particle). The adsorption of the biomolecules onto the surface of the particle may be mediated by various bonds, such as physisorption (such as van der Waals force) or chemisorption (e.g., covalent bond, metallic bond, and/or ionic bonds). By using particles with physiochemically distinct properties (such as those described herein), various particles can adsorb various subsets of biomolecules (such as proteins/peptides) that has various matching bonding properties (e.g., the bonding force between the subsets of biomolecules and the particles' surface is higher or stronger than those between the subsets of biomolecules and other biomolecules in the solution). This way, by using the particles described herein, various biomolecules with similar physiochemical properties can be isolated from a sample.

Physiochemical properties of particles or biomolecules may refer to various bonding properties presented onto the surface of the particles or biomolecules. Physiochemical properties may be determined by the atomic, molecular, or chemical make-up of the particles or biomolecules; the factors present (such as pH, atmospheric pressure, density, ionic strength, temperature, or a combination thereof) of the environment (such as the solution or sample) in which the particles or biomolecules reside within or are in contact with; or a combination thereof. Various particles described herein may each have a same (or substantially the same) physiochemical property. For example, two particles may have the same physiochemical properties if they are to adsorb the same or substantially the same set of biomolecules. For example, two particles may have the same physiochemical properties if equal to or at least about 70%, 80%, 90%, 99% or more of all biomolecules they adsorb are the same biomolecules. Various particles described herein may have physiochemically distinct (different physiochemical properties) properties. For example, two particles may have physiochemically distinct properties of less than 70%, 60%, 50%, 40%, 30%, 20%, 10%, 9%, 8%, 7%, 6%, 5%, 4%, 3%, 2%, 1%, or 0% of all biomolecules they adsorb are the same biomolecules. The particles described herein may comprise various chemical or physical make-ups as described herein and can comprise physiochemically distinct properties.

The particles may be useful in a method that include contacting a biological sample with particles, thereby adsorbing endogenous biomolecules of the biological sample to the particles; and combining the biological sample or the adsorbed endogenous biomolecules with reference biomolecules (e.g., internal standards) of the biomolecules. Endogenous biomolecules may comprise molecules present in a biological sample without or before addition of other molecules.

Examples of biomolecules that may be adsorbed to particles include proteins, transcripts, genetic material, or metabolites. The adsorbed biomolecules may make up a biomolecule corona around the particle (e.g., the biomolecule corona may comprise the biomolecules adsorbed onto the particle). The adsorbed metabolites may be measured or identified in generating a data set. The adsorbed metabolites may be measured or identified in generating data such as proteomic data.

Particles can be made from various materials. Such materials may include metals, magnetic particles, polymers, or lipids. A particle may be made from a combination of materials. A particle may comprise layers of different materials. The different materials may have different properties. A particle may include a core comprising one material and be coated with another material. The core and the coating may have different properties.

Disclosed herein in some aspects, are methods that include contacting a sample from a subject with particles to form a biomolecule corona comprising glycoproteins or glycopeptides adsorbed to the particles; and releasing at least one glycan moiety from the glycoproteins or glycopeptides adsorbed to the particles.

In some cases, the particle may adsorb at least a protein or peptide (comprising a standard protein or peptide) to generate a biomolecule corona. A biomolecule corona may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, 5000, 10,000, 15,000, or 20,000 distinct proteins or peptides (e.g., including glycoproteins or glycopeptides), or a range of proteins or peptides defined by any two of the aforementioned integers. The range may include at least any of the aforementioned numbers of proteins or peptides (e.g., including glycoproteins or glycopeptides). In some cases, the range may include no more than any of the aforementioned numbers of proteins or peptides (e.g., including glycoproteins or glycopeptides). A biomolecule corona may comprise 1-5000 distinct proteins/peptides. A biomolecule corona may comprise 2-2000 distinct proteins/peptides. A biomolecule corona may comprise 3-1000 distinct proteins/peptides. A biomolecule corona may comprise 4-500 distinct proteins/peptides. A biomolecule corona may comprise 5-500 distinct proteins/peptides. A biomolecule corona may comprise 6-500 distinct proteins/peptides. A biomolecule corona may comprise 7-500 distinct proteins/peptides. A biomolecule corona may comprise 8-500 distinct proteins/peptides. A biomolecule corona may comprise 9-500 distinct proteins/peptides. A biomolecule corona may comprise 10-500 distinct proteins/peptides. A biomolecule corona may comprise 1-400 distinct proteins/peptides. A biomolecule corona may comprise 2-400 distinct proteins/peptides. A biomolecule corona may comprise 3-400 distinct proteins/peptides. A biomolecule corona may comprise 4-400 distinct proteins/peptides. A biomolecule corona may comprise 5-400 distinct proteins/peptides. A biomolecule corona may comprise 6-400 distinct proteins/peptides. A biomolecule corona may comprise 7-400 distinct proteins/peptides. A biomolecule corona may comprise 8-400 distinct proteins/peptides. A biomolecule corona may comprise 9-400 distinct proteins/peptides. A biomolecule corona may comprise 10-400 distinct proteins/peptides. A biomolecule corona may comprise 1-5000 distinct glycoproteins/glycopeptides. A biomolecule corona may comprise 2-2000 distinct glycoproteins/glycopeptides. A biomolecule corona may comprise 3-1000 distinct glycoproteins/glycopeptides. A biomolecule corona may comprise 4-500 distinct glycoproteins/glycopeptides. A biomolecule corona may comprise 5-500 distinct glycoproteins/glycopeptides. A biomolecule corona may comprise 6-500 distinct glycoproteins/glycopeptides. A biomolecule corona may comprise 7-500 distinct glycoproteins/glycopeptides. A biomolecule corona may comprise 8-500 distinct glycoproteins/glycopeptides. A biomolecule corona may comprise 9-500 distinct glycoproteins/glycopeptides. A biomolecule corona may comprise 10-500 distinct glycoproteins/glycopeptides. A biomolecule corona may comprise 1-400 distinct glycoproteins/glycopeptides. A biomolecule corona may comprise 2-400 distinct glycoproteins/glycopeptides. A biomolecule corona may comprise 3-400 distinct glycoproteins/glycopeptides. A biomolecule corona may comprise 4-400 distinct glycoproteins/glycopeptides. A biomolecule corona may comprise 5-400 distinct glycoproteins/glycopeptides. A biomolecule corona may comprise 6-400 distinct glycoproteins/glycopeptides. A biomolecule corona may comprise 7-400 distinct glycoproteins/glycopeptides. A biomolecule corona may comprise 8-400 distinct glycoproteins/glycopeptides. A biomolecule corona may comprise 9-400 distinct glycoproteins/glycopeptides. A biomolecule corona may comprise 10-400 distinct glycoproteins/glycopeptides. A biomolecule corona may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, or 510 glycopeptides, or a range of glycopeptides defined by any two of the aforementioned integers. A biomolecule corona may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150, glycoproteins, or a range of glycoproteins defined by any two of the aforementioned integers.

A particle may include a metal. For example, a particle may include gold, silver, copper, nickel, cobalt, palladium, platinum, iridium, osmium, rhodium, ruthenium, rhenium, vanadium, chromium, manganese, niobium, molybdenum, tungsten, tantalum, iron, or cadmium, or a combination thereof.

A particle may be magnetic (e.g., ferromagnetic or ferrimagnetic). A particle comprising iron oxide may be magnetic. A particle may include a superparamagnetic iron oxide nanoparticle (SPION).

A particle may include a polymer. Examples of polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). A particle may be made from a combination of polymers.

A particle may include a lipid. Examples of lipids include dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, or cholesterol. A particle may be made from a combination of lipids.

Further examples of materials include silica, carbon, carboxylate, polyacrylic acid, carbohydrates, dextran, polystyrene, dimethylamine, amines, or silanes. Some examples of particles include a carboxylate SPION, a phenol-formaldehyde coated SPION, a silica-coated SPION, a polystyrene coated SPION, a carboxylated Poly(styrene-co-methacrylic acid), P(St-co-MAA) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(vinylbenzyltrimethylammonium chloride) (PVBTMAC) coated SPION, caboxylate coated with peracetic acid, a poly(oligo(ethylene glycol) methyl ether methacrylate) (POEGMA)-coated SPION, a polystyrene carboxyl functionalized particle, a carboxylic acid particle, a particle with an amino surface, a silica amino functionalized particle, a particle with a Jeffamine surface, or a silica silanol coated particle.

Some examples of nanoparticles include the following: P-033 (carboxylate microparticle, surfactant free), P-039 (polystyrene carboxyl functionalized), P-047 (silica), P-053 (amino surface microparticle, 0.4-0.6 μm), P-065 (silica), P-073 (dextran based coating, 0.13 μm), S-003 (silica-coated (SPION), S-006 (N-(3-trimethoxysilylpropyl)diethylenetriamine coated SPION), S-007 (poly(N-(3-(dimethylamino)propyl) methacrylamide) (PDMAPMA)-coated SPION), or S-010 (carboxylate, polyacrylic acid coated SPION).

Particles of various sizes may be used. The particles may include nanoparticles. Nanoparticles may be from about 10 nanometers (nm) to about 1000 nm in diameter. For example, the nanoparticles can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. A nanoparticle may be less than 1000 nm in diameter. Some examples include diameters of about 50 nm, about 130 nm, about 150 nm, 400-600 nm, or 100-390 nm.

The particles may include microparticles. A microparticle may be a particle that is from about 1 micrometer (μm) to about 1000 μm in diameter. For example, the microparticles can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. A microparticle may be less than 1000 μm in diameter. Some examples include diameters of 2.0-2.9 μm.

The particles may include physiochemically distinct sets of particles (for example, 2 or more sets of physiochemically particles where 1 set of particles is physiochemically distinct from another set of particles. Examples of physiochemical properties include charge (e.g., positive, negative, or neutral) or hydrophobicity (e.g., hydrophobic or hydrophilic). The particles may include 2, 3, 4, 5, 6, 7, 8, 9, 10, 11, 12, 13, 14, 15, 16, 17, 18, 19, 20, or more sets of particles, or a range of sets of particles including any of said numbers of sets of particles.

A sample may be contacted with particles and internal standard biomolecules. The combination of nanoparticles with internal standards may include a combination of the internal standards and sample with one nanoparticle at a time, or with multiple nanoparticles in the same sample.

Samples may be contacted with at least 1, 2, 3, 4, 5, 6, 7, 8, 9, 10, 15, 20, 50, 100, 200, 500, 1000 or more particles. In some cases, samples may be contacted with at most 1, 2, 3, 4, 5, 6, 7, 8, 9, 10, 15, 20, 50, 100, 200, 500, 1000 or more particles. Samples may be contacted with 1 particle. Samples may be contacted with 2 particles. Samples may be contacted with 3 particles. Samples may be contacted with 4 particles. Samples may be contacted with 5 particles. Samples may be contacted with 6 particles. Samples may be contacted with 7 particles. Samples may be contacted with 8 particles. Samples may be contacted with 9 particles. Samples may be contacted with 10 particles. Samples may be contacted with more than 10 particles. The particle may be the same. The particle may be different. Different particles may have physiochemically distinct particles descried herein. Different particles may have various sizes, materials, or structure described herein.

Particles may be contacted with a sample or a solution comprising a protein/peptide or internal standard described herein (such as an internal standard protein or an internal standard peptide) for at least about 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 1 month or more, to generate a biomolecule corona. In some cases, particles may be contacted with a sample or a solution comprising a protein/peptide or standard protein/peptide for at most about 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, or 1 month, to generate a biomolecule corona. Particles may be contacted with a sample or a solution comprising a protein/peptide or internal standard at at least about −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 50° C., 60° C. or more. In some cases, particles may be contacted with a sample or a solution comprising a protein/peptide or internal standard at at most about −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 50° C., or 60° C.

Samples may be contacted with particles, for example prior to generating data. The data described herein may generated using particles. For example, a method may include contacting a sample with particles such that the particles adsorb biomolecules. The particles may attract different sets of biomolecules than would normally be measured accurately by performing an omics measurement directly on a sample. For example, a dominant biomolecule may make up a large percentage of certain type of biomolecules (e.g., proteins, transcripts, genetic material, or metabolites) in a sample. By adhering biomolecules to particles prior to analyzing them, a subset of biomolecules may be obtained that does not include the dominant biomolecule. Removing dominant biomolecules in this way may increase the accuracy of biomolecule measurements and sensitivity of an analysis using those measurements.

Using the particle described herein may facilitate isolating a particular or subset protein/peptide from a sample using the methods described herein, relative to those without using the particles (e.g., the particular or subset protein/peptide are enriched by the particle). The particles may have physiochemical properties that allow the particular or subset protein/peptide to be adsorbed onto the surface of the particle to generate the biomolecule corona. Subsequent isolation of the biomolecule corona and release of the adsorbed biomolecules from the particle may allow the biomolecules to be assayed or measure using the methods described herein. The biomolecule corona may comprise the particle described herein, and the biomolecules described herein. The biomolecule corona may comprise the particle described herein and a protein or peptide. The protein or peptide adsorbed onto the particle in a biomolecule corona may comprise a glycoprotein or glycopeptide.

A glycoprotein or glycopeptide may comprise a protein or peptide that is glycosylated. Glycosylation of a protein or peptide may comprise attaching or coupling a sugar moiety (saccharide moiety) to the protein or peptide (forming the glycoprotein or glycopeptide). The sugar or saccharide moiety may comprise any of those sugars or saccharides described herein. Glycosylation is a prevalent post-translational modification comprising about ˜50% of the human proteins are glycosylated. Glycoproteins or glycopeptides can be involved in various biological processes (protein folding, cell growth, cell adhesion, immune function, disease condition comprising cancer, or a combination thereof).

Glycosylation may comprise N-linked glycosylation, O-linked glycosylation, C-linked glycosylation, S-linked glycosylation, glycation, or a combination thereof.

N-linked glycans are characterized by their five-saccharide moiety, composed of two N-Acetylglucosamine (GlcNAc) followed by three mannose (Man) units resulting in two available antennae for further glycosylation. N-linked glycosylation may comprise the attachment of saccharide moieties (such as oligosaccharides) to a nitrogen atom (such as N4 of asparagine residues) of the protein/peptide. N-linked glycosylation may comprise the covalent attachment of the amide group on an asparagine (N) amino acid to a glycoform. The sequon of the peptide backbone may comprise the motif of NXS/T, where X is not a proline. N-linked glycans are characterized by their five saccharide moieties, composed of two N-Acetylglucosamine (GlcNAc) followed by three mannose (Man) units resulting in two available antennae for further glycosylation. N-glycosylation can occur on secreted, or membrane bound proteins/peptides. In eukaryotic cells, N-glycosylation can be initiated as a co-translational event in the endoplasmic reticulum, wherein preassembled blocks of 14 sugar moieties (such as 2 N-acetylglucosamines, 9 mannoses and 3 glucoses) are first added to the nascent peptide. After cleavage of 3 glucose and 1 mannose residues, the protein can then be transferred to the Golgi apparatus where the glycans lose a variable number of mannose residues and acquire a more complex structure (the process is referred to as terminal glycosylation). Mature N-glycans may comprise 3 types: high mannose (those that have escaped terminal glycosylation), hybrid complex (with different combinations of mannose, N-acetylglucosamine, N-acetylgalactosamine, fucose and sialic acid residues). The consensus sequence for N-glycosylation can comprise Asn-Xaa-Ser/Thr (where Xaa is not Pro; note that Thr is more common than Ser) or Asn-X-Cys.

O-linked glycans are the attachment of the hydroxyl functional group on serine (S) or threonine (T) amino acids to a glycoform with no specific sequence motif O-linked glycans may comprise the attachment of the hydroxyl functional group on serine (S) or threonine (T) amino acids to a glycoform with no specific sequence motif O-linked glycans may comprise various different core structures. Core 1 structure, usually more commonly found, may comprise Galβ1-3GalNAc. Other less commonly found structures may comprise: Core 2 may comprise GlcNAcβ1-6(Galβ1-3)GalNAcα, Core 3 is GlcNAcβ1-3GalNAcα; Core 4 may comprise GlcNAcβ1-6(GlcNAcβ1-3)GalNAcα; Core 5 may comprise GalNAcα1-3GalNAcα; Core 6 may comprise GlcNAcβ1-6GalNAcα′ Core 7 may comprise GalNAcα1-6GalNAcα; Core 8 may comprise Galα1-3GalNAcα.3 Additionally saccharides may attach to any of these cores. O-linked glycosylation of secreted and membrane bound proteins may comprise a post-translational event that takes place in the cis-Golgi compartment after N-glycosylation and folding of the protein. O-linked glycosylation can comprise the attachment of glycans to serine and threonine, or to hydroxyproline and hydroxylysine. O-linked glycans can be involved in protein localization and trafficking, protein solubility, antigenicity and cell-cell interactions. O-linked glycans can eb built up in a stepwise fashion with sugars added incrementally. O-glycosylation in secreted and membrane-bound mammalian proteins/peptides can comprise the addition of reducing terminal N-acetylgalactosamine (GalNAc, a mucin-type glycan). The reducing terminal GalNAc residue can be further extended with galactose (Gal), N-acetylglucosamine (GlcNAc) or GlcNAc and Gal resulting in 8 common core structures, which can be decorated with the addition of up to three sialic acid residues. Some cytoplasmic and nuclear proteins/peptides can comprise a simple O-linked glycans in which a single N-acetylglucosamine residue is linked to a serine or a threonine. This modification may be found in plants and filamentous fungi. This type of O-linked glycosylation can be involved in the modulation of the biological activity of intracellular proteins/peptides. In some cases, the same residue may be subject to competing phosphorylation and O-linked glycosylation.

C-linked glycosylation can comprise the covalent attachment of a mannose residue to a tryptophan residue within an extracellular protein. Two recognition signals for C-mannosylation may comprise: W-X-X-W (in which the first or both tryptophan residues become mannosylated) or W-S/T-X-C.

S-linked glycosylation can comprise the attachment of oligosaccharides to the sulfur atom of the cysteine.

Glycation may comprise the non-enzymatic attachment of reducing sugars to the nitrogen atoms of proteins/peptides (both to the N-terminus and to lysine and histidine side chains). Glycation may comprise the Maillard reaction. In some cases, the sugar moieties bound to glycated proteins/peptides are gradually modified to become Advanced Glycation Endproducts (AGEs), Proteins or peptides with glycation may be associated with in a variety of disease conditions, such as type II diabetes mellitus, cancer, atherosclerosis, Alzheimer disease, and/or Parkinson disease.

Glycosylation may allow a biomolecule (such as a protein or peptide) to acquire a physiochemical property not found in the biomolecule not glycosylated. For example, various sugar moieties can have various physiochemical properties. When a protein or peptide is glycosylated, the protein or peptide can then acquire physiochemical properties of the sugar moieties, wherein the protein or peptide not glycosylated may not have the same physiochemical properties. Additionally, various distinct proteins or peptides, when glycosylated, can acquire the physiochemical properties of the sugar moieties. Hence this set of glycosylated distinct proteins or peptides can share similar physiochemical properties. By using particles with at least matching physiochemical properties, the particles can enrich this set of glycosylated distinct proteins or peptides. Thus, a beneficial advantage of the particles or methods described herein comprises using physiochemically distinct particles to enrich various set of glycosylated proteins or peptides. Additionally, in some cases, various endogenous biomolecules may have similar physiochemical properties (such as via similar chemical make-up, amino acid residues, post-translational modifications, folding properties, or a combination thereof), the physiochemically distinct particles described herein can enrich various sets of endogenous biomolecules with similar physiochemical properties. Various post-translational modifications can comprise glycosylation, ubiquitination, sumolyation, methylation, nitrosylation, methylation, acetylation, lipidation, or a combination thereof.

A method to enrich distinct proteins or peptides (or set thereof) (e.g., with a same or similar physiochemical property) using a particle described herein may isolate (or allow detection by methods described herein) at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, or 5000 more of the distinct proteins or peptides (or set thereof), relative to those isolated by methods that do not use the particles. A method to enrich distinct proteins or peptides (or set thereof) (e.g., with a same or similar physiochemical property) using a particle described herein may isolate (or allow detection by methods described herein) at least about 1%, 2%, 3%, 4%, 5%, 6%, 7%, 8%, 9%, 10%, 20%, 30%, 40%, 50%, 60%, 70%, 80%, 90%, 1-fold, 2-fold, 3-fold, 4-fold, 5-fold, 6-fold, 7-fold, 8-fold, 9-fold, 10-fold, 100-fold, 1000-fold, or 10000-fold more of the distinct proteins or peptides (or set thereof), relative to those isolated (or detected) by methods that do not use the particles, wherein the percentage or fold change is calculated by dividing [the numbers of the distinct proteins or peptides (or set thereof) isolated (or detected) by the methods using the particles] by the [the numbers of the distinct proteins or peptides (or set thereof) isolated (or detected) by the methods not using the particles]. A set of distinct proteins or peptides may comprise the collection of proteins or peptides with a same or similar physiochemical property(ies) or with similar modification (such a post-translational modification including but not limited to glycosylation).

A method to enrich distinct proteins or peptides (or set thereof) with more than one physiologically distinct particles described herein may isolate (or allow detection by methods described herein) at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, or 5000 more of the distinct proteins or peptides (or set thereof), relative to those isolated (or detected) by methods that use only one physiologically distinct particle. The distinct proteins or peptides may include distinct glycoproteins or glycopeptides. A method to enrich distinct proteins or peptides (or set thereof) with more than one physiochemical distinct particles described herein may isolate (or allow detection by methods described herein) at least about 1%, 2%, 3%, 4%, 5%, 6%, 7%, 8%, 9%, 10%, 20%, 30%, 40%, 50%, 60%, 70%, 80%, 90%, 1-fold, 2-fold, 3-fold, 4-fold, 5-fold, 6-fold, 7-fold, 8-fold, 9-fold, 10-fold, 100-fold, 1000-fold, or 10000-fold more of the distinct proteins or peptides (or set thereof), relative to those isolated (or detected) by methods that do not use the particles, wherein the percentage or fold change is calculated by dividing [the numbers of the distinct proteins or peptides (or set thereof) isolated by the methods using more than one physiochemically distinct particles] by the [the numbers of the distinct proteins or peptides (or set thereof) isolated by the methods using only one particle].

Disclosed herein are methods that include contacting a biofluid sample of a subject with particles to form biomolecule coronas comprising a number of distinct glycoproteins or glycopeptides adsorbed to the particles. Disclosed herein are methods that include obtaining measurements of a number of distinct glycoproteins or glycopeptides. Disclosed herein are methods that include contacting a biofluid sample of a subject with particles to form biomolecule coronas comprising a number of distinct glycoproteins or glycopeptides adsorbed to the particles; and obtaining measurements of the number distinct glycoproteins or glycopeptides. The number of distinct glycoproteins or glycopeptides may include about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 125, about 150, about 175, about 200, about 225, about 250, about 275, about 300, about 325, about 350, about 375, about 400, about 425, about 450, about 475, or about 500, or a range defined by any 2 of the aforementioned numbers. The number of distinct glycoproteins or glycopeptides may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, or at least 500. The number of distinct glycoproteins or glycopeptides may include less than 2, less than 3, less than 4, less than 5, less than 6, less than 7, less than 8, less than 9, less than 10, less than 11, less than 12, less than 13, less than 14, less than 15, less than 16, less than 17, less than 18, less than 19, less than 20, less than 25, less than 30, less than 35, less than 40, less than 45, less than 50, less than 55, less than 60, less than 65, less than 70, less than 75, less than 80, less than 85, less than 90, less than 95, less than 100, less than 125, less than 150, less than 175, less than 200, less than 225, less than 250, less than 275, less than 300, less than 325, less than 350, less than 375, less than 400, less than 425, less than 450, less than 475, or less than 500.

d. Internal Standards and Uses Thereof

The methods provided herein may comprise use of an internal standard. Internal standards may comprise biomolecules having predetermined identities or quantities. The internal standard may comprise a protein or peptide of a predetermined identity or quantity. The internal standard may comprise a post-translationally modified protein or peptides having a predetermined identity or quantity. The internal standard may comprise a glycoprotein having a predetermined identity and quantity. The internal standard may comprise a glycopeptide having a predetermined identity and quantity. Internal standards may comprise glycoproteins or glycopeptides of predetermined identities or quantities. Internal standards may comprise glycan moieties of predetermined identities or quantities. The internal standard may be referred to as a reference biomolecule.

The internal standard may allow quantification of the biomolecules in the biomolecule corona. In some cases, the internal standard may be added into the sample prior to the sample being contacted with a particle. In some cases, the internal standard may be added into the sample during the sample is being contacted with a particle. In some cases, the internal standard may be added into the sample subsequent to the sample being contacted with a particle. In some cases, the internal standard may contact a particle prior to the contacting between the biomolecules and the particle. In some cases, the internal standard may contact a particle during the contacting between the biomolecules and the particle. In some cases, the internal standard may contact a particle subsequent to the contacting between the biomolecules and the particle. In some cases, the internal standard may be added to the biomolecules subsequent to the biomolecules being released from the biomolecule corona.

The internal standard and the biomolecule may comprise the same class of biomolecules. For example, the internal standard of a protein or peptide comprises a protein or peptide. The internal standard of a glycoprotein or glycopeptide comprises a glycoprotein or a glycopeptide. The internal standard of a protein or peptide may comprise a glycoprotein or a glycopeptide. In some cases, an internal standard of a glycoprotein/glycopeptide may comprise proteins/peptides not glycosylated. In some cases, the internal standard of a glycoprotein/glycopeptide may comprise proteins/peptides that are glycosylated and proteins/peptides not glycosylated. The internal standard of a glycan moiety may comprise the glycan moiety. An internal standard may include a biomolecule that is added in a constant or pre-determined amount to the biological sample.

An internal standard may be labeled. An internal standard may not be labeled in some cases. The reference biomolecule may be unlabeled but with known property. For example, the reference biomolecule can be a plurality of polypeptides with known molar ratio and mass, which can yield reference measurements (e.g., functioning as internal standards in mass spectrometry measurements). Internal standards may comprise a non-endogenous labeled version of the endogenous biomolecules. The molecules used as internal standard may be reference molecules or reference biomolecules.

The reference biomolecule may be added to the biological sample for generating the measurements described herein. The method may include combining the first or second sample with the reference biomolecules, measuring the reference biomolecules with the biomolecules, and using the reference biomolecules to obtain the second measurements. The reference biomolecule may be detected by mass spectrometry or another method for measuring biomolecules described herein. In some aspects, the reference biomolecule is added to the biological sample before or after the biological sample is contacted with a particle or particles.

Using the predetermined identities and quantities of the internal standard, the methods disclosed herein can determine the identity quantity of the biomolecules (such as proteins/peptides or the glycosylated thereof or glycan moieties released from thereof) in the sample, adsorbed onto the particle, of the biomolecule corona, or a combination thereof. Such a determination may comprise generating a standard curve using the internal standards and quantifying the biomolecules using the standard curve. Generating protein or peptide data using known amounts of labeled internal reference proteins or peptides may be referred to as “PiQuant.” The internal reference proteins/peptides (or glycosylated thereof) may be spiked into a sample or a solution with a sample, particles, biomolecule coronas, or biomolecules released from the particle/biomolecule corona. The internal reference proteins/peptides may be used to identify mass spectra of individual endogenous biomolecules (protein, peptides, the glycosylated thereof, or a combination thereof). The internal reference proteins/peptides may be used as standards for determining amounts of the individual endogenous biomolecules. Proteomic measurements may be generated based on amounts of proteins/peptides added into a solution of a sample, particles, biomolecule coronas, or biomolecules released from the particle/biomolecule corona. Proteomic measurements may be generated based on amounts of labeled proteins added into a sample of the one or more biofluid samples. The internal standard biomolecules (such as proteins/peptides or glycoproteins or glycopeptides) may be not labeled.

A method may include obtaining measurements, for example measurements of glycoproteins or glycopeptides, or measurements of glycans or other carbohydrates. A method may include obtaining measurements such as measurements of glycoproteins or glycopeptides A method may include obtaining a measurement such as a glycoprotein or glycopeptide measurement. Obtaining the measurements may include combining glycoproteins or glycopeptides with labeled or unlabeled glycoproteins or glycopeptides, or with labeled or unlabeled non-glycosylated forms of the glycoproteins or glycopeptides. Obtaining the measurements may include combining glycoproteins or glycopeptides with labeled or unlabeled glycoproteins or glycopeptides. Obtaining a measurement may include combining a glycoprotein with a labeled version of the glycoprotein. Obtaining a measurement may include combining a glycoprotein with an unlabeled version of the glycoprotein. Obtaining a measurement may include combining a glycopeptide with a labeled version of the glycopeptide. Obtaining a measurement may include combining a glycopeptide with an unlabeled version of the glycopeptide. Obtaining the measurements may include combining glycoproteins or glycopeptides with labeled or unlabeled non-glycosylated forms of the glycoproteins or glycopeptides. When a non-glycosylated form of a glycoprotein or glycopeptide is used as an internal standard, measurements may be made of both glycosylated and non-glycosylated forms of an endogenous glycoprotein or glycopeptide. When measurements are obtained of both glycosylated and non-glycosylated forms of an endogenous glycoprotein or glycopeptide, a ratio such as a ratio of glycosylated to non-glycosylated glycoproteins or a ratio of glycosylated to non-glycosylated glycopeptides may be obtained. The ratio may be used as a biomarker in analyzing a biological state. Any number of ratios of various glycoproteins or glycopeptides to their respective non-glycosylated versions may be obtained and used herein. Obtaining a measurement may include combining a glycoprotein with a labeled non-glycosylated form of the glycoprotein. Obtaining a measurement may include combining a glycoprotein with an unlabeled non-glycosylated form of the glycoprotein. Obtaining a measurement may include combining a glycopeptide with a labeled non-glycosylated form of the glycopeptide. Obtaining a measurement may include combining a glycopeptide with an unlabeled non-glycosylated form of the glycopeptide.

Some aspects relate to a ratio of a glycoprotein or glycopeptide that is glycosylated. A method may include calculating a ratio of glycosylated glycoprotein or glycopeptide over a total amount of glycosylated and nonglycosylated glycoprotein or glycopeptide. In some aspects, a glycosylation site is not necessary glycosylated. A ratio glycosylation may be useful an indicator for a disease status or other biological state.

Some methods include contacting a sample (e.g., biofluid sample) from a subject with particles to form a biomolecule corona comprising glycoproteins adsorbed to the particles. Some methods include combining glycoproteins or glycopeptides with an internal standard. The glycoproteins or glycopeptides may be endogenous to the sample. The internal standard may be exogenous to the sample. Some methods include contacting a biofluid sample from a subject with particles to form a biomolecule corona comprising glycoproteins adsorbed to the particles; and combining the glycoproteins or glycopeptides with an internal standard. The internal standard may include a labeled glycoprotein. The internal standard may include a labeled glycopeptide. The internal standard may include a labeled non-glycosylated form of a glycoprotein. The internal standard may include a labeled non-glycosylated form of a glycopeptide. The internal standard may include a non-labeled glycoprotein. The internal standard may include a non-labeled glycopeptide. The internal standard may include a non-labeled non-glycosylated form of a glycoprotein. The internal standard may include a non-labeled non-glycosylated form of a glycopeptide. Any number of internal standards may be used (e.g., for different glycoproteins or glycopeptides).

Some methods include contacting a sample (e.g. biofluid sample) from a subject with particles to form a biomolecule corona comprising glycoproteins adsorbed to the particles; and combining the glycoproteins or glycopeptides with labeled glycoproteins or glycopeptides, or with labeled or unlabeled non-glycosylated forms of the glycoproteins or glycopeptides. Some methods include contacting a biofluid sample from a subject with particles to form a biomolecule corona comprising glycoproteins adsorbed to the particles. Some methods include combining glycoproteins or glycopeptides with labeled glycoproteins or glycopeptides. Some methods include combining glycoproteins or glycopeptides with labeled or unlabeled non-glycosylated forms of the glycoproteins or glycopeptides.

Some methods include contacting a sample from a subject with particles to form biomolecule coronas comprising glycoproteins or glycopeptides adsorbed to the particles. The method may include combining the glycoproteins or glycopeptides of the biomolecule coronas with labeled glycoproteins or glycopeptides, or with labeled or unlabeled non-glycosylated forms of the glycoproteins or glycopeptides. The method may also include separating the glycoproteins or glycopeptides and other biomolecules of the biomolecule coronas from the particles. The method may also include separating the glycoproteins or glycopeptides and other biomolecules of the biomolecule coronas from the particles. The method may also include enriching the glycoproteins or glycopeptides relative to the other biomolecules of the biomolecule coronas. Some methods include contacting a sample from a subject with particles to form biomolecule coronas comprising glycoproteins or glycopeptides adsorbed to the particles; separating the glycoproteins or glycopeptides and other biomolecules of the biomolecule coronas from the particles; enriching the glycoproteins or glycopeptides relative to the other biomolecules of the biomolecule coronas; and combining the glycoproteins or glycopeptides of the biomolecule coronas with labeled glycoproteins or glycopeptides, or with labeled or unlabeled non-glycosylated forms of the glycoproteins or glycopeptides.

Some aspects include combining glycans with internal standard glycans such as labeled glycans. For example, the endogenous glycans may be separated from glycoproteins or glycopeptides endogenous to a sample. Some aspects include combining an endogenous glycan with an internal standard glycan such as labeled glycan. Some aspects include combining glycan moieties separated from a glycoprotein or glycopeptide with a labeled glycan. In some aspects, the glycan moiety and the labeled glycan moiety are a same glycan moiety. In some aspects, the glycan moiety and the labeled glycan moiety are different glycan moieties. Some aspects include measuring an amount of the glycan moiety or the labeled glycan moiety. Some aspects include measuring an amount of the glycan moiety or the labeled glycan moiety by mass spectroscopy. In some aspects, a step is conducted in the presence of heavy water comprising an isotope. In some aspects, the heavy water comprises deuterium. Some aspects include introducing the isotope to a glycosylation site of the glycoproteins or glycopeptides that is de-glycosylated subsequent to a release of the glycan moiety from the glycoproteins or glycopeptides. Some aspects include measuring an amount of a de-glycosylated glycoprotein or glycopeptide labeled by the isotope and an amount of glycoproteins or glycopeptides that are not labeled. Some aspects include calculating a ratio of the amount of a de-glycosylated glycoprotein or glycopeptide labeled by the isotope and the amount of glycoproteins or glycopeptides that are not labeled. In some aspects, the ratio may comprise the amount of a de-glycosylated glycoprotein or glycopeptide labeled by the isotope divided by a total amount comprising the amount of a de-glycosylated glycoprotein or glycopeptide labeled by the isotope and the amount of glycoproteins or glycopeptides that are not labeled.

The internal standard may comprise at least about 1 femtomolar (fM), 10 fM, 100 fM, 1 picomolar (pM), 10 pM, 100 pM, 1 nanomolar (nM), 10 nM, 100 nM, 1 micromolar (μM), 10 μM, 100 μM, 1 millimolar (mM), 10 mM, 100 mM, 1 molar (M), 10 M, 100 M, 1000 M, 10000 M, 100000 M or more reference biomolecules. In some cases, the internal standard may comprise at most about 1 femtomolar (fM), 10 fM, 100 fM, 1 picomolar (pM), 10 pM, 100 pM, 1 nanomolar (nM), 10 nM, 100 nM, 1 micromolar (μM), 10 μM, 100 μM, 1 millimolar (mM), 10 mM, 100 mM, 1 molar (M), 10 M, 100 M, 1000 M, 10000 M, or 100000 M reference biomolecules. The internal standard may comprise at least 2, at least 3, at least 4, at least 5, at least 10, at least 50, at least 100, at least 250, at least 500, at least 750, at least 1000, at least 1500, at least 2000, at least 2500, at least 5000, at least 7500, at least 10,000, at least 15,000, at least 20,000, or at least 25,000 distinct reference biomolecules. In some instances, the reference biomolecules include less than 5, less than 10, less than 50, less than 100, less than 250, less than 500, less than 750, less than 1000, less than 1500, less than 2000, less than 2500, less than 5000, less than 7500, less than 10,000, less than 15,000, less than 20,000, or less than 25,000 distinct reference biomolecules. The internal standard may comprise at least about 1 femtomolar (fM), 10 fM, 100 fM, 1 picomolar (pM), 10 pM, 100 pM, 1 nanomolar (nM), 10 nM, 100 nM, 1 micromolar (μM), 10 μM, 100 μM, 1 millimolar (mM), 10 mM, 100 mM, 1 molar (M), 10 M, 100 M, 1000 M, 10000 M, 100000 M or more proteins/peptides (or glycosylated version thereof) of the internal standard. In some cases, the internal standard may comprise at most about 1 femtomolar (fM), 10 fM, 100 fM, 1 picomolar (pM), 10 pM, 100 pM, 1 nanomolar (nM), 10 nM, 100 nM, 1 micromolar (μM), 10 μM, 100 μM, 1 millimolar (mM), 10 mM, 100 mM, 1 molar (M), 10 M, 100 M, 1000 M, 10000 M, or 100000 M proteins/peptides (or glycosylated version thereof) of the internal standard. The internal standard may comprise at least 2, at least 3, at least 4, at least 5, at least 10, at least 50, at least 100, at least 250, at least 500, at least 750, at least 1000, at least 1500, at least 2000, at least 2500, at least 5000, at least 7500, at least 10,000, at least 15,000, at least 20,000, or at least 25,000 distinct proteins/peptides (or glycosylated version thereof). In some instances, the reference biomolecules include less than 5, less than 10, less than 50, less than 100, less than 250, less than 500, less than 750, less than 1000, less than 1500, less than 2000, less than 2500, less than 5000, less than 7500, less than 10,000, less than 15,000, less than 20,000, or less than 25,000 distinct proteins/peptides (or glycosylated version thereof).

A reference biomolecule of the internal standard may have a molecular size of at least about 1×10\hat{ }−15 angstroms (Å), 1×10\hat{ }−14 Å, 1×10\hat{ }−13 Å, 1×10\hat{ }−12 Å, 1×10\hat{ }−11 Å, 1×10\hat{ }−10 Å, 1×10\hat{ }−9 Å, 1×10\hat{ }−8 Å, 1×10\hat{ }−7 Å, 1×10\hat{ }−6 Å, 1×10\hat{ }−5 Å, 1×10\hat{ }−4 Å, 1×10\hat{ }−3 Å, 1×10\hat{ }−2 Å, 1×10\hat{ }−1 Å, 1×10\hat{ }0 Å, 1×10\hat{ }1 Å, 1×10\hat{ }2 Å, 1×10\hat{ }3 Å, 1×10\hat{ }4 Å, 1×10\hat{ }5 Å, 1×10\hat{ }6 Å, 1×10\hat{ }7 Å, 1×10\hat{ }8 Å, 1×10\hat{ }9 Å, 1×10\hat{ }10 Å, 1×10\hat{ }11 Å, 1×10\hat{ }12 Å, 1×10\hat{ }13 Å, 1×10\hat{ }14 Å, or 1×10\hat{ }15 Å. In some cases, a reference biomolecule of the internal standard may have a molecular size of at most about 1×10\hat{ }−15 Å, 1×10\hat{ }−14 Å, 1×10\hat{ }−13 Å, 1×10\hat{ }−12 Å, 1×10\hat{ }−11 Å, 1×10\hat{ }−10 Å, 1×10\hat{ }−9 Å, 1×10\hat{ }−8 Å, 1×10\hat{ }−7 Å, 1×10\hat{ }−6 Å, 1×10\hat{ }−5 Å, 1×10\hat{ }−4 Å, 1×10\hat{ }−3 Å, 1×10\hat{ }−2 Å, 1×10\hat{ }−1 Å, 1×10\hat{ }0 Å, 1×10\hat{ }1 Å, 1×10\hat{ }2 Å, 1×10\hat{ }4 Å, 1×10\hat{ }4 Å, 1×10\hat{ }5 Å, 1×10\hat{ }6 Å, 1×10\hat{ }7 Å, 1×10\hat{ }8 Å, 1×10\hat{ }9 A, 1×10\hat{ }10 Å, 1×10\hat{ }11 Å, 1×10\hat{ }12 Å, 1×10\hat{ }13 Å, 1×10\hat{ }14 Å, or 1×10\hat{ }15 Å.

A reference biomolecule of the internal standard may have a molecular mass of at least about 1×10\hat{ }−15 daltons, 1×10\hat{ }−14 daltons, 1×10\hat{ }−13 daltons, 1×10\hat{ }−12 daltons, 1×10\hat{ }−11 daltons, 1×10\hat{ }−10 daltons, 1×10\hat{ }−9 daltons, 1×10\hat{ }−8 daltons, 1×10\hat{ }−7 daltons, 1×10\hat{ }−6 daltons, 1×10\hat{ }−5 daltons, 1×10\hat{ }−4 daltons, 1×10\hat{ }−3 daltons, 1×10\hat{ }−2 daltons, 1×10\hat{ }−1 daltons, 1×10\hat{ }0 daltons, 1×10\hat{ }1 daltons, 1×10\hat{ }2 daltons, 1×10\hat{ }3 daltons, 1×10 4 daltons, 1×10 5 daltons, 1×10\hat{ }6 daltons, 1×10\hat{ }7 daltons, 1×10\hat{ }8 daltons, 1×10\hat{ }9 daltons, 1×10\hat{ }10 daltons, 1×10\hat{ }11 daltons, 1×10\hat{ }12 daltons, 1×10\hat{ }13 daltons, 1×10\hat{ }14 daltons, 1×10\hat{ }15 daltons. A reference biomolecule of the internal standard may have a molecular mass of at least about 1×10\hat{ }−15 daltons, 1×10\hat{ }−14 daltons, 1×10\hat{ }−13 daltons, 1×10\hat{ }−12 daltons, 1×10\hat{ }−11 daltons, 1×10\hat{ }−10 daltons, 1×10\hat{ }−9 daltons, 1×10\hat{ }−8 daltons, 1×10\hat{ }−7 daltons, 1×10\hat{ }−6 daltons, 1×10\hat{ }−5 daltons, 1×10\hat{ }−4 daltons, 1×10\hat{ }−3 daltons, 1×10\hat{ }−2 daltons, 1×10\hat{ }−1 daltons, 1×10\hat{ }0 daltons, 1×10\hat{ }1 daltons, 1×10\hat{ }2 daltons, 1×10\hat{ }3 daltons, 1×10\hat{ }4 daltons, 1×10\hat{ }5 daltons, 1×10\hat{ }6 daltons, 1×10\hat{ }7 daltons, 1×10\hat{ }8 daltons, 1×10\hat{ }9 daltons, 1×10\hat{ }10 daltons, 1×10\hat{ }11 daltons, 1×10\hat{ }12 daltons, 1×10\hat{ }13 daltons, 1×10\hat{ }14 daltons, or 1×10\hat{ }15 daltons.

Within the reference biomolecules, individual labeled biomolecules may correspond to the individual endogenous biomolecules. For example, a solution may comprise proteins/peptides, and the endogenous proteins to be determined may comprise 100-1500 different proteins and the labeled reference biomolecules may comprise the same 100-1500 proteins with predetermined quantities and each labeled biomolecule may comprise a label.

As an example, a sample comprises endogenous protein/peptide A, endogenous protein/peptide B, and endogenous protein/peptide C. Endogenous proteins/peptides A, B and C are difficult to measure because of their low abundance. Upon spiking predetermined amounts of isotopically labeled versions of proteins/peptides A, B and C (reference proteins/peptides) into the sample, endogenous proteins/peptides A, B, and C, and the isotopically labeled versions of proteins/peptides A, B and C are analyzed together using mass spectrometry. Because the isotopically labeled versions are heavier, their mass spectra are shifted, and are distinguishable from mass spectra for the endogenous proteins/peptides. The isotopically labeled versions are more readily identifiable on a mass spectrometry readout thereby facilitating the identification of mass spectra for endogenous proteins/peptides A, B and C on the mass spectrometry readout. Because a predetermined amount of isotopically labeled proteins/peptides A, B, and C was added to spiked into the sample, their quantity is known, and the mass spectra for isotopically labeled proteins/peptides A, B, and C can quantify the endogenous proteins/peptides A, B, and C from the mass spectrometry readout. The accurate measurements of the endogenous proteins/peptides A, B, and C may be obtained by comparing the relative intensities of the mass spectrometry readouts for endogenous proteins/peptides A, B, and C relative to the intensities of the mass spectrometry readouts for the known concentrations or amounts of isotopically labeled proteins/peptides A, B, and C.

A method may include obtaining a first data set comprising first measurements of biomolecules adsorbed to particles from a first biological sample of a subject; and obtaining a second data set comprising second measurements of the biomolecules of the first biological sample or of a second biological sample of the subject. The second measurements may include measurements of endogenous biomolecules normalized or adjusted based on measurements of labeled reference biomolecules combined with the first biological sample or combined with the second biological sample. The labeled reference biomolecules are the same as the endogenous biomolecules, but each comprise a label. A method may include applying a first classifier to assign a first label corresponding to a biological state to the first data set; applying a second classifier to assign a second label corresponding to the biological state to the second data set; and combining the first label and the second label to obtain a combined label corresponding to the biological state.

A method may include obtaining measurements of endogenous biomolecules adsorbed to particles (e.g., nanoparticles) from a biological sample of a subject, and obtaining measurements of labeled reference biomolecules combined with the biological sample or combined with the endogenous biomolecules adsorbed to the particles. The labeled reference biomolecules may be the same as the endogenous biomolecules but also comprise a label. A method may include normalizing or adjusting the measurements of the endogenous biomolecules based on the measurements of the labeled reference biomolecules. A method may include applying a classifier to the normalized or adjusted measurements to assign a label corresponding to a biological state to the normalized or adjusted measurements.

A method may include contacting a biological sample of a subject with particles, thereby adsorbing endogenous biomolecules of the biological sample to the particles. A method may include combining the biological sample or the adsorbed endogenous biomolecules with internal standards of the biomolecules (which may comprise a label). A method may include combining the biological sample with internal standards of the biomolecules (which may comprise a label). A method may include combining the adsorbed endogenous biomolecules with internal standards of the biomolecules comprising a label. A method may include measuring the endogenous biomolecules and the internal standards to obtain endogenous biomolecule measurements and internal standard measurements.

The peptide or protein internal standards may be used in real-time control of a mass spectrometer based on measurement quality assessed as described herein to perform an adjustment, pause or stop data collection, rescheduling of sample or data collection, or provide automated notifications. For example, the peptide or protein internal standards may be used in real-time to adjust of internal voltages to provide a change in sensitivity (e.g., detector gain). The peptide or protein internal standards may be used in real-time to adjust a sample volume used for analysis of individual subjects. The peptide or protein internal standards may be used in real-time to adjust technical conditions to provide superior data quality. An example is real time evaluation of MS/MS spectra to determine if additional or reduced fragmentation energy is needed to create a MS/MS spectra above a defined threshold. The peptide or protein internal standards may be used in real-time to pause or stop data collection if instrument performance is below one, or several, defined performance thresholds. The peptide or protein internal standards may be used in real-time to reschedule individual samples or control samples to collect additional data either after instrument adjustments (e.g., voltages) or instrument maintenance (e.g., cleaning). Additional data collection may include additional quantitative data, biological data (e.g., collection of additional biologically relevant data based on detection of expected or unexpected biological changes via data driven control of a mass spectrometer), or technical data (e.g., adjustment of fragmentation energy). The peptide or protein internal standards may be used in real-time to automate a notification message sent directly to a user as a warning that a quality control (QC) performance threshold limit is approached or surpassed.

Real-time control of a mass spectrometer may include real-time control of mass spectrometry measurements. While being measured by the mass spectrometer, biomolecules in a sample may be mixed with internal standard reference biomolecules and may have been adsorbed or contacted with particles. The biomolecules measured using a mass spectrometer may include biomolecules adsorbed in a sample to a single type of particle or may include biomolecules adsorbed in a sample to multiple types of particles. The adsorption of biomolecules to multiple types of particles may include contact of the sample with multiple types of particles together or may include contact of aliquots of the sample separately with one or more particle types per aliquot and then the aliquots may be pooled for measuring the adsorbed biomolecules. The biomolecules in the sample may have contacted with particles and internal standard biomolecules. The combination of particles with internal standards may include a combination of the internal standards and sample with one particle at a time, or with multiple particles in the same sample. Some aspects may include multiple injections/sample/particle, and different decisions may be made in real-time during the measurement of each separate injection. Such an analysis may be repeated, and a decision process may be made across all particles. In some aspects, multiple particles are pooled together, and then a mass spectrometry analysis is performed.

The reference peptides or proteins of internal standards may be used in normalization of 2 or more samples through the use of either measured quantitative values of the reference peptides or proteins of the internal standards. Internal standards may be added to each sample either prior to after the processing by particles. Internal standards may be added to control samples (technical or biological) to provide known reference values. A variety of techniques (e.g., median or local regression such as LOESS) can be used to normalize differences in response as a function of processing by particles and/or measurement by mass spectrometry.

The reference peptides or proteins of internal standards may be used in establishing or determining the recovery of each protein processed utilizing particles. Determining the recovery of each protein may be useful for providing understanding of protein losses on a particle as a function of corona formation or PPI and available individual protein concentration after processing by particles. This information can be used to derive a far more accurate quantitation of endogenous biomolecules such as endogenous biomolecules adsorbed to particles.

The reference peptides or proteins of internal standards may be used in establishing or determining biological concentrations of proteins, and proteoforms, in individual patient samples. Internal standards added prior to processing of samples by particles may be useful for providing a measurement of the sample concentration of endogenous proteins or protoeforms.

The reference peptides or proteins of internal standards may be used in establishing or identifying sources of variability of processing samples by particles and mass spectrometry. Addition of internal standards after processing samples may provide a measurement of the technical variability associated with the measurement. Addition of internal standards prior to processing of samples may provide a direct measurement of technical variability for an entire sample processing process.

The reference peptides or proteins of internal standards may be used in collection of additional biologically relevant data (e.g., proteoforms) based the detection of expected or unexpected biological changes via data driven control of a mass spectrometer. Dependent on the data collected and analyzed in real time (e.g., MS/MS, Database search results, quantitation, or CCS value) a mass spectrometer may be controlled to generate additional data. When a protein is detected, or detected above a desired threshold, then the mass spectrometer can be directed to collect additional MS/MS data on predicted PTM or genetically modified version of the same peptide/protein.

Detection of discordant peptides may trigger additional data generation. The concentration of several unique peptides for a given protein may be either up or down regulated in the same direction relative to a reference concentration (e.g., a control sample concentration). When a discordant peptide is detected, then the instrument can be controlled in real time to collect data on the genetically modified version of the peptide (e.g., pre-calculated in a database). A discordant peptide may be due to either genetic modification (e.g., a mutation or single nucleotide polymorphism [SNP]) or apost-translational modification (PTM; e.g., glycosylation or phosphorylation). The additional data collected may be based on a database of predicted mass, retention times, CCS, Kendrick mass defect or predicted energy required to sequence the desired peptide (e.g., fragmentation modality and energy). The mode and energy of fragmentation may be determined based on the predicted modification one is attempting to detect (e.g., EAD/ETD for glycosylated proteins vs CID for SNP modified peptides).

The reference peptides or proteins of internal standards may be used in determination of one or multiple health status(s) through the quantitative peptide and protein measurements, comparison to known pattern of peptide and protein concentrations, and assessment.

The reference peptides or proteins of internal standards may be used in health status call based on the concentrations of multiple peptides/proteins in a single sample (e.g., CRC based on detected concentration of certain proteins (modified or unmodified). A database of signatures/classifiers may be used.

The reference biomolecule may be labeled. The label may include isotopic labeling or fluorescent labeling. The reference biomolecules may include an isotopic label, a mass tag, a barcode, a post-translation modification (PTM), or a biomolecule from a species different than a species of the subject in which the sample is extracted from. The reference biomolecules may include a label. The label may be isotopic. The reference biomolecules may include a mass tag. The reference biomolecules may include a barcode. The reference biomolecules may include a PTM. The reference biomolecules may include a biomolecule from a species different than a species of the subject in which the sample is extracted from. The reference biomolecules may include multiple labels such as isotopic labels, mass tags, barcodes, PTMs, or biomolecules from a species different than a species of the subject.

A reference biomolecule may include a molecule from a procaryotic cell or from a eucaryotic cell or a combination thereof. A reference biomolecule may be a molecule from a procaryotic cell. A reference biomolecule may be a molecule from a eucaryotic cell. Reference biomolecule(s) may be a molecule from a procaryotic cell and one from a eucaryotic cell. Reference biomolecule(s) may be a molecule from a virus, bacteria, archaea, protist, fungi, plant, invertebrate, vertebrate, or a combination thereof. Reference biomolecule(s) may be a molecule from a virus. Reference biomolecule(s) may be a molecule from a bacteria. Reference biomolecule(s) may be a molecule from a archaea. Reference biomolecule(s) may be a molecule from a protist. Reference biomolecule(s) may be a molecule from a fungi. Reference biomolecule(s) may be a molecule from a plant. Reference biomolecule(s) may be a molecule from a invertebrate. Reference biomolecule(s) may be a molecule from a vertebrate. A reference protein/peptide may be a protein/peptide from a procaryotic cell or from a eucaryotic cell or a combination thereof. A reference protein/peptide may be a protein/peptide from a procaryotic cell. A reference protein/peptide may be a protein/peptide from a eucaryotic cell. Reference protein/peptide(s) may be a protein/peptide from a procaryotic cell and one from a eucaryotic cell. Reference protein/peptide(s) may be a protein/peptide from a virus, bacteria, archaea, protist, fungi, plant, invertebrate, vertebrate, or a combination thereof. Reference protein/peptide(s) may be a protein/peptide from a virus. Reference protein/peptide(s) may be a protein/peptide from a bacteria. Reference protein/peptide(s) may be a protein/peptide from a archaea. Reference protein/peptide(s) may be a protein/peptide from a protist. Reference protein/peptide(s) may be a protein/peptide from a fungi. Reference protein/peptide(s) may be a protein/peptide from a plant. Reference protein/peptide(s) may be a protein/peptide from an invertebrate. Reference protein/peptide(s) may be a protein/peptide from a vertebrate. Reference protein/peptide(s) may comprise a mouse, bovine, hamster, chicken, rat or human protein/peptide. Reference protein/peptide(s) may comprise a human protein/peptide.

A label may comprise an isotope label. An isotope label may comprise an atom with a detectable variation in neutron count. Isotope label may be detected by the mass, vibrational mode, radioactive decay, or a combination thereof the isotope label. Mass spectrometry can detect the mass or a difference thereof of an isotope label using the mass of the isotope. Infrared spectroscopy can detect the vibrational modes or a difference thereof of an isotope label. Nuclear magnetic resonance can detect atoms with different gyromagnetic ratios. The radioactive decay can be detected through an ionization chamber or autoradiographs of gels. An isotope label may comprise a radioisotope label.

An isotope label may comprise deuterium (D or ²H), ¹²C, ¹³C, ¹⁴C, ¹⁵N, ¹⁷O, ¹⁸O, ¹H, or a combination thereof. In some cases, isotope label may comprise D. An isotope label may comprise ¹²C. An isotope label may comprise ¹³C. An isotope label may comprise ¹⁴C. An isotope label may comprise ¹⁵N. An isotope label may comprise ¹⁷O. An isotope label may comprise ¹⁸O. An isotope label may comprise ¹H. A ¹⁵N isotope label may comprise ¹⁵N₂ or ¹⁵N₄ A ¹³C isotope label may comprise ¹³C₃, ¹³C₅, ¹³C₆, ¹³C₉, or a combination thereof.

A reference biomolecule (such as a protein/peptide or glycosylated thereof) may at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more labels. In some cases, a reference biomolecule (such as a protein/peptide or glycosylated thereof) may at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 labels. A reference biomolecule (such as a protein/peptide or glycosylated thereof) may at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more isotope labels. In some cases, a reference biomolecule (such as a protein/peptide or glycosylated thereof) may at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 isotope labels.

In some instances, the biomolecule may comprise a glycan moiety release from a glycoprotein/glycopeptide. In some instances, the reference biomolecule may comprise a glycan moiety.

e. Data Generation

The methods disclosed herein may include obtaining data such as protein data or proteomic data, or using data generated from one or more samples collected from a subject. The data may include biomolecule measurements such as protein measurements. This section includes some ways of generating protein or proteomic data. Other types of proteomic data may also be generated. Descriptions of generating or analyzing proteomic data may be applied to methods of generating or analyzing individual biomolecules or sets of biomolecules that do not necessarily include proteomic data. The data may be labeled or identified as indicative of a disease or as not indicative of a disease.

The data described herein may include protein data. Protein data may include proteomic data. Proteomic data may involve data about proteins, peptides, or proteoforms. The protein data may include peptide data. The protein data may include protein group data. The protein data may include proteoform data. The protein data may include glycoprotein data. The glycoprotein data may include glycopeptide data. The glycoprotein data may include glycoprotein group data. In some embodiments, the proteomic data is generated from a method described herein. In some embodiments, the proteomic data is analyzed by a method described herein. This data may include just peptide or protein measurements (e.g., protein group measurements), or a combination of both. An example of a peptide is an amino acid chain. An example of a protein is a peptide or a combination of peptides. For example, a protein may include one, two or more peptides bound together. A protein may also include any post-translational modifications. Proteomic data may include data about various proteoforms. Proteoforms can include different forms of a protein produced from a genome with any variety of sequence variations, splice isoforms, or post-translational modifications (PTMs). An example of a post-translational modification includes glycosylation. Glycosylation may include N-glycosylation. The proteomic data may be generated using an unbiased, non-targeted approach, or may include a specific set of proteins. A protein may include a glycoprotein. A peptide may include a glycopeptide. Proteomic data may include glycoproteomic data.

Proteomic data may include information on the presence, absence, or amount of various proteins, peptides. For example, proteomic data may include amounts of proteins. A protein amount may be indicated as a concentration or quantity of proteins, for example a concentration of a protein in a biofluid. A protein amount may be relative to another protein or to another biomolecule. Proteomic data may include information on the presence of proteins or peptides. Proteomic data may include information on the absence of proteins or peptides. Proteomic data may be distinguished by subtype, where each subtype includes a different type of protein, peptide, or proteoform.

Proteomic data generally includes data on a number of proteins or peptides. For example, proteomic data may include information on the presence, absence, or amount of 1000 or more proteins or peptides. In some cases, proteomic data may include information on the presence, absence, or amount of 5000, 10,000, 20,000, or more peptides, proteins, or proteoforms. Proteomic data may even include up to about 1 million proteoforms. Proteomic data may include a range of proteins, peptides, or proteoforms defined by any of the aforementioned numbers of proteins, peptides, or proteoforms.

Proteomic data may be generated by any of a variety of methods. Generating proteomic data may include using a detection reagent that binds to a peptide or protein and yields a detectable signal. After use of a detection reagent that binds to a peptide or protein and yields a detectable signal, a readout may be obtained that is indicative of the presence, absence or amount of the protein or peptide. Generating proteomic data may include concentrating, filtering, or centrifuging a sample.

Proteomic data may be generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. Proteomic data may be generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, immunostaining, sequencing or a combination thereof. Some examples of methods for generating proteomic data include using mass spectrometry, a protein chip, or a reverse-phased protein microarray. Proteomic data may also be generated using immunoassays such as enzyme-linked immunosorbent assays, western blots, dot blots, or immunohistochemistry. Generating proteomic data may involve use of an immunoassay panel. Generating proteomic data may involve use of an O-link approach that includes sequencing for detection.

One way of obtaining proteomic data includes use of mass spectrometry. An example of a mass spectrometry method includes use of high resolution, two-dimensional electrophoresis to separate proteins from different samples in parallel, followed by selection or staining of differentially expressed proteins to be identified by mass spectrometry. Another method uses stable isotope tags to differentially label proteins from two different complex mixtures. The proteins within a complex mixture may be labeled isotopically and then digested to yield labeled peptides. Then the labeled mixtures may be combined, and the peptides may be separated by multidimensional liquid chromatography and analyzed by tandem mass spectrometry. A mass spectrometry method may include use of any chromatography described herein. A mass spectrometry method may include use of liquid chromatography-mass spectrometry (LC-MS), a technique that may combine physical separation capabilities of liquid chromatography (e.g., HPLC) with mass spectrometry. A mass spectrometry method may include use of HILIC. A mass spectrometry method may include use ERLIC. A mass spectrometry method may include use HILIC and ERLIC.

In addition to any of the above methods, generating proteomic data may include contacting a sample with particles such that the particles adsorb biomolecules comprising proteins. The adsorbed proteins may be part of a biomolecule corona. The adsorbed proteins may be measured or identified in generating the proteomic data.

For generating the protein or proteome data (proteomic data), a solution comprising at least about 1 picogram (pg), 2 pg, 5 pg, 10 pg, 20 pg, 50 pg, 100 pg, 200 pg, 500 pg, 1 nanogram (ng), 2 ng, 5 ng, 10 ng, 20 ng, 50 ng, 100 ng, 200 ng, 500 ng, 1 microgram (μg), 2 μg, 5 μg, 10 μg, 20 μg, 50 μg, 100 μg, 200 μg, 500 μg, 1 milligram (mg), 2 mg, 5 mg, 10 mg, 20 mg, 50 mg, 100 mg, 200 mg, 500 mg, 1 gram or more of proteins/peptides (or glycosylated thereof) may be applied to the mass spectrometer. For generating the protein or proteome data, a solution comprising at least about 1 picogram (pg), 2 pg, 5 pg, 10 pg, 20 pg, 50 pg, 100 pg, 200 pg, 500 pg, 1 nanogram (ng), 2 ng, 5 ng, 10 ng, 20 ng, 50 ng, 100 ng, 200 ng, 500 ng, 1 microgram (μg), 2 μg, 5 μg, 10 μg, 20 μg, 50 μg, 100 μg, 200 μg, 500 μg, 1 milligram (mg), 2 mg, 5 mg, 10 mg, 20 mg, 50 mg, 100 mg, 200 mg, 500 mg, or 1 gram of proteins/peptides (or glycosylated thereof) may be applied to the mass spectrometer. For generating the protein or proteome data, a solution comprising at least about 1×10\hat{ }−20 moles, 1×10\hat{ }−19 moles, 1×10\hat{ }−18 moles, 1×10\hat{ }−17 moles, 1×10\hat{ }−16 moles, 1×10\hat{ }−15 moles, 1×10\hat{ }−14 moles, 1×10\hat{ }−13 moles, 1×10\hat{ }−12 moles, 1×10\hat{ }−11 moles, 1×10\hat{ }−10 moles, 1×10\hat{ }−9 moles, 1×10\hat{ }−8 moles, 1×10\hat{ }−7 moles, 1×10\hat{ }−6 moles, 1×10\hat{ }−5 moles, 1×10\hat{ }−4 moles, 1×10\hat{ }−3 moles, 1×10\hat{ }−2 moles, 1×10\hat{ }−1 moles, 1×10\hat{ }0 moles, 1×10\hat{ }1 moles, 1×10\hat{ }2 moles, 1×10\hat{ }3 moles, 1×10\hat{ }4 moles, 1×10\hat{ }5 moles, 1×10\hat{ }6 moles, 1×10\hat{ }7 moles, 1×10\hat{ }8 moles, 1×10\hat{ }9 moles, 1×10 \hat{ }10 moles, 1×10\hat{ }11 moles, 1×10\hat{ }12 moles, 1×10\hat{ }13 moles, 1×10\hat{ }14 moles, 1×10\hat{ }15 moles, 1×10\hat{ }16 moles, 1×10\hat{ }17 moles, 1×10\hat{ }18 moles, 1×10\hat{ }19 moles, or 1×10\hat{ }20 moles of proteins/peptides (or glycosylated thereof) may be applied to the mass spectrometer. In some cases, for generating the protein or proteome data, a solution comprising at most about 1×10\hat{ }−20 moles, 1×10\hat{ }−19 moles, 1×10\hat{ }−18 moles, 1×10\hat{ }−17 moles, 1×10\hat{ }−16 moles, 1×10\hat{ }−15 moles, 1×10\hat{ }−14 moles, 1×10\hat{ }−13 moles, 1×10\hat{ }−12 moles, 1×10\hat{ }−11 moles, 1×10\hat{ }−10 moles, 1×10\hat{ }−9 moles, 1×10\hat{ }−8 moles, 1×10\hat{ }−7 moles, 1×10\hat{ }−6 moles, 1×10\hat{ }−5 moles, 1×10\hat{ }−4 moles, 1×10\hat{ }−3 moles, 1×10\hat{ }−2 moles, 1×10\hat{ }−1 moles, 1×10\hat{ }0 moles, 1×10\hat{ }1 moles, 1×10\hat{ }2 moles, 1×10\hat{ }3 moles, 1×10\hat{ }4 moles, 1×10\hat{ }5 moles, 1×10\hat{ }6 moles, 1×10\hat{ }7 moles, 1×10\hat{ }8 moles, 1×10\hat{ }9 moles, 1×10\hat{ }10 moles, 1×10\hat{ }11 moles, 1×10\hat{ }12 moles, 1×10\hat{ }13 moles, 1×10\hat{ }14 moles, 1×10\hat{ }15 moles, 1×10\hat{ }16 moles, 1×10\hat{ }17 moles, 1×10\hat{ }18 moles, 1×10\hat{ }19 moles, or 1×10\hat{ }20 moles of proteins/peptides (or glycosylated thereof) may be applied to the mass spectrometer. The solution may have been enriched, additionally enriched, or processed by the methods described herein.

A mass spectroscopy using a method described herein may detect at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, 5000 or more distinct proteins or peptides (e.g., including glycoproteins or glycopeptides). In some cases, the mass spectroscopy using a method described herein may detect at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, or 5000 distinct proteins or peptides (e.g., including glycoproteins or glycopeptides).

The mass spectroscopy using a method described herein may detect at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 1500, or 2000, distinct glycoproteins or glycopeptides. In some cases, the mass spectroscopy using a method described herein may detect at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 1500, or 2000, or 5000 distinct glycoproteins or glycopeptides. Mass spectroscopy using a method described herein may detect 1-5000 distinct proteins/peptides. Mass spectroscopy using a method described herein may detect 2-2000 distinct proteins/peptides. Mass spectroscopy using a method described herein may detect 3-1000 distinct proteins/peptides. Mass spectroscopy using a method described herein may detect 4-500 distinct proteins/peptides. Mass spectroscopy using a method described herein may detect 5-500 distinct proteins/peptides. Mass spectroscopy using a method described herein may detect 6-500 distinct proteins/peptides. Mass spectroscopy using a method described herein may detect 7-500 distinct proteins/peptides. Mass spectroscopy using a method described herein may detect 8-500 distinct proteins/peptides. Mass spectroscopy using a method described herein may detect 9-500 distinct proteins/peptides. Mass spectroscopy using a method described herein may detect 10-500 distinct proteins/peptides. Mass spectroscopy using a method described herein may detect 1-400 distinct proteins/peptides. Mass spectroscopy using a method described herein may detect 2-400 distinct proteins/peptides. Mass spectroscopy using a method described herein may detect 3-400 distinct proteins/peptides. Mass spectroscopy using a method described herein may detect 4-400 distinct proteins/peptides. Mass spectroscopy using a method described herein may detect 5-400 distinct proteins/peptides. Mass spectroscopy using a method described herein may detect 6-400 distinct proteins/peptides. Mass spectroscopy using a method described herein may detect 7-400 distinct proteins/peptides. Mass spectroscopy using a method described herein may detect 8-400 distinct proteins/peptides. Mass spectroscopy using a method described herein may detect 9-400 distinct proteins/peptides. Mass spectroscopy using a method described herein may detect 10-400 distinct proteins/peptides. Mass spectroscopy using a method described herein may detect 2-2000 distinct glycoproteins/glycopeptides. Mass spectroscopy using a method described herein may detect 3-1000 distinct glycoproteins/glycopeptides. Mass spectroscopy using a method described herein may detect 4-500 distinct glycoproteins/glycopeptides. Mass spectroscopy using a method described herein may detect 5-500 distinct glycoproteins/glycopeptides. Mass spectroscopy using a method described herein may detect 6-500 distinct glycoproteins/glycopeptides. Mass spectroscopy using a method described herein may detect 7-500 distinct glycoproteins/glycopeptides. Mass spectroscopy using a method described herein may detect 8-500 distinct glycoproteins/glycopeptides. Mass spectroscopy using a method described herein may detect 9-500 distinct glycoproteins/glycopeptides. Mass spectroscopy using a method described herein may detect 10-500 distinct glycoproteins/glycopeptides. Mass spectroscopy using a method described herein may detect 1-400 distinct glycoproteins/glycopeptides. Mass spectroscopy using a method described herein may detect 2-400 distinct glycoproteins/glycopeptides. Mass spectroscopy using a method described herein may detect 3-400 distinct glycoproteins/glycopeptides. Mass spectroscopy using a method described herein may detect 4-400 distinct glycoproteins/glycopeptides. Mass spectroscopy using a method described herein may detect 5-400 distinct glycoproteins/glycopeptides. Mass spectroscopy using a method described herein may detect 6-400 distinct glycoproteins/glycopeptides. Mass spectroscopy using a method described herein may detect 7-400 distinct glycoproteins/glycopeptides. Mass spectroscopy using a method described herein may detect 8-400 distinct glycoproteins/glycopeptides. Mass spectroscopy using a method described herein may detect 9-400 distinct glycoproteins/glycopeptides. Mass spectroscopy using a method described herein may detect 10-400 distinct glycoproteins/glycopeptides.

The proteomic data may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, 5000 or more distinct proteins or peptides. In some cases, the proteomic data may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, or 5000 distinct proteins or peptides.

The proteomic data may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, 5000 or more distinct glycoproteins or glycopeptides. In some cases, the proteomic data may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, or 5000 distinct glycoproteins or glycopeptides. Proteomic data may comprise 1-5000 distinct proteins/peptides. Proteomic data may comprise 2-2000 distinct proteins/peptides. Proteomic data may comprise 3-1000 distinct proteins/peptides. Proteomic data may comprise 4-500 distinct proteins/peptides. Proteomic data may comprise 5-500 distinct proteins/peptides. Proteomic data may comprise 6-500 distinct proteins/peptides. Proteomic data may comprise 7-500 distinct proteins/peptides. Proteomic data may comprise 8-500 distinct proteins/peptides. Proteomic data may comprise 9-500 distinct proteins/peptides. Proteomic data may comprise 10-500 distinct proteins/peptides. Proteomic data may comprise 1-400 distinct proteins/peptides. Proteomic data may comprise 2-400 distinct proteins/peptides. Proteomic data may comprise 3-400 distinct proteins/peptides. Proteomic data may comprise 4-400 distinct proteins/peptides. Proteomic data may comprise 5-400 distinct proteins/peptides. Proteomic data may comprise 6-400 distinct proteins/peptides. Proteomic data may comprise 7-400 distinct proteins/peptides. Proteomic data may comprise 8-400 distinct proteins/peptides. Proteomic data may comprise 9-400 distinct proteins/peptides. Proteomic data may comprise 10-400 distinct proteins/peptides. Proteomic data may comprise 2-2000 distinct glycoproteins/glycopeptides. Proteomic data may comprise 3-1000 distinct glycoproteins/glycopeptides. Proteomic data may comprise 4-500 distinct glycoproteins/glycopeptides. Proteomic data may comprise 5-500 distinct glycoproteins/glycopeptides. Proteomic data may comprise 6-500 distinct glycoproteins/glycopeptides. Proteomic data may comprise 7-500 distinct glycoproteins/glycopeptides. Proteomic data may comprise 8-500 distinct glycoproteins/glycopeptides. Proteomic data may comprise 9-500 distinct glycoproteins/glycopeptides. Proteomic data may comprise 10-500 distinct glycoproteins/glycopeptides. Proteomic data may comprise 1-400 distinct glycoproteins/glycopeptides. Proteomic data may comprise 2-400 distinct glycoproteins/glycopeptides. Proteomic data may comprise 3-400 distinct glycoproteins/glycopeptides. Proteomic data may comprise 4-400 distinct glycoproteins/glycopeptides. Proteomic data may comprise 5-400 distinct glycoproteins/glycopeptides. Proteomic data may comprise 6-400 distinct glycoproteins/glycopeptides. Proteomic data may comprise 7-400 distinct glycoproteins/glycopeptides. Proteomic data may comprise 8-400 distinct glycoproteins/glycopeptides. Proteomic data may comprise 9-400 distinct glycoproteins/glycopeptides. Proteomic data may comprise 10-400 distinct glycoproteins/glycopeptides.

In some instances, the data (the protein or proteomic data) may comprise a level of a biomolecule. The level of the biomolecule may be an absolute level of the biomolecule within the sample. The level of the biomolecule may be an absolute level of the biomolecule within a solution comprising the sample, biomolecule corona, or the biomolecules released from the biomolecule corona/particle. An absolute level of the biomolecule may be measured in mole; molarity (within the sample or the solution comprising the sample, biomolecule corona, or the biomolecules released from the biomolecule corona/particle); weight of the biomolecule divided by the volume of the sample or solution thereof; weight/mass of the biomolecule divided by the weight/mass of the sample; mass of the biomolecule; or any combinations thereof.

In some instances, the data may comprise a level of a reference biomolecule (of the internal standard described herein). The level of the reference biomolecule may be an absolute level of the reference biomolecule within a sample. The level of the reference biomolecule may be an absolute level of the reference biomolecule within a solution comprising the sample, biomolecule corona, or the reference biomolecules. An absolute level of the reference biomolecule may be measured in mole; molarity (within the sample or the solution comprising the sample, biomolecule corona, or the reference biomolecules); weight of the reference biomolecule divided by the volume of the sample or solution thereof; weight/mass of the reference biomolecule divided by the weight/mass of the sample; mass of the reference biomolecule; or any combinations thereof.

The data described herein may include glycan data. glycan data may include glycomic data. Glycomic data may involve data about glycan moieties. In some embodiments, the glycomic data is generated from a method described herein. In some embodiments, the glycomic data is analyzed by a method described herein.

Glycomic data may include information on the presence, absence, or amount of various glycan moieties. For example, glycomic data may include amounts of glycan moieties. A glycan moiety amount may be indicated as a concentration or quantity of glycan moieties, for example a concentration of a glycan moiety in a biofluid. A glycan moiety amount may be relative to another glycan moiety or to another biomolecule. Glycomic data may include information on the presence of glycan moieties. Glycomic data may include information on the absence of glycan moieties. Glycomic data may be distinguished by subtype, where each subtype includes a different type of glycan moieties.

Glycomic data generally includes data on a number of glycan moieties. For example, glycomic data may include information on the presence, absence, or amount of 1000 or more glycan moieties. In some cases, glycomic data may include information on the presence, absence, or amount of 5000, 10,000, 20,000, or more peptides, glycan moieties, or proteoforms. Glycomic data may even include up to about 1 million proteoforms. Glycomic data may include a range of glycan moieties, peptides, or proteoforms defined by any of the aforementioned numbers of glycan moieties, peptides, or proteoforms.

Glycomic data may be generated by any of a variety of methods. Generating glycomic data may include using a detection reagent that binds to a glycan moiety and yields a detectable signal. After use of a detection reagent that binds to a glycan moiety and yields a detectable signal, a readout may be obtained that is indicative of the presence, absence or amount of the glycan moiety. Generating glycomic data may include concentrating, filtering, or centrifuging a sample.

Glycomic data may be generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. Some examples of methods for generating glycomic data include using mass spectrometry, a glycan moiety chip, or a reverse-phased glycan moiety microarray. Glycomic data may also be generated using immunoassays such as enzyme-linked immunosorbent assays, western blots, dot blots, or immunohistochemistry. Generating glycomic data may involve use of an immunoassay panel.

In addition to any of the above methods, generating glycomic data may include contacting a sample with particles such that the particles adsorb biomolecules comprising proteins. The adsorbed proteins may be part of a biomolecule corona. Glycan moieties are then released from the adsorbed proteins and measured or identified in generating the glycomic data.

For generating the glycomic data, a solution comprising at least about 1 picogram (pg), 2 pg, 5 pg, 10 pg, 20 pg, 50 pg, 100 pg, 200 pg, 500 pg, 1 nanogram (ng), 2 ng, 5 ng, 10 ng, 20 ng, 50 ng, 100 ng, 200 ng, 500 ng, 1 microgram (μg), 2 μg, 5 μg, 10 μg, 20 μg, 50 μg, 100 μg, 200 μg, 500 μg, 1 milligram (mg), 2 mg, 5 mg, 10 mg, 20 mg, 50 mg, 100 mg, 200 mg, 500 mg, 1 gram or more of glycan moieties may be applied to the mass spectrometer. For generating the glycomic data, a solution comprising at least about 1 picogram (pg), 2 μg, 5 pg, 10 μg, 20 pg, 50 μg, 100 pg, 200 μg, 500 pg, 1 nanogram (ng), 2 ng, 5 ng, 10 ng, 20 ng, 50 ng, 100 ng, 200 ng, 500 ng, 1 microgram (μg), 2 μg, 5 μg, 10 μg, 20 μg, 50 μg, 100 μg, 200 μg, 500 μg, 1 milligram (mg), 2 mg, 5 mg, 10 mg, 20 mg, 50 mg, 100 mg, 200 mg, 500 mg, or 1 gram of glycan moieties may be applied to the mass spectrometer. For generating the glycomic data, a solution comprising at least about 1×10\hat{ }−20 moles, 1×10\hat{ }−19 moles, 1×10\hat{ }−18 moles, 1×10\hat{ }−17 moles, 1×10\hat{ }−16 moles, 1×10\hat{ }−15 moles, 1×10\hat{ }−14 moles, 1×10\hat{ }−13 moles, 1×10\hat{ }−12 moles, 1×10\hat{ }−11 moles, 1×10\hat{ }−10 moles, 1×10\hat{ }−9 moles, 1×10\hat{ }−8 moles, 1×10\hat{ }−7 moles, 1×10\hat{ }−6 moles, 1×10\hat{ }−5 moles, 1×10\hat{ }−4 moles, 1×10\hat{ }−3 moles, 1×10\hat{ }−2 moles, 1×10\hat{ }−1 moles, 1×10\hat{ }0 moles, 1×10\hat{ }1 moles, 1×10\hat{ }2 moles, 1×10\hat{ }3 moles, 1×10\hat{ }4 moles, 1×10\hat{ }5 moles, 1×10\hat{ }6 moles, 1×10\hat{ }7 moles, 1×10\hat{ }8 moles, 1×10\hat{ }9 moles, 1×10\hat{ }10 moles, 1×10\hat{ }11 moles, 1×10\hat{ }12 moles, 1×10\hat{ }13 moles, 1×10\hat{ }14 moles, 1×10\hat{ }15 moles, 1×10\hat{ }16 moles, 1×10\hat{ }17 moles, 1×10\hat{ }18 moles, 1×10\hat{ }19 moles, or 1×10\hat{ }20 moles of glycan moieties may be applied to the mass spectrometer. In some cases, For generating the glycomic data, a solution comprising at most about 1×10\hat{ }−20 moles, 1×10\hat{ }−19 moles, 1×10\hat{ }−18 moles, 1×10\hat{ }−17 moles, 1×10\hat{ }−16 moles, 1×10\hat{ }−15 moles, 1×10\hat{ }−14 moles, 1×10\hat{ }−13 moles, 1×10\hat{ }−12 moles, 1×10\hat{ }−11 moles, 1×10\hat{ }−10 moles, 1×10\hat{ }−9 moles, 1×10\hat{ }−8 moles, 1×10\hat{ }−7 moles, 1×10\hat{ }−6 moles, 1×10\hat{ }−5 moles, 1×10\hat{ }−4 moles, 1×10\hat{ }−3 moles, 1×10\hat{ }−2 moles, 1×10\hat{ }−1 moles, 1×10\hat{ }0 moles, 1×10\hat{ }1 moles, 1×10\hat{ }2 moles, 1×10\hat{ }3 moles, 1×10\hat{ }4 moles, 1×10\hat{ }5 moles, 1×10\hat{ }6 moles, 1×10\hat{ }7 moles, 1×10\hat{ }8 moles, 1×10\hat{ }9 moles, 1×10\hat{ }10 moles, 1×10\hat{ }11 moles, 1×10\hat{ }12 moles, 1×10\hat{ }13 moles, 1×10\hat{ }14 moles, 1×10\hat{ }15 moles, 1×10\hat{ }16 moles, 1×10\hat{ }17 moles, 1×10\hat{ }18 moles, 1×10\hat{ }19 moles, or 1×10\hat{ }20 moles of glycan moieties may be applied to the mass spectrometer.

The mass spectroscopy using a method described herein may detect at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 1500, or 2000, distinct glycan moieties. In some cases, the mass spectroscopy using a method described herein may detect at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 1500, or 2000, or 5000 distinct glycan moieties. Mass spectroscopy using a method described herein may detect 1-5000 distinct glycan moieties. Mass spectroscopy using a method described herein may detect 2-2000 distinct glycan moieties. Mass spectroscopy using a method described herein may detect 3-1000 distinct glycan moieties. Mass spectroscopy using a method described herein may detect 4-500 distinct glycan moieties. Mass spectroscopy using a method described herein may detect 5-500 distinct glycan moieties. Mass spectroscopy using a method described herein may detect 6-500 distinct glycan moieties. Mass spectroscopy using a method described herein may detect 7-500 distinct glycan moieties. Mass spectroscopy using a method described herein may detect 8-500 distinct glycan moieties. Mass spectroscopy using a method described herein may detect 9-500 distinct glycan moieties. Mass spectroscopy using a method described herein may detect 10-500 distinct glycan moieties. Mass spectroscopy using a method described herein may detect 1-400 distinct glycan moieties. Mass spectroscopy using a method described herein may detect 2-400 distinct glycan moieties. Mass spectroscopy using a method described herein may detect 3-400 distinct glycan moieties. Mass spectroscopy using a method described herein may detect 4-400 distinct glycan moieties. Mass spectroscopy using a method described herein may detect 5-400 distinct glycan moieties. Mass spectroscopy using a method described herein may detect 6-400 distinct glycan moieties. Mass spectroscopy using a method described herein may detect 7-400 distinct glycan moieties. Mass spectroscopy using a method described herein may detect 8-400 distinct glycan moieties. Mass spectroscopy using a method described herein may detect 9-400 distinct glycan moieties. Mass spectroscopy using a method described herein may detect 10-400 distinct glycan moieties. Mass spectroscopy using a method described herein may detect 2-2000 distinct glycan moieties. Mass spectroscopy using a method described herein may detect 3-1000 distinct glycan moieties. Mass spectroscopy using a method described herein may detect 4-500 distinct glycan moieties. Mass spectroscopy using a method described herein may detect 5-500 distinct glycan moieties. Mass spectroscopy using a method described herein may detect 6-500 distinct glycan moieties. Mass spectroscopy using a method described herein may detect 7-500 distinct glycan moieties. Mass spectroscopy using a method described herein may detect 8-500 distinct glycan moieties. Mass spectroscopy using a method described herein may detect 9-500 distinct glycan moieties. Mass spectroscopy using a method described herein may detect 10-500 distinct glycan moieties. Mass spectroscopy using a method described herein may detect 1-400 distinct glycan moieties. Mass spectroscopy using a method described herein may detect 2-400 distinct glycan moieties. Mass spectroscopy using a method described herein may detect 3-400 distinct glycan moieties. Mass spectroscopy using a method described herein may detect 4-400 distinct glycan moieties. Mass spectroscopy using a method described herein may detect 5-400 distinct glycan moieties. Mass spectroscopy using a method described herein may detect 6-400 distinct glycan moieties. Mass spectroscopy using a method described herein may detect 7-400 distinct glycan moieties. Mass spectroscopy using a method described herein may detect 8-400 distinct glycan moieties. Mass spectroscopy using a method described herein may detect 9-400 distinct glycan moieties. Mass spectroscopy using a method described herein may detect 10-400 distinct glycan moieties.

The glycomic data may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, 5000 or more distinct glycan moieties. In some cases, the glycomic data may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, or 5000 distinct glycan moieties. Glycomic data may comprise 1-5000 distinct glycan moieties. Glycomic data may comprise 2-2000 distinct glycan moieties. Glycomic data may comprise 3-1000 distinct glycan moieties. Glycomic data may comprise 4-500 distinct glycan moieties. Glycomic data may comprise 5-500 distinct glycan moieties. Glycomic data may comprise 6-500 distinct glycan moieties. Glycomic data may comprise 7-500 distinct glycan moieties. Glycomic data may comprise 8-500 distinct glycan moieties. Glycomic data may comprise 9-500 distinct glycan moieties. Glycomic data may comprise 10-500 distinct glycan moieties. Glycomic data may comprise 1-400 distinct glycan moieties. Glycomic data may comprise 2-400 distinct glycan moieties. Glycomic data may comprise 3-400 distinct glycan moieties. Glycomic data may comprise 4-400 distinct glycan moieties. Glycomic data may comprise 5-400 distinct glycan moieties. Glycomic data may comprise 6-400 distinct glycan moieties. Glycomic data may comprise 7-400 distinct glycan moieties. Glycomic data may comprise 8-400 distinct glycan moieties. Glycomic data may comprise 9-400 distinct glycan moieties. Glycomic data may comprise 10-400 distinct glycan moieties. Glycomic data may comprise 2-2000 distinct glycan moieties. Glycomic data may comprise 3-1000 distinct glycan moieties. Glycomic data may comprise 4-500 distinct glycan moieties. Glycomic data may comprise 5-500 distinct glycan moieties. Glycomic data may comprise 6-500 distinct glycan moieties. Glycomic data may comprise 7-500 distinct glycan moieties. Glycomic data may comprise 8-500 distinct glycan moieties. Glycomic data may comprise 9-500 distinct glycan moieties. Glycomic data may comprise 10-500 distinct glycan moieties. Glycomic data may comprise 1-400 distinct glycan moieties. Glycomic data may comprise 2-400 distinct glycan moieties. Glycomic data may comprise 3-400 distinct glycan moieties. Glycomic data may comprise 4-400 distinct glycan moieties. Glycomic data may comprise 5-400 distinct glycan moieties. Glycomic data may comprise 6-400 distinct glycan moieties. Glycomic data may comprise 7-400 distinct glycan moieties. Glycomic data may comprise 8-400 distinct glycan moieties. Glycomic data may comprise 9-400 distinct glycan moieties. Glycomic data may comprise 10-400 distinct glycan moieties.

In some instances, the data may comprise a level of a protein/peptide (or glycoprotein/glycopeptide). The level of the protein/peptide (or glycoprotein/glycopeptide) may be an absolute level of the protein/peptide (or glycoprotein/glycopeptide) within the sample. The level of the protein/peptide (or glycoprotein/glycopeptide) may be an absolute level of the protein/peptide (or glycoprotein/glycopeptide) within a solution comprising the sample, protein/peptide (or glycoprotein/glycopeptide) corona, or the protein/peptide (or glycoprotein/glycopeptide) released from the biomolecule corona/particle. An absolute level of the protein/peptide (or glycoprotein/glycopeptide) may be measured in mole; molarity (within the sample or the solution comprising the sample, biomolecule corona, or the protein/peptide (or glycoprotein/glycopeptide) released from the biomolecule corona/particle); weight of the protein/peptide (or glycoprotein/glycopeptide) divided by the volume of the sample or solution thereof; weight/mass of the protein/peptide (or glycoprotein/glycopeptide) divided by the weight/mass of the sample; mass of the protein/peptide (or glycoprotein/glycopeptide); or any combinations thereof.

In some instances, the data may comprise a level of a reference protein/peptide (or glycoprotein/glycopeptide) of the internal standard described herein). The level of the reference protein/peptide (or glycoprotein/glycopeptide) may be an absolute level of the reference protein/peptide (or glycoprotein/glycopeptide) within a sample. The level of the reference protein/peptide (or glycoprotein/glycopeptide) may be an absolute level of the reference protein/peptide (or glycoprotein/glycopeptide) within a solution comprising the sample, reference biomolecule corona, or the reference protein/peptide (or glycoprotein/glycopeptide) released from the biomolecule corona/particle. An absolute level of the reference protein/peptide (or glycoprotein/glycopeptide) may be measured in mole; molarity (within the sample or the solution comprising the sample, biomolecule corona, or the reference protein/peptide (or glycoprotein/glycopeptide) released from the biomolecule corona/particle); weight of the reference protein/peptide (or glycoprotein/glycopeptide) divided by the volume of the sample or solution thereof; weight/mass of the reference protein/peptide (or glycoprotein/glycopeptide) divided by the weight/mass of the sample; mass of the reference protein/peptide (or glycoprotein/glycopeptide); or any combinations thereof.

In some instances, the data (the glycan or glycomic data) may comprise a level of a glycan moiety. The level of the glycan moiety may be an absolute level of the glycan moiety within the sample. The level of the glycan moiety may be an absolute level of the glycan moiety within a solution comprising the sample, glycan moiety corona, or the glycan moiety released from the biomolecule corona/particle. An absolute level of the glycan moiety may be measured in mole; molarity (within the sample or the solution comprising the sample, biomolecule corona, or the glycan moiety released from the biomolecule corona/particle); weight of the glycan moiety divided by the volume of the sample or solution thereof; weight/mass of the glycan moiety divided by the weight/mass of the sample; mass of the glycan moiety; or any combinations thereof.

In some instances, the data may comprise a level of a reference glycan moiety (of the internal standard described herein). The level of the reference glycan moiety may be an absolute level of the reference glycan moiety within a sample. The level of the reference glycan moiety may be an absolute level of the reference glycan moiety within a solution comprising the sample, reference biomolecule corona, or the reference glycan moiety. An absolute level of the reference glycan moiety may be measured in mole; molarity (within the sample or the solution comprising the sample, glycan moiety corona, or the reference glycan moiety); weight of the reference glycan moiety divided by the volume of the sample or solution thereof; weight/mass of the reference glycan moiety divided by the weight/mass of the sample; mass of the reference glycan moiety; or any combinations thereof.

When releasing a glycan moiety from the glycoprotein/glycopeptide using the enzyme described herein, the enzymatic reaction may be conducted in heavy water, and the glycosylated site in which the glycan moiety is released by the enzyme can be labeled with an isotope of the heavy water. The method may then generate a labeled de-glycosylated glycoprotein/glycopeptide. In some cases, the data of the method may comprise a level of the glycan moiety, the de-glycosylated glycoprotein/glycopeptide, the non-glycosylated protein/peptide, or a combination thereof. In some cases, the level of de-glycosylated glycoprotein/glycopeptide and the non-glycosylated protein/peptide may be used to calculate a ratio of the glycosylation at a glycosylation site of the glycoprotein/glycopeptide.

In some instance, mass spectroscopy may analyze a solution comprising a protein/peptide (or glycoprotein or glycopeptide). In some instance, mass spectroscopy may analyze a solution comprising a glycan moiety. In some instance, mass spectroscopy may analyze a solution comprising both a protein/peptide (or glycoprotein or glycopeptide) and a glycan moiety. In some instance, mass spectroscopy may analyze a solution comprising a protein/peptide (or glycoprotein or glycopeptide) but not a glycan moiety (not attached to the protein/peptide and glycosylated thereof). In some instance, mass spectroscopy may analyze a solution comprising a glycan moiety but not a protein/peptide (or glycoprotein or glycopeptide). In some cases, a data set may comprise protein or proteomic data. In some cases, a data set may comprise glycomic data. In some cases, a data set may comprise protein/proteomic data and glycomic data. In some cases, a data set may comprise protein/proteomic data but not glycomic data. In some cases, a data set may comprise glycomic data but not protein/proteomic data.

f. Computer Systems

Certain aspects of the methods described herein may be carried out using a computer system. For example, proteomic or glycomic data analysis may be carried out using a computer system. Likewise, proteomic or glycomic data may be obtained through the use of a computer system. A readout indicative of the presence, absence or amount of a biomolecule (e.g., protein or peptide or glycan moiety) may be obtained at least in part using a computer system. The computer system may be used to carry out a method of using a classifier to assign a label corresponding to a presence, absence, or likelihood of a disease state to proteomic data, or to identify proteomic data as indicative or as not indicative of the disease state. The computer system may generate a report identifying a likelihood of the subject having a disease state. The computer system may transmit the report. For example, a diagnostic laboratory may transmit a report regarding the disease state identification to a medical practitioner. A computer system may receive a report.

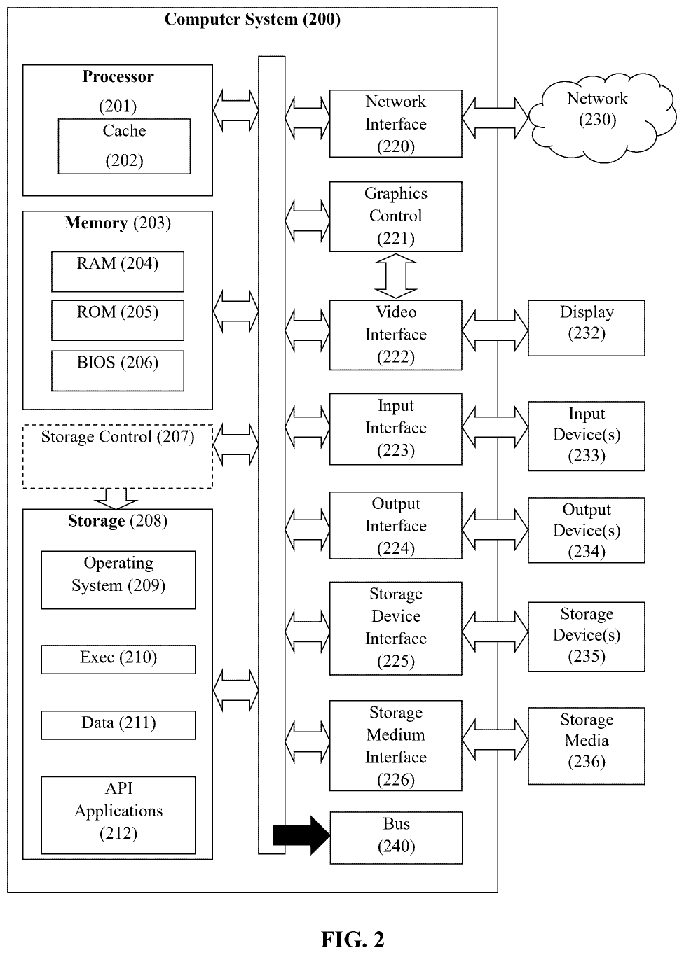

A computer system that carries out a method described herein may include some or all of the components shown in FIG. 2. Referring to FIG. 2, a block diagram is shown depicting an example of a machine that includes a computer system 200 (e.g., a processing or computing system) within which a set of instructions can execute for causing a device to perform or execute any one or more of the aspects and/or methodologies for static code scheduling of the present disclosure. The components in FIG. 2 are examples, and do not limit the scope of use or functionality of any hardware, software, embedded logic component, or a combination of two or more such components implementing particular aspects.

Computer system 200 may include one or more processors 201, a memory 203, and a storage 408 that communicate with each other, and with other components, via a bus 240. The bus 240 may also link a display 232, one or more input devices 233 (which may, for example, include a keypad, a keyboard, a mouse, a stylus, etc.), one or more output devices 234, one or more storage devices 235, and various tangible storage media 236. All of these elements may interface directly or via one or more interfaces or adaptors to the bus 240. For instance, the various tangible storage media 236 can interface with the bus 240 via storage medium interface 226. Computer system 200 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 200 includes one or more processor(s) 201 (e.g., central processing units (CPUs) or general-purpose graphics processing units (GPGPUs)) that carry out functions. Processor(s) 201 optionally contains a cache memory unit 202 for temporary local storage of instructions, data, or computer addresses. Processor(s) 201 are configured to assist in execution of computer readable instructions. Computer system 200 may provide functionality for the components depicted in FIG. 2 as a result of the processor(s) 201 executing non-transitory, processor-executable instructions embodied in one or more tangible computer-readable storage media, such as memory 203, storage 208, storage devices 235, and/or storage medium 236. The computer-readable media may store software that implements particular aspects, and processor(s) 201 may execute the software. Memory 203 may read the software from one or more other computer-readable media (such as mass storage device(s) 235, 236) or from one or more other sources through a suitable interface, such as network interface 220. The software may cause processor(s) 201 to carry out one or more processes or one or more steps of one or more processes described or illustrated herein. Carrying out such processes or steps may include defining data structures stored in memory 203 and modifying the data structures as directed by the software.

The memory 203 may include various components (e.g., machine readable media) including, but not limited to, a random access memory component (e.g., RAM 204) (e.g., static RAM (SRAM), dynamic RAM (DRAM), ferroelectric random access memory (FRAM), phase-change random access memory (PRAM), etc.), a read-only memory component (e.g., ROM 205), and any combinations thereof. ROM 205 may act to communicate data and instructions unidirectionally to processor(s) 201, and RAM 204 may act to communicate data and instructions bidirectionally with processor(s) 201. ROM 205 and RAM 204 may include any suitable tangible computer-readable media described below. In one example, a basic input/output system 206 (BIOS), including basic routines that help to transfer information between elements within computer system 200, such as during start-up, may be stored in the memory 203.

Fixed storage 208 is connected bidirectionally to processor(s) 201, optionally through storage control unit 207. Fixed storage 208 provides additional data storage capacity and may also include any suitable tangible computer-readable media described herein. Storage 208 may be used to store operating system 209, executable(s) 210, data 211, applications 212 (application programs), and the like. Storage 208 can also include an optical disk drive, a solid-state memory device (e.g., flash-based systems), or a combination of any of the above. Information in storage 208 may, in appropriate cases, be incorporated as virtual memory in memory 203.

In one example, storage device(s) 235 may be removably interfaced with computer system 400 (e.g., via an external port connector (not shown)) via a storage device interface 225. Particularly, storage device(s) 235 and an associated machine-readable medium may provide non-volatile and/or volatile storage of machine-readable instructions, data structures, program modules, and/or other data for the computer system 200. In one example, software may reside, completely or partially, within a machine-readable medium on storage device(s) 235. In another example, software may reside, completely or partially, within processor(s) 201.

Bus 240 connects a wide variety of subsystems. Herein, reference to a bus may encompass one or more digital signal lines serving a common function, where appropriate. Bus 240 may be any of several types of bus structures including, but not limited to, a memory bus, a memory controller, a peripheral bus, a local bus, and any combinations thereof, using any of a variety of bus architectures. As an example and not by way of limitation, such architectures may include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, or any combination thereof.

Computer system 200 may also include an input device 233. In one example, a user of computer system 200 may enter commands and/or other information into computer system 200 via input device(s) 233. Examples of an input device(s) 233 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), or any combinations thereof. The input device may include a Kinect, Leap Motion, or the like. Input device(s) 233 may be interfaced to bus 240 via any of a variety of input interfaces 223 (e.g., input interface 223) including, but not limited to, serial, parallel, game port, USB, FIREWIRE, THUNDERBOLT, or any combination of the above.

When computer system 200 is connected to network 230, computer system 200 may communicate with other devices, specifically mobile devices and enterprise systems, distributed computing systems, cloud storage systems, cloud computing systems, and the like, connected to network 230. Communications to and from computer system 200 may be sent through network interface 220. For example, network interface 220 may receive incoming communications (such as requests or responses from other devices) in the form of one or more packets (such as Internet Protocol (IP) packets) from network 230, and computer system 200 may store the incoming communications in memory 203 for processing. Computer system 200 may similarly store outgoing communications (such as requests or responses to other devices) in the form of one or more packets in memory 203 and communicated to network 230 from network interface 220. Processor(s) 201 may access these communication packets stored in memory 203 for processing.

Examples of the network interface 220 include, but are not limited to, a network interface card, a modem, or any combination thereof. Examples of a network 230 or network segment 230 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, or any combinations thereof. A network, such as network 230, may employ a wired and/or a wireless mode of communication. In general, any network topology may be used.

Information and data can be displayed through a display 232. Examples of a display 232 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, or any combinations thereof. The display 232 can interface to the processor(s) 201, memory 203, and fixed storage 208, as well as other devices, such as input device(s) 233, via the bus 240. The display 232 is linked to the bus 240 via a video interface 222, and transport of data between the display 232 and the bus 240 can be controlled via the graphics control 221. The display may be a video projector. The display may be a head-mounted display (HMID) such as a VR headset. Suitable VR headsets may include HTC Vive, Oculus Rift, Samsung Gear VR, Microsoft HoloLens, Razer OSVR, FOVE VR, Zeiss VR One, Avegant Glyph, Freefly VR headset, or the like. The display may include a combination of devices such as those disclosed herein.

In addition to a display 232, computer system 200 may include one or more other peripheral output devices 234 including, but not limited to, an audio speaker, a printer, a storage device, or any combinations thereof. Such peripheral output devices may be connected to the bus 240 via an output interface 224. Examples of an output interface 224 include, but are not limited to, a serial port, a parallel connection, a USB port, a FIREWIRE port, a THUNDERBOLT port, or any combinations thereof.

In addition or as an alternative, computer system 200 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with aspects disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with aspects disclosed herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with aspects disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium. An example storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers may include those with booklet, slate, or convertible configurations.

The computing device may include an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. The operating system may be provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some cases, the platforms, systems, media, or methods disclosed herein include one or more non-transitory computer readable storage media encoded with a program including instructions executable by an operating system of a computer system. The computer system may be networked. A computer readable storage medium may be a tangible component of a computing device. A computer readable storage medium may be removable from a computing device. A computer readable storage medium may include any of, by way of non-limiting examples, CD-ROMs, DVDs, flash memory devices, solid state memory, magnetic disk drives, magnetic tape drives, optical disk drives, distributed computing systems including cloud computing systems and services, or the like. In some cases, the program and instructions are permanently, substantially permanently, semi-permanently, or non-transitorily encoded on the media.

The methods disclosed herein may include the use of one or more classifiers. The classifier may be used to identify a subject as having a disease state based on data or measurements disclosed here. For example, glycoprotein data (e.g., measurements of glycoprotein groups or glycopeptides) may be used to identify a subject as having a disease state or as not having the disease state. The glycoprotein data may be obtained or analyzed, at least in part, using a software package such as MSFragger2, PEAKS, PEAKS PTM, or Byonic3.

g. Disease Identification

The methods described herein may be used to predict or identify a disease state. A disease may include a disorder. A disease state may include having a comorbidity related to a disease or disorder. A reference to whether a subject has a disease state or not may include the subject being healthy. A healthy state may exclude a disease state. For example, a healthy state may exclude having cancer. A disease state may exclude being healthy. A disease state may include a disease or disorder such as cancer.

A cancer may have a cancer stage. The cancer stage may be an early stage or a late stage. The cancer may comprise a stage 0 cancer, stage I cancer, a stage II cancer, a stage III cancer, a stage IV cancer. The early cancer stage may comprise a stage I cancer, a stage II cancer, or a stage III cancer. A late-stage cancer may comprise a stage IV cancer.

A subject may lack cancer but be at risk of developing the cancer. For example, the subject may have neoplastic cells that have the potential to develop into a cancer.

A stage I cancer may be localized to one area, tissue, or organ. A stage I cancer may not have grown deeply into a tissue adjacent its origin. A stage I cancer may not have grown into a lymph node. A stage I cancer may comprise stage I cancer with a tumor with at most about 2 centimeters (cm) in cross section. A stage I cancer may also comprise a cancer with a tumor with at least about 2 cm in cross section. A stage I cancer may comprise a cancer with a tumor with at most about 4 cm in cross section. A stage I cancer may comprise a cancer with a tumor with about 2 to 4 cm in cross section.

A stage II or III cancer may comprise a cancer that has grown into a tissue adjacent its origin or lymph node. In some instances, a stage II cancer may comprise a cancer that has not grown into a lymph node. A stage II cancer is larger in size, volume, or weight than a stage I cancer. A stage II cancer may comprise a cancer with a tumor with at least about 4 cm in cross section and has not spread to a lymph node. A stage II cancer may comprise a cancer that has spread to at most about 3 lymph nodes. A stage II cancer may comprise a cancer with at most about 2 cm in cross section and has spread to at most about 3 lymph nodes. A stage II cancer may comprise a cancer from about 2 to 4 in cross section and has spread to at most about 3 lymph nodes. A stage II cancer may comprise a cancer with at least about 4 in cross section and has spread to at most about 3 lymph nodes.

A stage III cancer may be larger in size, volume, or weight than a stage II cancer. A stage III cancer may have a deeper penetration into a tissue than a stage II cancer does. In some instances, a stage III cancer may have spread to at least 4 lymph nodes. A stage III cancer may comprise a cancer that is at most about 2 cm in cross section and has spread to at least about 4 lymph nodes. A stage III cancer may comprise a cancer that is about 2 to 4 cm in cross section and has spread to at least about 4 lymph nodes. A stage III cancer may comprise a cancer that is at least about 4 cm in cross section and has spread to at least about 4 lymph nodes.

In some instances, a stage IV cancer may comprise a cancer that has spread to other organs or parts of a subject, relative to the part/tissue the cancer originates (e.g., a metastatic cancer). In some cases, a stage IV cancer may comprise an advanced or metastatic cancer.

In some cases, the method described may determine whether a subject has a cancer, the stage of the cancer, or the risk of developing the cancer. The method described may also determine whether a sample is associated with the cancer, the stage of the cancer, or the risk of having the cancer.

Examples of cancer include lung cancer, colon cancer, pancreatic cancer, liver cancer, ovarian cancer, breast cancer, prostate cancer, melanoma, bladder cancer, lymphoma, leukemia, renal cancer, or uterine cancer. An example of lung cancer is non-small cell lung cancer (NSCLC).

In some cases, a lung cancer type may comprise small cell lung cancer (SCLC), non-small cell lung cancer (NSCLC), lung carcinoid tumors, adenoid cystic carcinomas, lymphomas, or sarcomas. In some instances, a method may predict the disease outcome of a subject with two of SCLC, NSCLC, lung carcinoid tumors, adenoid cystic carcinomas, lymphomas, and sarcomas. In some instances, a NSCLC may comprise adenocarcinoma of the lung, squamous carcinoma of the lung, large cell (undifferentiated) carcinoma, adenosquamous carcinoma, sarcomatoid carcinoma, or any combinations thereof.

A protein/peptide (or glycosylated thereof) may be associated with the cancer described herein. Using the methods described herein, comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000 or more distinct proteins/peptides (or glycosylated thereof) may be identified to be associated with the cancer. Using the methods described herein, comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or 1000 distinct proteins/peptide s(or glycosylated thereof) may be identified to be associated with the cancer.

The protein/peptide (or glycosylated thereof) associated with the cancer may comprise ALB, CASP3, CD44, CDH1, CYCS, ENO2, EXT2, FBN1, FH, FN1, GNAQ, GSTP1, HABP2, HSP90AA1, HSPB1, IDH2, IGF1, IGF2, IGFBP3, ITGB1, KRAS, MAPK1, MINPP1, MMP1, MMP14, MMP2, MT-CO2, MXRA5, PEPN12, PHB, PLAD2A, PRKAR1A, PRKCA, PTPRJ, SDHA, SERPIANA3, SLC2A1, SLC9A9, SLMAP, SOD2, SPP1, SRC, STAT3, TGFB1, THBS1, THOA, TIMP1, TYMP, VEGFC, or a combination thereof. The protein/peptide (or glycosylated thereof) associated with the cancer may comprise ALB. The protein/peptide (or glycosylated thereof) associated with the cancer may comprise CASP3. The protein/peptide (or glycosylated thereof) associated with the cancer may comprise CD44. The protein/peptide (or glycosylated thereof) associated with the cancer may comprise CDH1. The protein/peptide (or glycosylated thereof) associated with the cancer may comprise CYCS. The protein/peptide (or glycosylated thereof) associated with the cancer may comprise ENO2. The protein/peptide (or glycosylated thereof) associated with the cancer may comprise EXT2. The protein/peptide (or glycosylated thereof) associated with the cancer may comprise FBN1. The protein/peptide (or glycosylated thereof) associated with the cancer may comprise FH. The protein/peptide (or glycosylated thereof) associated with the cancer may comprise FN1. The protein/peptide (or glycosylated thereof) associated with the cancer may comprise GNAQ. The protein/peptide (or glycosylated thereof) associated with the cancer may comprise GSTP1. The protein/peptide (or glycosylated thereof) associated with the cancer may comprise HABP2. The protein/peptide (or glycosylated thereof) associated with the cancer may comprise HSP90AA1. The protein/peptide (or glycosylated thereof) associated with the cancer may comprise HSPB1. The protein/peptide (or glycosylated thereof) associated with the cancer may comprise IDH2. The protein/peptide (or glycosylated thereof) associated with the cancer may comprise IGF1. The protein/peptide (or glycosylated thereof) associated with the cancer may comprise IGF2. The protein/peptide (or glycosylated thereof) associated with the cancer may comprise IGFBP3. The protein/peptide (or glycosylated thereof) associated with the cancer may comprise ITGB1. The protein/peptide (or glycosylated thereof) associated with the cancer may comprise KRAS. The protein/peptide (or glycosylated thereof) associated with the cancer may comprise MAPK1. The protein/peptide (or glycosylated thereof) associated with the cancer may comprise MINPP1. The protein/peptide (or glycosylated thereof) associated with the cancer may comprise MMP1. The protein/peptide (or glycosylated thereof) associated with the cancer may comprise MMP14. The protein/peptide (or glycosylated thereof) associated with the cancer may comprise MMP2. The protein/peptide (or glycosylated thereof) associated with the cancer may comprise MT-CO2. The protein/peptide (or glycosylated thereof) associated with the cancer may comprise MXRA5. The protein/peptide (or glycosylated thereof) associated with the cancer may comprise PEPN12. The protein/peptide (or glycosylated thereof) associated with the cancer may comprise PHB. The protein/peptide (or glycosylated thereof) associated with the cancer may comprise PLAD2 Å. The protein/peptide (or glycosylated thereof) associated with the cancer may comprise PRKAR1 Å. The protein/peptide (or glycosylated thereof) associated with the cancer may comprise PRKCA. The protein/peptide (or glycosylated thereof) associated with the cancer may comprise PTPRJ. The protein/peptide (or glycosylated thereof) associated with the cancer may comprise SDHA. The protein/peptide (or glycosylated thereof) associated with the cancer may comprise SERPIANA3. The protein/peptide (or glycosylated thereof) associated with the cancer may comprise SLC2 Å1. The protein/peptide (or glycosylated thereof) associated with the cancer may comprise SLC9 Å9. The protein/peptide (or glycosylated thereof) associated with the cancer may comprise SLMAP. The protein/peptide (or glycosylated thereof) associated with the cancer may comprise SOD2. The protein/peptide (or glycosylated thereof) associated with the cancer may comprise SPP1. The protein/peptide (or glycosylated thereof) associated with the cancer may comprise SRC. The protein/peptide (or glycosylated thereof) associated with the cancer may comprise STAT3. The protein/peptide (or glycosylated thereof) associated with the cancer may comprise TGFB1. The protein/peptide (or glycosylated thereof) associated with the cancer may comprise THBS1. The protein/peptide (or glycosylated thereof) associated with the cancer may comprise THOA. The protein/peptide (or glycosylated thereof) associated with the cancer may comprise TIMP1. The protein/peptide (or glycosylated thereof) associated with the cancer may comprise TYMP. The protein/peptide (or glycosylated thereof) associated with the cancer may comprise VEGFC.

The method of determining a set of samples or a subject associated with the disease or disorder and/or disease state may comprise the analysis of the biomarkers (e.g., a biomolecule corona or any protein/peptides associated therewith or glycan moieties) of the at least one or two samples. A model may be trained with the one or more biomarkers using a classifier. The classifier may classify a sample is associated with a disease condition described herein or a risk thereof or a subject in which the sample is obtained from having the disease condition described herein or the risk thereof. For example, if a sample is determined to be associated with a disease condition or the risk thereof, the subject in which the sample is obtained is determined to have the disease condition or the risk thereof. The classifier may comprise supervised and unsupervised data analysis, machine learning, deep learning, dimension-reductio analysis, and/or clustering approaches. The classifier may comprise clustering approaches. The classifier may comprise deep learning. The classifier may comprise dimension-reduction analysis. The classifier may comprise machine learning. The classifier may comprise supervised data analysis. The classifier may comprise unsupervised data analysis. A classifier may comprise decision trees, hidden Markov analysis, hierarchical cluster analysis (HCA), K-means clustering, k-nearest neighbors, linear regression, logistic regression, naive bayes analysis, Partial least squares Discriminant Analysis (PLS-DA), polynomial regression, principal component analysis (PCA), random forest classification analysis, support vector machine (SVM), SVM for regression, or a combination thereof. A classifier may comprise decision trees. A classifier may comprise hidden Markov analysis. A classifier may comprise hierarchical cluster analysis (HCA). A classifier may comprise K-means clustering. A classifier may comprise k-nearest neighbors. A classifier may comprise linear regression. A classifier may comprise logistic regression. A classifier may comprise naive bayes analysis. A classifier may comprise Partial least squares Discriminant Analysis (PLS-DA). A classifier may comprise polynomial regression. A classifier may comprise principal component analysis (PCA). A classifier may comprise random forest classification analysis. A classifier may comprise support vector machine (SVM). A classifier may comprise SVM for regression.

In some cases, the proteins/peptides/glycan moieties (e.g., associated with the biomolecule corona) of each sample are compared/analyzed with each other to determine with statistical significance what patterns are common between the proteins of the subject to determine a set of proteins that is associated with the disease or disorder or disease state. In some cases, the proteins/peptide (e.g., associated with the biomolecule corona) of each sample are compared/analyzed with a dataset that is either having the same disease condition or a risk thereof. Any of such methods may be used to generate a classifier for use herein.

The classifier may be generated by removing or filtering out biomolecules associated with acute phase response. In some aspects, said classifier is configured to remove acute-phase-response bias or stress biomolecule bias. In some aspects, said classifier comprises features that relate to proteins or peptides (or the glycosylated versions thereof or the glycan moieties released from thereof). Said features may be selected to exclude acute-phase response and/or stress biomolecule bias in the sample. The classifier may comprise features (e.g., biomarker information) to distinguish between a disease condition or the risk thereof or other state (e.g., a healthy or comorbid state).

A model may be generated using any of the classifier and data described herein to determine if a sample or subject in which the sample is obtained from has a disease condition described herein pr a risk thereof. A model may be generated supervised and unsupervised data analysis, machine learning, deep learning, dimension-reductio analysis, and/or clustering approaches. A model may be generated by decision trees, hidden Markov analysis, hierarchical cluster analysis (HCA), K-means clustering, k-nearest neighbors, linear regression, logistic regression, naive bayes analysis, Partial least squares Discriminant Analysis (PLS-DA), polynomial regression, principal component analysis (PCA), random forest classification analysis, support vector machine (SVM), SVM for regression, or a combination thereof. A model may be trained with the one or more biomarkers using may comprise decision trees. A model may be trained with the one or more biomarkers using may comprise hidden Markov analysis. A model may be trained with the one or more biomarkers using may comprise hierarchical cluster analysis (HCA). A model may be trained with the one or more biomarkers using may comprise K-means clustering. A model may be trained with the one or more biomarkers using may comprise k-nearest neighbors. A model may be trained with the one or more biomarkers using may comprise linear regression. A model may be trained with the one or more biomarkers using may comprise logistic regression. A model may be trained with the one or more biomarkers using may comprise naive bayes analysis. A model may be trained with the one or more biomarkers using may comprise Partial least squares Discriminant Analysis (PLS-DA). A model may be trained with the one or more biomarkers using may comprise polynomial regression. A model may be trained with the one or more biomarkers using may comprise principal component analysis (PCA). A model may be trained with the one or more biomarkers using may comprise random forest classification analysis. A model may be trained with the one or more biomarkers using may comprise support vector machine (SVM). A model may be trained with the one or more biomarkers using may comprise SVM for regression. A method described herein may include use of the model. A method may include generating the model.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include Generative models; Low-density separation; Graph-based methods; and Co-training. Reinforcement learning concepts may include Temporal difference learning; Q-learning; Learning Automata; and SARSA. Deep learning concepts may include Deep belief networks; Deep Boltzmann machines; Deep Convolutional neural networks; Deep Recurrent neural networks; and Hierarchical temporal memory.

The methods described herein may include use of a classifier to identify or distinguish a disease condition or the risk thereof such as cancer (e.g., lung cancer or NSCLC). The classifier may distinguish the disease condition or the risk thereof from a comorbidity such as a chronic lung disorder, chronic obstructive pulmonary disease, emphysema, cardiovascular disease, hypertension, pulmonary fibrosis, or asthma.

In some instances, the method described herein may comprise determining whether a level of a biomolecule is different from a threshold level, wherein a difference of the level of a biomolecule and the threshold level indicates that the sample is associated with a disease condition described herein or a risk thereof or a subject in which the sample is obtained from having the disease condition described herein or the risk thereof. The level of a biomolecule or the threshold may comprise any level measurement described herein. For example, the level of a biomolecule or threshold level may be any absolute level measurement described herein.

The threshold level may comprise a level of a reference biomolecule of the internal standard described herein. For example, the threshold level may be the level of the reference biomolecule of the internal standard of the data described (e.g., the protein or proteomic data). When comparing a difference of a biomolecule (such as a modified biomolecule) with the level of the reference biomolecule, the reference biomolecule may be the unmodified form of the biomolecule. For example, a post-translationally modified protein or peptide within the sample or a solution comprising thereof may be compared to a reference molecule that is a unmodified form of the protein or peptide. In some cases, a glycoprotein or glycopeptide within the sample or a solution comprising thereof may be compared to a reference molecule that is a unmodified form of the protein or peptide. In some cases, a glycoprotein or glycopeptide within the sample or a solution comprising thereof may be compared to a reference molecule that is a non-glycosylated form of the protein or peptide. In other cases, the biomolecule and the reference biomolecule may have the same modification. For example, the biomolecule may be a glycoprotein/glycopeptide, and the reference biomolecule may also be a glycoprotein/glycopeptide.

The protein/peptide/glycan moiety and the reference versions thereof may have at least about at least about 60%, at least about 65%, at least about 70%, at least about 75%, at least about 80%, at least about 85% at least about 90% at least about 95%, at least about 96%, at least about 97%, at least about 98%, at least about 99%, or 100% sequence identity with each other. In some cases, the protein/peptide/glycan moiety and the reference versions thereof may have at most about at least about 60%, at least about 65%, at least about 70%, at least about 75% at least about 80%, at least about 85% at least about 90% at least about 95%, at least about 96% at least about 97% at least about 98% at least about 99% or 100% sequence identity with each other.

The threshold level may be determined when the internal standard (e.g., a glycoprotein or glycopeptide) is added into the sample or a solution comprising the sample, the biomolecule corona, the biomolecules or particle by the methods described herein.

In some cases, a difference of the level of the biomolecule and the internal standard (e.g., the threshold level) that is at least about 1%, 2%, 3%, 4%, 5%, 6%, 7%, 8%, 9%, 10%, 20%, 300%, 40%, 50%, 60%, 70%, 80%, 90%, 1-fold, 2-fold, 3-fold, 4-fold, 5-fold, 6-fold, 7-fold, 8-fold, 9-fold, 10-fold, 100-fold, 1000-fold, 10000-fold or more may indicate that a sample is associated with a disease condition described herein or a risk thereof or a subject in which the sample is obtained from having the disease condition described herein or the risk thereof. In some cases, a difference of the level of the biomolecule and the internal standard (e.g., the threshold level) that is at most about 1%, 2%, 3%, 4%, 5%, 6%, 7%, 8%, 9%, 10%, 20%, 300%, 40%, 50%, 60%, 70%, 80%, 90%, 1-fold, 2-fold, 3-fold, 4-fold, 5-fold, 6-fold, 7-fold, 8-fold, 9-fold, 10-fold, 100-fold, 1000-fold, or 10000-fold may indicate that a sample is associated with a disease condition described herein or a risk thereof or a subject in which the sample is obtained from having the disease condition described herein or the risk thereof.

In some cases, the threshold level may be pre-determined. For example, a pre-determined threshold level may be generated by determining the level of a biomolecule within a sample derived from a subject that is healthy or without a disease condition or the risk thereof; or a solution comprising the sample, the biomolecule corona, or biomolecules/particle derived from the subject that is healthy or without a disease condition or the risk thereof. When determining if a subject is associated with the disease or the risk thereof, the level of the biomolecule present within a sample derived from the subject (or a solution comprising the sample, the biomolecule corona, or biomolecules/particle derived from the subject) is determined and compared with the pre-determined threshold level; and a difference between the level of the biomolecule and the threshold level described herein can indicate that the subject has the disease or disease condition.

In some instances, the method described herein may comprise determining whether a level of a biomolecule is the same or substantially the same as a threshold level, wherein the same or substantially the same levels of the biomolecule and the threshold level indicates that the sample is associated with a disease condition described herein or a risk thereof or a subject in which the sample is obtained from having the disease condition described herein or the risk thereof. In such case, a pre-determined threshold level may be generated by determining the level of a biomolecule within a sample derived from a subject that has a disease condition or the risk thereof; or a solution comprising the sample, biomolecule corona, or biomolecules/particle derived from the subject that has the disease condition or the risk thereof. When determining if a different subject is associated with the disease or the risk thereof, the level of the biomolecule present within a sample derived from the different subject (or a solution comprising the sample, the biomolecule corona, or biomolecules/particle derived from the subject) is determined and compared with the pre-determined threshold level; and a difference that is at most about 1%, 2%, 3%, 4%, 5%, 6%, 7%, 8%, 9%, 10%, 20%, or 30% between the level of the biomolecule and the threshold level (the level of the biomolecule and the threshold level are the same or substantially the same) can indicate that the subject has the disease or disease condition.

In some instances, the method described herein may comprise determining whether a level of glycosylation of a glycoprotein or glycopeptide is different from a threshold glycosylation level, wherein a difference of the glycosylation level the glycoprotein or glycopeptide and the threshold level indicates that the sample is associated with a disease condition described herein or a risk thereof or a subject in which the sample is obtained from having the disease condition described herein or the risk thereof. The glycosylation level of the glycoprotein or glycopeptide or the threshold level may comprise a ratio of the glycosylation at a glycosylation site of the glycoprotein/glycopeptide calculated by the methods described herein.

For example, the threshold glycosylation level (the ratio of the glycosylation at the glycosylation site of the glycoprotein/glycopeptide) may be generated using a sample or a solution comprising the sample or a biomolecule derived from the sample obtained from a subject that is healthy or without a disease condition or risk thereof; and the threshold glycosylation level is compared to the glycosylation level of a second subject, wherein if the difference between the threshold glycosylation level and the glycosylation level of the second subject is at least about 1%, 2%, 3%, 4%, 5%, 6%, 7%, 8%, 9%, 10%, 20%, 30%, 40%, 50%, 60%, 70%, 80%, 90%, 1-fold, 2-fold, 3-fold, 4-fold, 5-fold, 6-fold, 7-fold, 8-fold, 9-fold, 10-fold, 100-fold, 1000-fold, 10000-fold or more, the second subject is determined to have the disease condition described herein or the risk thereof. In some cases, wherein the if difference between the threshold glycosylation level and the glycosylation level of the second subject is at most about 1%, 2%, 3%, 4%, 5%, 6%, 7%, 8%, 9%, 10%, 20%, 30%, 40%, 50%, 60%, 70%, 80%, 90%, 1-fold, 2-fold, 3-fold, 4-fold, 5-fold, 6-fold, 7-fold, 8-fold, 9-fold, 10-fold, 100-fold, 1000-fold, or 10000-fold, the second subject is determined to have the disease condition described herein or the risk thereof.

In some cases, the threshold glycosylation level (the ratio of the glycosylation at the glycosylation site of the glycoprotein/glycopeptide) may be generated using a sample or a solution comprising the sample or a biomolecule derived from the sample obtained from a subject that is has a disease condition or risk thereof; and the threshold glycosylation level is compared to the glycosylation level of a second subject, wherein if the difference between the threshold glycosylation level and the glycosylation level of the second subject is at least at most about 1%, 2%, 3%, 4%, 5%, 6%, 7%, 8%, 9%, 10%, 20%, or 30%, the second subject is determined to have the disease condition described herein or the risk thereof.

In some cases, a biomarker may be used to determine whether a subject has a disease condition or risk thereof. In some cases, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 500, 1000, 5000, 10000 or more biomarkers may be used to determine whether a subject has a disease condition or risk thereof. In some cases, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 500, 1000, 5000, or 10000 biomarkers may be used to determine whether a subject has a disease condition or risk thereof.

In some instances, the method may have a sensitivity for classifying a sample is associated with a disease condition described herein or a risk thereof or a subject in which the sample is obtained from having the disease condition described herein or the risk thereof. In some cases, the sensitivity of the method may be the true positive rate of the method for classifying a sample or the subject in which the sample is obtained from as being positive for having the disease condition or the risk thereof (or negative for vice versa). In some cases, the sensitivity of the method may be calculated by:
Sensitivity may=Number of samples (or the subject in which the sample is obtained from) with the disease condition (or the risk thereof) and identified to be positive for the disease condition (or the risk thereof)/(Number of samples with the disease condition and identified to be positive for the disease condition (or the risk thereof)+Number of samples with the disease condition but identified to be negative for the disease condition (or the risk thereof)); or
Sensitivity may=Number of samples (or the subject in which the sample is obtained from) with the disease condition (or the risk thereof) and identified to be positive for the disease condition (or the risk thereof)/Total number of samples (or the subject in which the sample is obtained from) with the disease condition (or the risk thereof)

In some cases, the sensitivity of a method for classifying a sample as being positive or negative for a disease condition (or the risk thereof) may be at least about 50%. In some cases, the sensitivity of the method may be at least about 51%. In some cases, the sensitivity of the method may be at least about 52%. In some cases, the sensitivity of the method may be at least about 53%. In some cases, the sensitivity of the method may be at least about 54%. In some cases, the sensitivity of the method may be at least about 55%. In some cases, the sensitivity of the method may be at least about 56%. In some cases, the sensitivity of the method may be at least about 57%. In some cases, the sensitivity of the method may be at least about 58%. In some cases, the sensitivity of the method may be at least about 59%. In some cases, the sensitivity of the method may be at least about 60%. In some cases, the sensitivity of the method may be at least about 61%. In some cases, the sensitivity of the method may be at least about 62%. In some cases, the sensitivity of the method may be at least about 63%. In some cases, the sensitivity of the method may be at least about 64%. In some cases, the sensitivity of the method may be at least about 65%. In some cases, the sensitivity of the method may be at least about 66%. In some cases, the sensitivity of the method may be at least about 67%. In some cases, the sensitivity of the method may be at least about 68%. In some cases, the sensitivity of the method may be at least about 69%. In some cases, the sensitivity of the method may be at least about 70%. In some cases, the sensitivity of the method may be at least about 71%. In some cases, the sensitivity of the method may be at least about 72%. In some cases, the sensitivity of the method may be at least about 73%. In some cases, the sensitivity of the method may be at least about 74%. In some cases, the sensitivity of the method may be at least about 75%. In some cases, the sensitivity of the method may be at least about 76%. In some cases, the sensitivity of the method may be at least about 77%. In some cases, the sensitivity of the method may be at least about 78%. In some cases, the sensitivity of the method may be at least about 79%. In some cases, the sensitivity of the method may be at least about 80%. In some cases, the sensitivity of the method may be at least about 81%. In some cases, the sensitivity of the method may be at least about 82%. In some cases, the sensitivity of the method may be at least about 83%. In some cases, the sensitivity of the method may be at least about 84%. In some cases, the sensitivity of the method may be at least about 85%. In some cases, the sensitivity of the method may be at least about 86%. In some cases, the sensitivity of the method may be at least about 87%. In some cases, the sensitivity of the method may be at least about 88%. In some cases, the sensitivity of the method may be at least about 89%. In some cases, the sensitivity of the method may be at least about 90%. In some cases, the sensitivity of the method may be at least about 91%. In some cases, the sensitivity of the method may be at least about 92%. In some cases, the sensitivity of the method may be at least about 93%. In some cases, the sensitivity of the method may be at least about 94%. In some cases, the sensitivity of the method may be at least about 95%. In some cases, the sensitivity of the method may be at least about 96%. In some cases, the sensitivity of the method may be at least about 97%. In some cases, the sensitivity of the method may be at least about 98%. In some cases, the sensitivity of the method may be at least about 99%.

In some instances, the method may have a specificity for classifying a sample as being positive or negative for a disease condition (or the risk thereof). In some cases, the specificity of the method may be the true negative rate of the method for classifying a sample as being positive or negative for a disease condition (or the risk thereof). In some cases, the specificity of the method may be calculated by:
Specificity=Number of samples (or the subject in which the sample is obtained from) without the disease condition (or the risk thereof) and identified to be negative for the disease condition (or the risk thereof)/(Number of samples (or the subject in which the sample is obtained from) without the disease condition (or the risk thereof) and identified to be negative for the disease condition (or the risk thereof)+Number of samples (or the subject in which the sample is obtained from) without the disease condition (or the risk thereof) but identified to be positive for the disease condition (or the risk thereof)); or
Specificity=Number of samples (or the subject in which the sample is obtained from) without the disease condition (or the risk thereof) and identified to be negative for the disease condition (or the risk thereof)/Total number of samples (or the subject in which the sample is obtained from) without the disease condition (or the risk thereof).

In some cases, the specificity of a method for classifying a sample as being positive or negative for a disease condition (or the risk thereof) may be at least about 50%. In some cases, the specificity of the method may be at least about 51%. In some cases, the specificity of the method may be at least about 52%. In some cases, the specificity of the method may be at least about 53%. In some cases, the specificity of the method may be at least about 54%. In some cases, the specificity of the method may be at least about 55%. In some cases, the specificity of the method may be at least about 56%. In some cases, the specificity of the method may be at least about 57%. In some cases, the specificity of the method may be at least about 58%. In some cases, the specificity of the method may be at least about 59%. In some cases, the specificity of the method may be at least about 60%. In some cases, the specificity of the method may be at least about 61%. In some cases, the specificity of the method may be at least about 62%. In some cases, the specificity of the method may be at least about 63%. In some cases, the specificity of the method may be at least about 64%. In some cases, the specificity of the method may be at least about 65%. In some cases, the specificity of the method may be at least about 66%. In some cases, the specificity of the method may be at least about 67%. In some cases, the specificity of the method may be at least about 68%. In some cases, the specificity of the method may be at least about 69%. In some cases, the specificity of the method may be at least about 70%. In some cases, the specificity of the method may be at least about 71%. In some cases, the specificity of the method may be at least about 72%. In some cases, the specificity of the method may be at least about 73%. In some cases, the specificity of the method may be at least about 74%. In some cases, the specificity of the method may be at least about 75%. In some cases, the specificity of the method may be at least about 76%. In some cases, the specificity of the method may be at least about 77%. In some cases, the specificity of the method may be at least about 78%. In some cases, the specificity of the method may be at least about 79%. In some cases, the specificity of the method may be at least about 80%. In some cases, the specificity of the method may be at least about 81%. In some cases, the specificity of the method may be at least about 82%. In some cases, the specificity of the method may be at least about 83%. In some cases, the specificity of the method may be at least about 84%. In some cases, the specificity of the method may be at least about 85%. In some cases, the specificity of the method may be at least about 86%. In some cases, the specificity of the method may be at least about 87%. In some cases, the specificity of the method may be at least about 88%. In some cases, the specificity of the method may be at least about 89%. In some cases, the specificity of the method may be at least about 90%. In some cases, the specificity of the method may be at least about 91%. In some cases, the specificity of the method may be at least about 92%. In some cases, the specificity of the method may be at least about 93%. In some cases, the specificity of the method may be at least about 94%. In some cases, the specificity of the method may be at least about 95%. In some cases, the specificity of the method may be at least about 96%. In some cases, the specificity of the method may be at least about 97%. In some cases, the specificity of the method may be at least about 98%. In some cases, the specificity of the method may be at least about 99%.

In some cases, a method may have any specificity and sensitivity thereof for classifying a sample as being positive or negative for a disease condition (or the risk thereof).

i. Subjects and Treatment

The methods described herein may be used to identify a subject as likely to have a disease state or not. The subject may be a vertebrate. The subject may be a mammal. The subject may be a primate. The subject may be a human. The subject may be male or female. The subject may have a disease state. For example, the subject may have a disease or disorder, a comorbidity of a disease or disorder, or may be healthy.

A sample may be obtained from the subject for purposes of identifying a disease state in the subject. The subject may be suspected of having the disease state or as not having the disease state. The method may be used to confirm or refute the suspected disease state.

In some cases, the subject is monitored. For example, information about a likelihood of the subject having a disease state may be used to determine to monitor a subject without providing a treatment to the subject. In other circumstances, the subject may be monitored while receiving treatment to see if a disease state in the subject improves.

When the subject is identified as not having the disease state, the subject may avoid an otherwise unfavorable disease treatment (and associated side effects of the disease treatment) or is able to avoid having to be biopsied or tested invasively for the disease state. When the subject is identified as not having the disease state, the subject may be monitored without receiving a treatment. When the subject is identified as not having the disease state, the subject may be monitored without receiving a biopsy. In some cases, the subject identified as not having the disease state may be treated with palliative care such as a pharmaceutical composition for pain. In some cases, the subject is identified as having another disease different from the initially suspected disease state and is provided treatment for the other disease.

When the subject is identified as having the disease state, the subject may be provided a treatment for the disease state. For example, if the disease state is cancer, the subject may be provided a cancer treatment. Examples of treatments may include surgery, organ transplantation, administration of a pharmaceutical composition, radiation therapy, chemotherapy, immunotherapy, hormone therapy, monoclonal antibody treatment, stem cell transplantation, gene therapy, or chimeric antigen receptor (CAR)-T cell or transgenic T cell administration. Any of these treatments may be to remove cancer.

When the subject is identified as having the disease state, the subject may be further evaluated for the disease state. For example, a subject suspected of having the disease state may be subjected to a biopsy after a method disclosed herein indicates that he or she may have the disease state.

Some cases include recommending a treatment or monitoring of the subject. For example, a medical practitioner may receive a report generated by a method described herein. The report may indicate a likelihood of the subject having a disease state. The medical practitioner may then provide or recommend the treatment or monitoring to the subject or to another medical practitioner. Some cases include recommending a treatment for the subject. Some cases include recommending monitoring of the subject.

An example of a disease that may be tested includes cancer. The cancer may be a lung cancer such as non-small cell-lung cancer. The cancer may be stage 1. The cancer may be stage 2. The cancer may be stage 1 or 2 (e.g., early stage). The cancer may be stage 3. The cancer may be stage 4. The cancer may be any of stages 1-4. The cancer may be an unidentified stage. For example, a subject may undergo a blood test when the subject is suspected of having a cancer such as lung cancer. The subject may have not yet received a computed tomography (CT) scan to check for lung nodules, may be under consideration for treatment with an immune checkpoint inhibitor (ICI), or may have potentially resectable cancer.

### EXAMPLES

The following illustrative examples are representative of aspects of classifiers, systems, or methods described herein and are not meant to be limiting in any way.

### Example 1. GlycoIQ Platform Development

A bioinformatics approach was employed to develop a method that utilized an existing Proteograph™ dataset on lung nodule subjects with 3 software packages (e.g., MSFragger, PEAKS, and Protein Metrics). This approached initially determined the level of detected glycosylated proteins and peptides in Proteograph™ datasets and the quantitative differences between native and glycosylated versions of the same proteins. This approach also verified observed glycoproteins with published studies on lung cancer. Proteograph™ refers to a method or system that captures or enriches biomolecules using nanoparticles.

An initial in-silico experiment was conducted. This experiment analyzed the 2021 lung feasibility study data on 212 subjects with multiple glycol search engines to locate glycosylated proteins and the glycan. This experiment determined whether any detected glycosylated proteins in the lung feasibility dataset were also present within the initial lung nodule proteomics dataset. There is opportunity to maximize 1^{st} mover advantage in generating Proteograph™ data including glycoproteins at scale. There is also opportunity to enhance platform with N-linked and O-linked glycosylation enrichment to expand biomarker discovery space.

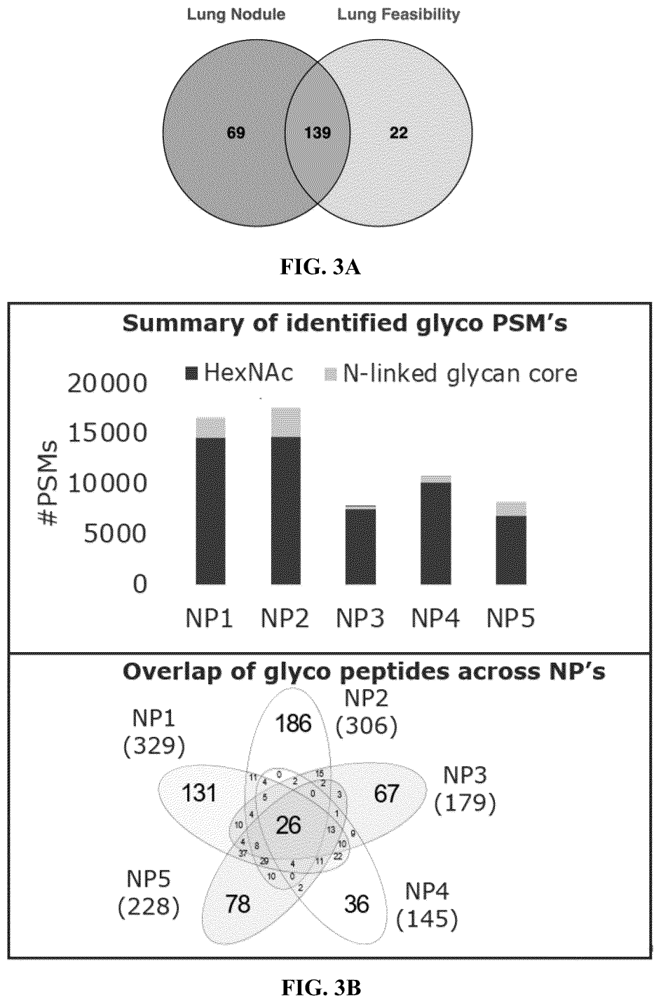

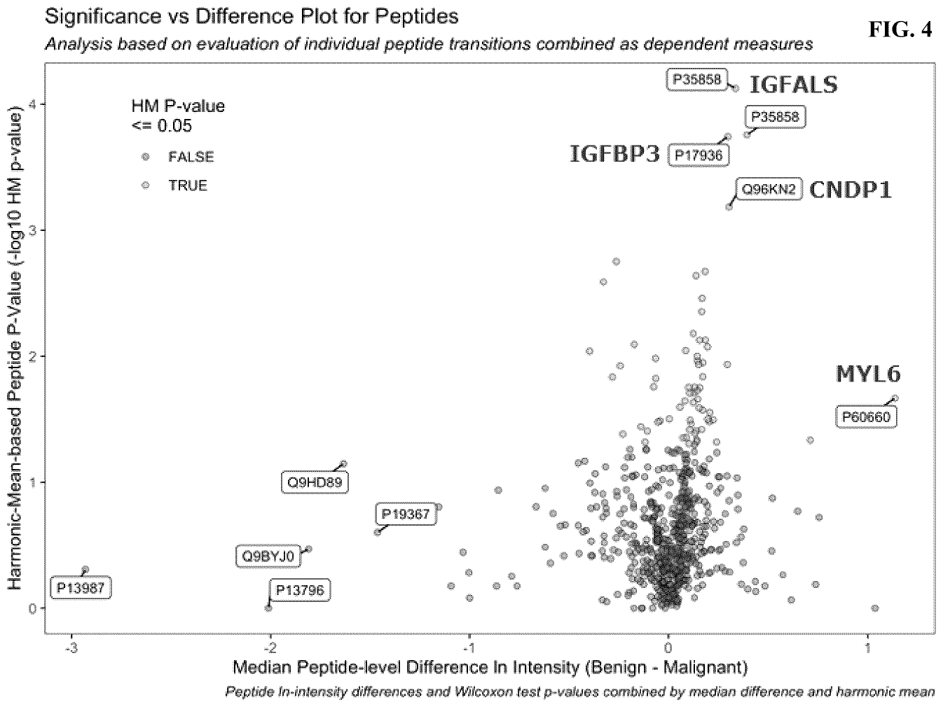

The Proteograph™ derived proteomics data was found to be rich with glycoproteins. FIG. 3A illustrates a bioinformatic approach for examining the glycoprotein data set. Existing Proteograph™ dataset on lung nodule subjects were analyzed with software: MSFragger, PEAKS, or Protein Metrics. The dataset was analyzed for level of glycosylated proteins or glycosylated peptides detected in Proteograph™ datasets; quantitative differences between native or glycosylated versions of same proteins or peptides; and verification of observed glycoproteins with published studies. Samples obtained from 212 subjects were analyzed with multiple glyco search engines to locate glycosylated proteins and the glycan. Glycosylated proteins in Lung Feasibility dataset and Lung Nodule dataset were identified. 139 glycosylated proteins were detected in both datasets. FIG. 3B illustrates a summary of identified glyco Peptide Spectral Matches (“PSMs”). The total and specific glycoprotein enrichment was found to be differentiated across nanoparticle chemistry. Thousands of PSMs for hundreds of detected glyco-peptides indicated a highly abundant signal for the detected peptides. High redundancy resulted from the stochastic data collection of the most abundant peptides first. A comparison was made of the uniquely identified glycosylated peptides on each NP across 212 subjects. FIG. 3B shows the overlap of glyco peptides across NPs. NP1 and NP5 detected the largest number of unique glycosylated peptides.

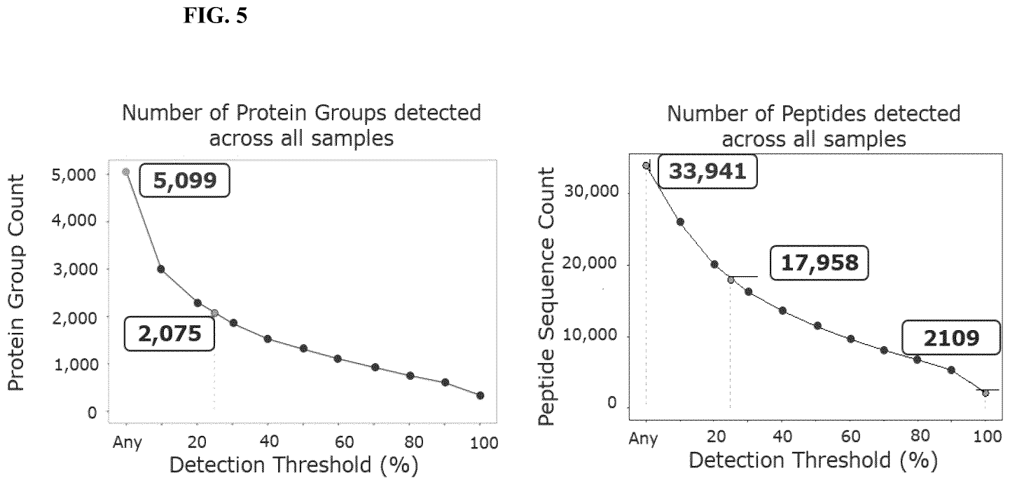

Early analysis highlighted the potential value of proteoform detection. FIG. 4 shows the significance and difference plot for peptides. The most statistically significant protein in the lung discovery study data was also detected as a glyco-peptide in the lung feasibility data set. The glycopeptide was detected for IGFALS with a glycosylation site at amino acid 368. This glycopeptide was previously reported in the proteomics literature. Ongoing analysis may determine the robustness of IGFALS detection and relevance to disease.

### Example 2. Incorporation of Glycoproteome Detection into Large Scale Unbiased Proteomics Studies Utilizing Nanoparticles

Challenges in early cancer detection have led to poor survival rates, highlighting the need for early diagnostic test development. Biomarkers measured in liquid biopsies offer a less invasive and accessible strategy for early cancer detection. Analyte degradation and dilution in complex biological matrix limit high specificity and sensitivity measurements, making biomarker discovery from blood a formidable challenge. A proteomics platform has been developed.

Deep unbiased proteomics analysis in the platform is facilitated by recent advances in sample preparation (i.e., Seer's Proteograph™ Product Suite) coupled with improved mass spectrometry instrument sensitivity and speed. Together, they provide the ability to quantify thousands of proteins from human plasma without compromising throughput or reproducibility. Additionally, Seer's Proteograph™ nanoparticle technology specifically captures unique proteoforms in the corona formation and allows for comprehensive assessment of the circulating glycoproteome.

### Methods

In a recent study to detect cancer related biomarkers, 212 subject K2EDTA plasma samples (116 cancer subjects and 96 healthy control subjects) were analyzed, prospectively collected following an IRB approved protocol, on the Seer Proteograph™ platform using a five-nanoparticle panel commercially available from Seer. Resulting peptides were analyzed on a Bruker timsTOF Pro mass spectrometer in data dependent (DDA-PASEF) mode coupled with a Dionex Utilimate 3000 LC generating a 60 min gradient on a 50 cm uPAC pillar array column (Pharmafluidics).

Data was searched with MaxQuant¹ utilizing the following search parameters: 0.1% peptide/protein FDR search, default timsTOF parameters searched against complete UniProt SwissProt human proteome database with 50% reversed decoys and contaminants. All datafiles for each nanoparticle were searched together. Due to the large number of total datafiles (1,200) comprising all subjects and all nanoparticles, only datafiles for an individual nanoparticle across all 212 subjects were searched due to computational limitations.

The dataset of 212 subjects was also searched for the presence of glycosylated proteins utilizing MSFragger², PEAKS PTM implemented in PEAKS online (Bioinformatics Solutions) and Byonic³ (Protein Metrics) using default N-glycosylation parameters on each platform. All searches were performed against the Human UniProt database with 50% reversed decoys and contaminants. All 1,200 datafiles were searched with MSFragger utilizing the published open search parameters² for N-glycosylation and filtered at 1% FDR (peptide/protein). Complete data from nanoparticles 1 and 2 (480 datafiles) were also searched with PEAKS online and filtered at 1% FDR (PSM) and −log 10p score>50.

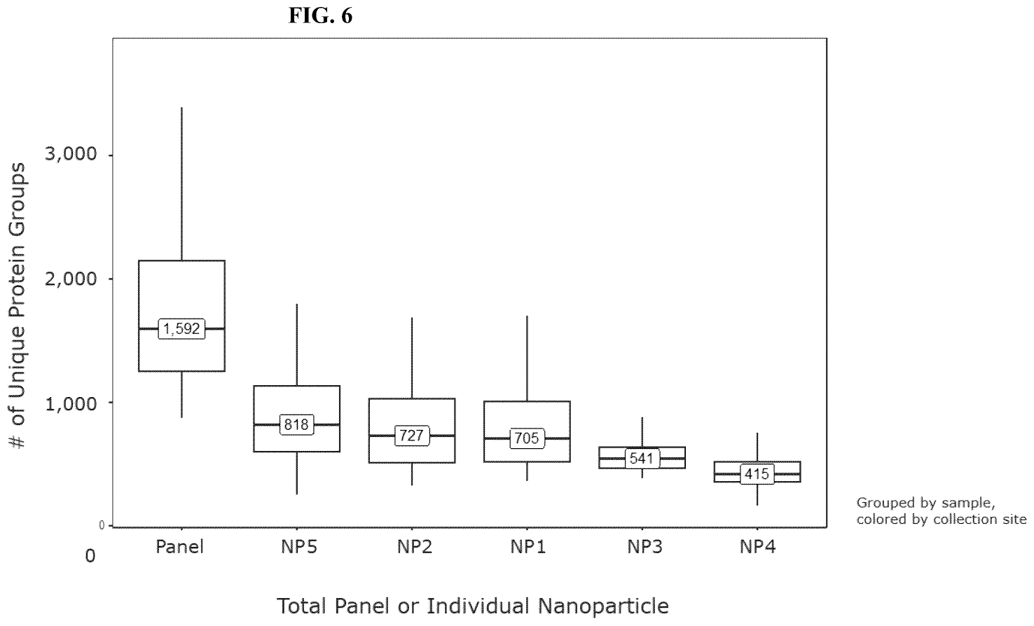

FIG. 5 shows 5,099 proteins groups and 33,941 peptides detected across all 5 nanoparticles for the 212 subject samples, with a median of 4 peptides per protein for proteins present in >25% of the samples utilizing depletion and fractionation but were generated in significantly less time per sample.

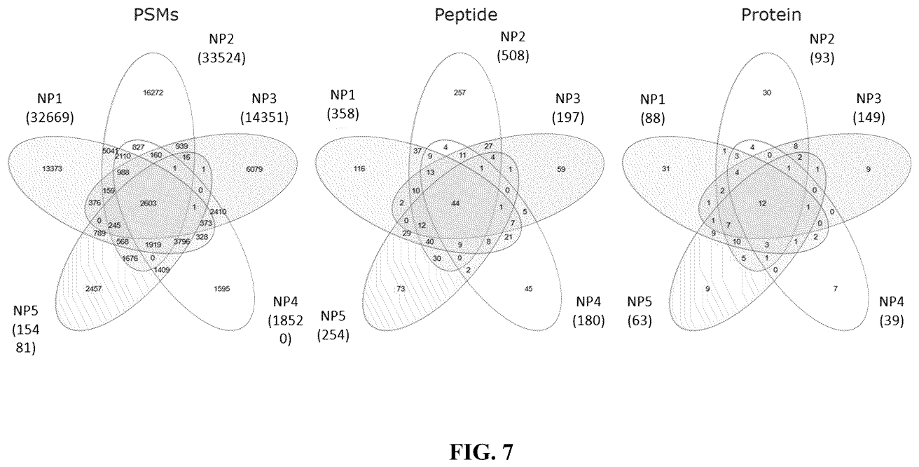

A median of 1,592 protein groups were detected across all 5 nanoparticles for all 212 subjects in this study (FIG. 6). NP5 provided the largest number, and most diverse, protein groups detected in any of the nanoparticles. Samples were grouped with connecting lines and colored by collections site. A high sample overall or for a given particle is generally then high in the other nanoparticles as well.

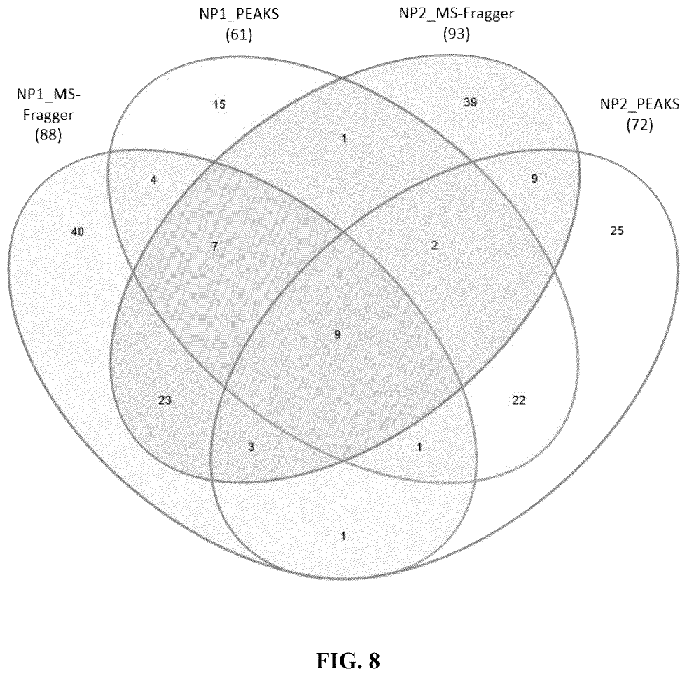

Individual nanoparticles yielded both complementary and common glycoprotein identifications. (a) Peptide-spectral matches (PSMs) corresponded to (b) glycopeptides and (c) glycoproteins inferred from MSFragger searches across the five NP panel from ˜1200 datafiles (FIG. 7). A total of 66,511 glyco-PSMs from 877 unique peptides derived from 165 proteins were identified at 1% FDR, with NP1 and NP2 datasets accounting for ˜80% of observed unique N-linked glycopeptides.

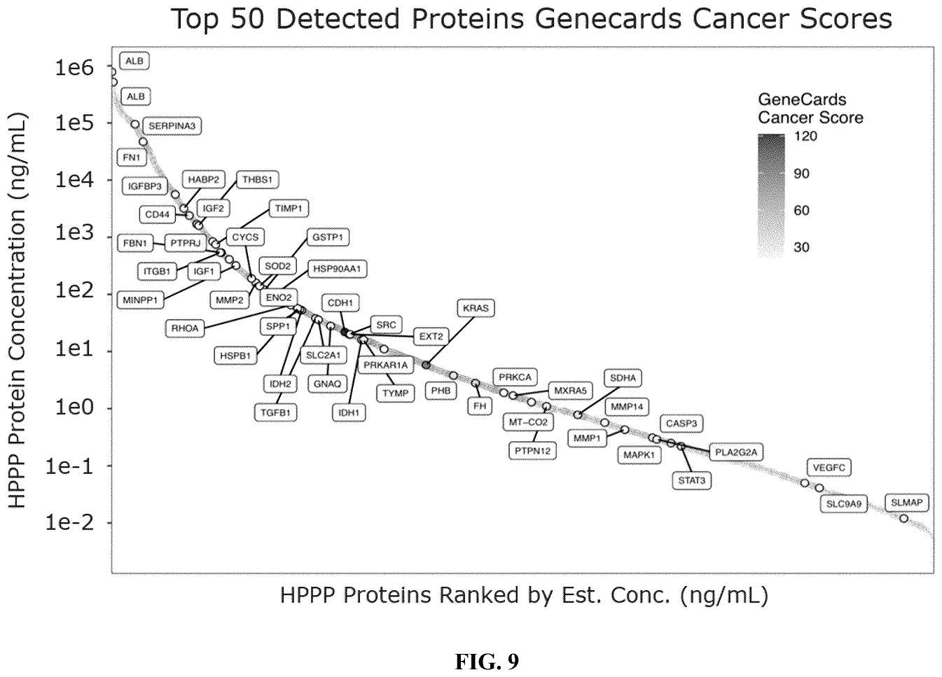

FIG. 8 shows a Venn diagram of proteins from which glycopeptides were detected in PEAKS or MSFragger glyco searches across nanoparticles NP1 and NP2 (480 files). Around 75% (66/88) of proteins from which glycopeptides were identified via MSFragger and ˜46% (28/61) of proteins with glycopeptides identified via PEAKS were also measured in the Max Quant label free quant (LFQ) search. Little overlap was observed between two algorithms at a glycopeptide level—Three common peptides in NP1 and one in NP2. This may have resulted from high disparity in the repertoire of glycoforms searched by the two algorithms.

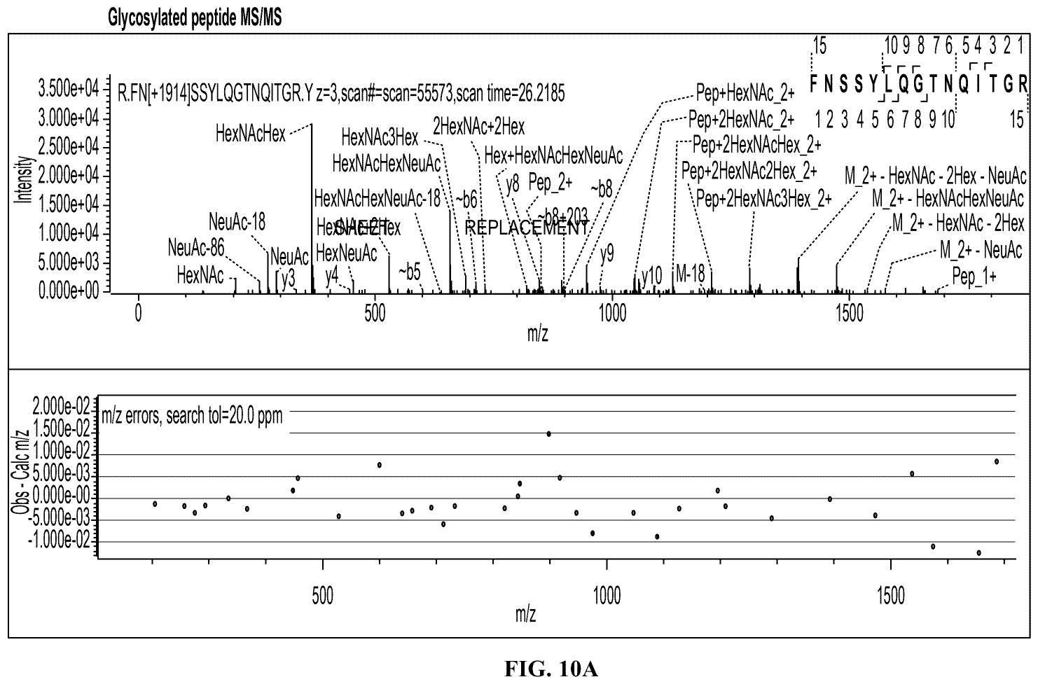

The detected 5,099 protein groups were mapped to the HPPP database, which illustrated the wide range of reported protein concentrations (8 orders of magnitude) measured with the Proteograph™ nanoparticle technology and timsTOF Pro instrumentation. Additionally, a Genecards⁶ analysis was performed to determine the cancer associated proteins detected. FIG. 9 highlights the top 50 proteins, of which ˜40% have known plasma concentrations of <10 ng/mL.

Glycopeptides were found in PEAKS searches from 40 of the cancer associated proteins detected in the study. 42 cancer associated proteins detected in the study also had glycopeptides detected by MSFragger with agreement between the two algorithms on 16 overlapping glycoproteins. Of these 66 unique proteins for which glycopeptides were detected across both algorithms, 53 had multiple previously reported or predicted glycosites (UniProt).

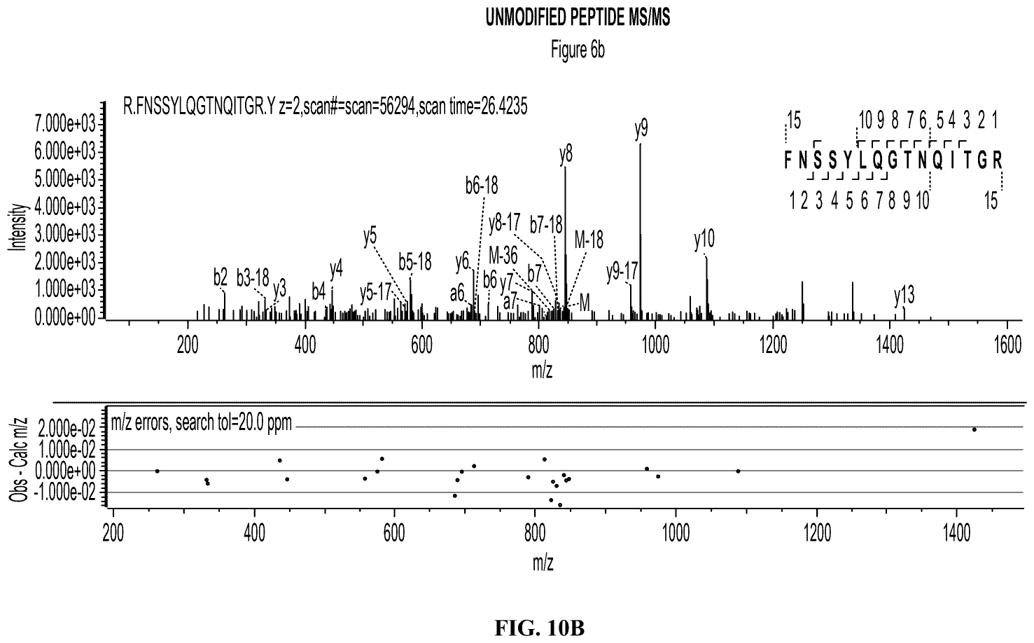

MS/MS spectrum from a (a) glycopeptide—FN(HexNAc-4Hex-5NeuAc-1)SSYLQGTNQITGR and (b) its unmodified counterpart, derived from Apolipoprotein-B (APOB), also shortlisted in the Genecard “cancer” search (FIGS. 10A-10B). The spectrum visualized from Byonic (Protein Metrics) following an N-glycan search of a single NP1 file. The APOB glycosite captured by this peptide, N1523 has been reported in targeted glycoproteomics experiments deploying enrichment strategies. Glyco-peptide ions along with N-acetylhexosamine (HexNAc) and N-acetylneuraminic acid (NeuAc) ions were detected in the MS/MS spectrum, consistent with the expected glycopeptide fragments expected to result from collision induced dissociation (CID).

### CONCLUSIONS

Unbiased proteomics of a large cohort of subjects utilizing Proteograph™ and timsTOF technologies identified >5,000 proteins and hundreds of glycosylated proteins in a single experiment. The simultaneous detection of native and glycosylated versions of the same proteins improved the analysis of differential expression of protein versions to detect associations with disease progression. Many of the glycosylated peptides detected were derived from glycoproteins and may have an association with cancer. Glyco-searches with newly developed bioinformatics tools may be computationally expensive but do scale to >1,000 files. Each search engine detected complementary glyco-peptides/proteins that improved the overall detection rate and resulted in high confident identifications for the consensus identifications.

### Example 3. Combination of the Unique Functions from timsTOF and ZenoTOF Enables In-Depth Analysis of the Glycoproteome

### Introduction

Glycoproteomics aims to study protein glycosylation at site specific level to reveal the functions of this important PTM. Apart from the unprecedented sensitivity and sampling rates, timsTOF and ZenoTOF each provides specific features such as ion mobility separation and electron activated dissociation (EAD) enabling novel opportunities for glycoproteomics. However, the potential of these new instruments in glycoproteomics has not yet fully explored. In this example, glycopeptides were enriched for method development to achieve highest glycopeptide ID in timsTOF Pro2 and ZenoTOF 7600. Efforts were then specially focused on utilizing the ion mobility module in timsTOF and the EAD function in ZenoTOF to gain additional glyco-related information to increase the depth of analysis.

### Methods

Pooled plasma (BioIVT) was digested by iST sample digestion kit from PreOmics. Glycopeptides were enriched using HILICON iSPE HILIC SPE cartridges. 10 mg plasma equivalent of enriched glycopeptide was used for each injection. timsTOF Pro2 coupled with EvoSep and ZenoTOF 7600 coupled with Water M class LC system were used for data acquisition. LC-MS methods were optimized for the highest unique glycopeptide ID numbers and compared with results from default proteomic methods from both instruments. Data was analyzed by Byonic using the 117 N-glycans databases. The search results of glycopeptide ID were filtered through a Byonic Score cutoff of 250. Further analysis was conducted using in-house written R scripts.

### Preliminary Results

MS2 selection criteria and collision energy settings were tested to optimize the glycoproteomics methods in timsTOF Pro2 and ZenoTOF 7600 for the highest unique glycopeptide identification. An average of 737 unique glycopeptides and an average of 720 unique glycopeptides were identified from timsTOF Pro2 and ZenoTOF 7600 respectively. Compared to an average of 87 unique glycopeptides when using the Bruker's default proteomics setting, this is a more than 8-fold increase. When combined the results from three runs, both timsTOF and ZenoTOF identified more than 1,000 unique N-glycopeptides using the optimized methods.

To further investigate the utility of the EAD function on the ZenoTOF for glycoproteomic. CID and EAD fragmentation were combined, and about 10% to 15% more unique N-glycopeptides were obtained in addition to the results from CID only methods, suggesting that combining complimentary fragmentation methods in a single injection could boost glycopeptide identification. To further investigate the power of ion mobility on timsTOF Pro2 to resolve glycopeptide structural isomers, depending on the ionization conditions, in-source fragmentation of glycan moiety from the intact glycopeptides were detected and separated by ion mobility in the timsTOF. For example, two peaks of m/z at 657.234 were detected in the mobilogram, likely to be the a2-3 and a2-6 sialylated LacNAc structures. Sialidase treatment could be used to confirm such isomeric separation of glycan fragments in the mobilogram. Higher order of glycan fragments such as sialyl Lewis structures (m/z at 803.29) were also observed and showed a more complicated mobilogram.

The observation suggests additional glycomics information could be extracted from the glycoproteomics workflow. Glycoproteomics methods were optimized for unique glycopeptide identification. Instrument specific features were explored to extract additional dimension of glyco-information.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1.-64. (canceled)

65. A method, comprising:
(a) enriching a biofluid sample from a subject for glycoproteins or glycopeptides, wherein the glycoproteins or glycopeptides comprise glycan moieties;
(b) separating the glycoproteins or glycopeptides from proteins or peptides of the biofluid sample in conditions sufficient for isotopic labeling, thereby producing a mixture comprising labeled glycan moieties, labeled glycoproteins or glycopeptides, and unlabeled glycoproteins or glycopeptides;
(c) analyzing, using a mass spectrometry assay, the mixture to produce a dataset; and
(d) applying a trained machine learning classifier to the dataset to identify the biofluid sample as indicative of cancer or as not indicative of cancer.

66. The method of claim 65, wherein the separating the glycoproteins or glycopeptides from the proteins or peptides comprises using liquid chromatography, solid phase extraction (SPE), or gel electrophoresis.

67. The method of claim 66, wherein the liquid chromatography comprises hydrophilic interaction liquid chromatography (HILIC), electrostatic repulsion liquid chromatography (ERLIC) enrichments, high performance liquid chromatography (HPLC), supercritical fluid chromatography (SFC), Reverse phase liquid chromatography (RP-LC), or a combination thereof.

68. The method of claim 67, wherein the liquid chromatography comprises at least two of the HILIC, the ERLIC, the HPLC, the SFC, or the RP-LC.

69. The method of claim 66, wherein the gel electrophoresis comprises two-dimensional electrophoresis.

70. The method of claim 65, further comprising contacting the biofluid sample with particles to form biomolecule coronas comprising the glycoproteins or glycopeptides adsorbed to the particles.

71. The method of claim 70, further comprising separating the glycoproteins or glycopeptides adsorbed to the biomolecule coronas from the particles.

72. The method of claim 70, wherein the particles comprise at least 2 different types of particles.

73. The method of claim 70, wherein the particles comprise physiochemically distinct sets of particles.

74. The method of claim 73, wherein the physiochemically distinct particles of the physiochemically distinct sets of particles comprise lipid particles, metal particles, silica particles, or polymer particles.

75. The method of claim 73, wherein the physiochemically distinct particles comprise carboxylate particles, poly acrylic acid particles, dextran particles, polystyrene particles, dimethylamine particles, amino particles, silica particles, or N-(3-trimethoxysilylpropyl)diethylenetriamine particles.

76. The method of claim 65, wherein the conditions sufficient for isotopic labeling in (b) comprises heavy water that comprises an isotope, glycosylation enzymes, monosaccharaides that comprise an isotope, or a combination thereof.

77. The method of claim 76, wherein the heavy water comprises ¹⁸O.

78. The method of claim 65, further comprising calculating a ratio of an amount of the labeled glycoproteins or glycopeptides and an amount of the unlabeled glycoproteins or glycopeptides.

79. The method of claim 65, wherein the trained machine learning classifier comprises a supervised data analysis, an unsupervised data analysis, a machine learning, a deep learning, a dimension reduction analysis, a clustering analysis, or a combination thereof.

80. The method of claim 79, wherein the clustering analysis comprises a hierarchical cluster analysis, a principal component analysis, a partial least squares discriminant analysis, a random forest classification analysis, a support vector machine analysis, a k-nearest neighbors analysis, a naive bayes analysis, a K-means clustering analysis, a hidden Markov analysis, or a combination thereof.

81. The method of claim 65, wherein the mass spectrometry assay comprises tandem mass spectrometry or liquid chromatography-mass spectrometry (LCMS).

82. The method of claim 65, wherein the cancer comprises non-small cell lung cancer (NSCLC).

83. The method of claim 82, wherein the NSCLC comprises stage 1 NSCLC, stage 2 NSCLC, stage 3 NSCLC, or stage 4 NSCLC.

84. The method of claim 65, wherein the trained machine learning classifier comprises features to distinguish between early-stage NSCLC and late-stage NSCLC.

85. A system, comprising:
one or more processors; and
one or more memories storing machine-executable code that, when executed, cause the one or more processors to:
(a) enrich a biofluid sample from a subject for glycoproteins or glycopeptides, wherein the glycoproteins or glycopeptides comprise glycan moieties;
(b) separate the glycoproteins or glycopeptides from proteins or peptides of the biofluid sample in conditions sufficient for isotopic labeling, thereby producing a mixture comprising labeled glycan moieties, labeled glycoproteins or glycopeptides, and unlabeled glycoproteins or glycopeptides;
(c) analyze, using a mass spectrometry assay, the mixture to produce a dataset; and
(d) apply a trained machine learning classifier to the dataset to identify the biofluid sample as indicative of cancer or as not indicative of cancer.
